(19) United States
(12) Patent Application Publication
(10) Pub. No.: US 2017/0305986 A1
(43) Pub. Date: Oct. 26, 2017

(54) NOVEL TGF-ALPHA MUTANT PROTEINS

(71) Applicant: NEUROREPAIR, INC., Encinitas, CA (US)

(72) Inventors: Matthew KLIPSTEIN, Encinitas, CA (US); Hwai Wen CHANG, San Marcos, CA (US); Gerhard FREY, San Diego, CA (US); Jay SHORT, Del Mar, CA (US)

(21) Appl. No.: 15/517,613

(22) PCT Filed: Oct. 9, 2015
(86) PCT No.: PCT/US15/54902

§ 371 Date: Apr. 7, 2017

Related U.S. Application Data
(60) Provisional application No. 62/062,270, filed on Oct. 10, 2014.

(51) Int. Cl. C07K 14/495; G01N 33/566; A61K 31/713; C12N 5/10; C07K 16/22; G01N 33/68; C12N 15/00

(52) U.S. Cl. CPC C07K 14/495; C07K 16/22; G01N 33/566; G01N 33/68; A61K 31/713; C12N 5/10; C12Q 2600/156; C12N 15/00

(57) ABSTRACT

The present invention generally relates to novel TGFα protein mutants having surprisingly superior or beneficial or different characteristics as compared to the native TGFα protein. The invention further relates to the use of the novel TGFα protein mutants in methods and kits for treatment of neurological disorders.

### FIELD OF INVENTION

The field of the invention is disorders of the central and peripheral nervous systems.

### BACKGROUND OF THE INVENTION

Neurogenesis in mammals is complete early in the postnatal period. Cells of the adult mammalian central nervous system (CNS) have little or no ability to undergo mitosis and generate new neurons. Thus, the generation of new CNS neurons in adult primates does not normally occur. This inability to produce new nerve cells in most mammals (and especially primates) may be advantageous for long-term memory retention; however, it is a distinct disadvantage when the need to replace lost neuronal cells arises due to injury or disease.

CNS disorders encompass numerous afflictions such as neurodegenerative diseases (e.g. Alzheimer's and Parkinson's), acute brain injury (e.g. stroke, head injury, cerebral palsy) and a large number of CNS dysfunctions (e.g. depression, anxiety, epilepsy, and schizophrenia). Degeneration in a brain region known as the basal ganglia can lead to diseases with various cognitive and motor symptoms, depending on the exact location. Other forms of neurological impairment can occur as a result of neural degeneration, such as cerebral palsy, or as a result of CNS trauma, such as stroke and epilepsy. In the case of Alzheimer's disease, there is a profound cellular degeneration of the forebrain and cerebral cortex. In the case of Parkinson's disease, degeneration is seen in the substantia nigra par compacta. This area normally sends dopaminergic connections to the striatum that are important in regulating movement. (Cooper and Isacson, J. Neurosci, 2004, Oct. 13: 24(41): pg. 8924-8931); Ashby et al., Neuropsychiatric Disease and Treatment 2015: 11; pg 1859-1875) Dopamine is a catecholamine neurotransmitter that is particularly important in the control of movement. The great majority of brain dopamine is found in the striatum, and contained in neurons originating from a brain stem nucleus, the substantia nigra. The death of these cells, with a consequent loss of dopamine, is responsible for the symptoms of Parkinson's disease. Other dopaminergic neurons of the brain stem innervate the limbic system and cortex and abnormalities of these systems have been implicated in schizophrenia. Therapy for Parkinson's disease has centered upon restoring dopaminergic activity to this circuit through the use of pharmaceutical compounds and/or neurotrophic factors.

Dopamine has also been shown to be important in mood, cognition, memory (e.g., working memory, episodic memory, semantic memory, procedural memory, perceptual representation memory, etc.), learning and rule application. (Ashby, et al.) Moreover, deficits in the dopaminergic pathway have been shown to be involved in disorders such as, for example, decreased mood, reduced cognition, reduced memory function, anxiety and depression. Many classical methods for treating such disorders involve administration of synthetic chemicals aimed at increasing neurotransmitter levels by inhibiting re-uptake of various neurotransmitters. Examples of such treatments are disclosed in U.S. Pat. Nos. 8,372,451, 8,461,389 and 8,796,337, all of which are incorporated herein by reference in their entirety.

Neurotrophic factors are peptides that variously support the survival, proliferation, differentiation, size, and function of nerve cells (for review, see Loughlin and Fallon, Neurotrophic Factors, Academic Press, San Diego, Calif., 1993). While the numbers of identified trophic factors, or growth factors, are ever-increasing, most can be assigned to one or another established family based upon their structure or binding affinities. Growth factors from various families, including the epidermal growth factor (EGF) family, have been demonstrated to support dopaminergic neurons of the nigrostriatal system (for review, see Hefti, J. Neurobiol. 25:1418-1435, 1994; Unsicker, Prog. Growth Factor Res. 5:73-87, 1994). EGF has been localized to areas of the developing adult forebrain and to areas of the midbrain such as the glans pallidus, ventral pallidum, entopeduncular nucleus, substantia nigra, and the Islands of Calleja. The EGF receptor was localized by immunocytochemistry to astrocytes and subpopulations of cortical and cerebellar neurons in rat brain and to neurons in human autopsy brain specimens.

Transforming growth factor-alpha (TGF-α) is a member of the EGF family that has been shown to bind the EGF receptor, stimulate the receptor's tyrosine kinase activity, and elicit similar mitogenic responses in a wide variety of cell types. TGF-α also supports the survival of mesencephalic dopamine neurons in dissociated cell culture and supports the survival of cultured central neurons. In fact, a null mutation in the TGF-α gene has been shown to reduce midbrain dopaminergic neurons in the substantia nigra. (Blunt, Nat. Neurosci. 1998, September; 1(5): p374-377)

Treatment of CNS disorders has focused not only on the administration of trophic factors, but also on the administration of stem cells to replace those neural cells lost by natural cell death, injury or disease. For example, multipotent neural stem cells that are capable of producing progeny that differentiate into neurons and glia exist in adult mammalian neural tissue. Moreover, recent studies indicate that neuronal tissue can be generated from cells derived from adult bone marrow. These studies demonstrate that transplanted adult bone marrow stem cells can differentiate into neuronal cells.

Despite recent advances in treating neurological deficits, most treatments still require the direct application of a trophic factor or infusion of stem cells to a site of injury or damage in the CNS in a subject in need of such treatment. U.S. Pat. Nos. 7,790,669, 7,795,202; 8,158,578 and US patent publication 2001/0007657 discuss methods for treating subjects for neurological diseases using members of the EGF family such as EGF and TGF-α, and are expressly incorporated herein. However, there still remains a need for additional methods of treatment. The present invention addresses such need.

### SUMMARY OF THE INVENTION

The present invention relates to the development and use of novel TGFα protein mutants having surprisingly superior activity as compared to the native human TGFα protein. The TGFα mutants possess superior properties such as increased EGFR binding affinity and/or increased EGFR phosphorylation activity, and also exhibit differences in characteristics such as hydrophobicity, soluble expression in E. coli and isoelectric points when compared to the native protein. The present invention further relates to nucleic acid molecules encoding such mutants. The present invention further relates to the use of the novel TGFα mutants in methods and compositions for treating a subject having a disease, disorder or condition of the central nervous system. The methods include administering one or more of the novel TGFα mutants, related polypeptides, fragments and mimetics thereof, useful in stimulating progenitor cell or stem cell proliferation, migration and/or differentiation.

All publications, GenBank sequences, patents and patent applications cited herein are hereby expressly incorporated by reference for all purposes. All publications, patents and patent applications, GenBank sequences, mentioned herein are incorporated herein by reference in full for the purpose of describing and disclosing the cell lines, vectors, and methodologies that are described in the publications that might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A-C. Quantitation ELISA assay for measuring hTGFα expression. 1A) Layout for the quantitative ELISA assay. 1B) OD450 values obtained from the standardized assay. 1C) Standard curve generated using the OD450 values from the table in FIG. 1B. Y-axis is the OD450 value. X-axis is the concentration of hTGFα (ng/ml).

FIG. 2A-C show the Affinity ELISA assay to determine the binding activity of hTGFα to EGFR. 2A) Layout of the affinity ELISA assay. 2B) OD450 values obtained from the assay. 2C) Standard curve generated using the OD450 values from the table in FIG. 2B. Y-axis is the OD450 value. X-axis is the concentration of hTGFα (ng/ml).

FIG. 3A-C show the Cell-based ELISA assay to determine the binding activity of hTGFα to EGFR. 3A) shows the assay layout. 3B) shows the OD450 values obtained from the cell-based assay. 3C) shows the standard curve generated using the OD450 values from the table in FIG. 3B. Y-axis is the OD450 value. X-axis is the concentration of hTGFα (ng/ml).

FIG. 4A-C show the FACS analysis to determine the binding activity of hTGFα to EGFR. 4A) Assay layout. 4B) Percentage of A431 cells bound to hTGFα. 4C. FACS profiles of A431 cells bound to hTGFα. Y-axis shows number of cells analyzed. X-axis shows fluorescence values.

FIG. 5A-D show the induction of EGFR phosphorylation by hTGFα. 5A) Assay Layout. 5B) Level of phosphorylated EGFR present in the treated cells Y Axis: OD 450 nm values of phosphorylated EGFR X Axis: Concentration of EGF (Open bars) or TGFα (Striped bars) used in the assay. 5C) Total EGFR Assay result: Level of total EGFR present in the treated cells. Y-Axis: OD450 nm values of phosphorylated EGFR. X-Axis: Concentration of EGF (Open bars) or hTGFα (Striped bars) used in assay. 5D) Normalized phosphorylated EGFR assay result. Y-Axis: OD450 nm values of phosphorylated EGFR (5B) divided by OD450 nm values of total EGFR 5(C), multiplied by 100. X-Axis: Concentration of EGF (Open bars) or hTGFα (Striped bars) used in assay.

FIG. 6 Expression levels of bacterially expressed hTGFα by SDS/PAGE analysis. Proteins expressed upon the addition of 1 mM IPTG at 37° C. for 3 hours (lanes 3 and 4), 30° C. for 6 hours (lanes 5 and 6) or 25° C. overnight (lanes 7 and 8). Lanes 1 and 2 contain supernatant (lane 1) or pellet (lane 2) from uninduced cultures. The arrow indicates hTGFα.

FIG. 7A-D show expression levels of bacterially expressed hTGFα by quantitation ELISA. 7A) Assay layout: 7B) OD 450 nm values obtained from assay. 7C) Standard curve: Y-axis: OD450 nm value. X-axis: Concentrations of TGFα (ng/ml). 7D: Concentrations (ng/ml) of hTGFα expressed in BL21 or in CHO cells estimated using standard curve.

FIG. 8A-C show binding activity of bacterially expressed hTGFα by cell-based ELISA. 8A) Assay layout. FIG. 8B) OD 450nm value FIG. 8C) Standard curve. Y-axis: OD450 nm value. X-axis: Concentrations of hTGFα (ng/ml)

FIG. 9A-B show binding activity of bacterially expressed hTGFα by FACS analysis. 9A) Assay layout. FIG. 9B) Percentage of A431 cells bound to hTGFα.

FIG. 10A-D show induction of EGFR phosphorylation by the bacterially expressed hTGFα.

10A) Assay layout. 10B) Phosphorylated EGFR Assay result: OD 450 nm values of phosphorylated EGFR. Cell Signaling kit#7240 was used to determine the level of phosphorylated EGFR present in the treated cells. 10C) Total EGFR Assay result: OD450 nm values of total EGFR present in the cells. Cell Signaling kit#7250 was use to determine the level of total EGFR present in the treated cells. 10D) Normalized phosphorylated EGFR.

FIG. 11A-B list physical and functional characteristics of CPE™ mutants of the invention. 11A) Alterations made in each CPE™ mutant and the related affinity data for the primary and first confirmation screens. 11B) Functional data for each of the CPE™ mutants constructed.

FIG. 12A-B show binding activity (using Cell-based affinity ELISA) and EGFR phosphorylation activity of selected CPE™ mutants. 12A) Cell-based affinity ELISA. X-axis: CPE™ mutant ID. Y-axis: Normalized ELISA value: OD 450nm values of CPE™ mutants divided by OD 450 nm values of wild type hTGFα. The normalized ELISA value for wild type hTGFα is 1 (solid horizontal line) White bars: ELISA data from the first confirmation. Black bars: ELISA data from the second confirmation. 12B) EGFR phosphorylation. X-axis: CPE™ mutant ID. Y-axis: Normalized phosphorylated EGFR Assay result: OD 450 nm values of phosphorylated EGFR (B) divided by OD450 nm values of total EGFR (C) by 100.

FIG. 13 Binding activity of recombinantly expressed, CPS™ mutants using Cell-based affinity ELISA. OD450 nm value of the reactions was measured with a Molecular Device SPECTRAmax Plus plate reader. *Note: Some of the CPS™ mutants were tested using less than 25 ng/ml (see CPS™ map, FIG. 16). X-axis: CPS™ mutants. Y-axis: OD 450 nm values of the ELISA reaction.

FIG. 14 EGFR phosphorylation activity of the CPS™ mutants. X-axis: CPS™ mutants. Y-axis: Normalized phosphorylated EGFR Assay result: OD 450 nm values of phosphorylated EGFR divided by OD450 nm values of total EGFR, multiplied by 100.

FIG. 15 Binding activity of selected CPS™ mutants using the Cell-based titration affinity ELISA. X-axis: CPS™ mutant ID or wild type hTGFα at the indicated concentration. Y-axis: OD 450 nm values ELISA reactions.

FIG. 16 shows the amino acid sequence of CPS™ mutants of the invention. It also shows some physical characteristics of the CPS™ mutants.

FIG. 17 shows physical and functional characteristics of CPS™ mutants of the invention.

### DETAILED DESCRIPTION OF THE INVENTION

Described herein are novel TGFα protein mutants or variants having surprisingly superior or beneficial or different characteristics as compared to the native TGFα protein. The development, preparation and characterization of the novel mutants are described in detail in the Examples below. The novel TGFα mutants described herein exhibit equal, or greater, binding affinity to EGFR and/or EGFR phosphorylation activity as compared with the wild type TGFα. Additionally, the novel mutants may exhibit different characteristics, such as hydrophobicity, isoelectric points and soluble expression in E. coli as compared to the wild type TGFα.

As described in detail in Example 2, a Comprehensive Positional Evolution (CPE™) library of BAP060-hTGFα-B (amino acid sequence represented by SEQ ID NO:145) was constructed with 15 amino acid variants created at each position except for position 30 which has 14 amino acid variants. The CPE™ library contained 660 BAP060-hTGFα-B mutants. The mutants were expressed and their activity measured using the assays described in Example 1. Out of these, 7 CPE™ hits based on their superior or different characteristics compared to the wild type hTGFα were identified and a Combinatorial Protein Synthesis (CPS™) library was constructed which contained all possible combinations of the 7 CPE™ mutations. These mutants were expressed and those with superior or different characteristics compared to the wild type TGFα were identified.

Thus, the present invention provides novel TGFα mutant proteins which have superior or beneficial or different characteristics compared to the wild type TGFα, and related polypeptides, fragments and mimetics thereof, and their amino acid sequences. The present invention also provides nucleic acid molecules encoding the novel TGFα mutant proteins and their nucleic acid sequences. The present invention further provides expression systems including without limitation, plasmids, vectors, recombinant viruses or cells that express the novel TGFα mutant proteins.

The present invention further provides use of the novel TGFα mutants in therapeutic methods for generating, or regenerating, or modulating, neuronal tissues and their functions in vitro and in vivo and for ameliorating neurological deficits, including without limitation, inherited disorders, traumas, infections, autoimmune diseases and the like.

The novel TGFα mutants of the present invention may be used in methods of treating injuries or damage to the CNS that do not require the direct administration of a trophic factor to a site of injury or the introduction of stem cells into the subject being treated; and/or in methods for promoting neurogenesis or modulating neural activity of injured, damaged or diseased central nervous system (CNS) tissue through the peripheral administration of a trophic factor.

The invention provides methods comprising administration of a TGF-α mutant, or a related polypeptide, functional fragment, or a mimetic thereof, to a subject to modulate or promote neurogenesis or neural activity, By providing various ways to generate new glial and neuronal cells from endogenous progenitor cells, the invention also provides methods for inducing regeneration of tissues and neurological function. The invention provides methods for attracting a progenitor cell, or progeny thereof, to a site of injury or damage in the central nervous system of a subject by peripherally administering a therapeutically effective amount of aTGF-α mutant, or a related polypeptide, functional fragment, or a mimetic thereof, to the subject. Thus, the invention features methods for generating, or regenerating, or modulating, neuronal tissues and their functions in vivo and for ameliorating neurological deficits, including inherited disorders, traumas, infections, autoimmune diseases and the like.

In one aspect, the invention provides a method for modulating neurogenesis at a site of injury or damage in the central nervous system of a subject by peripherally administering a therapeutically-effective amount of a TGF-α mutant, or related polypeptide, functional fragment, or a mimetic thereof, to the subject. While the invention is not limited by any particular mechanism of action, the administration can induce the proliferation, migration, differentiation (including dedifferentiation or redifferentiation) of a cell, such as a progenitor cell, or progeny thereof, to the site of injury or damage in the central nervous system. In an alternative aspect, the administration induces the proliferation, migration, or differentiation (including dedifferentiation or redifferentiation) of a cell, such as a progenitor cell, or progeny thereof, at or near the site of injury or damage in the central nervous system. In alternative aspects, the progenitor cell, and/or its progeny, express polypeptides comprising a CD34+, a Sca-1+, or a CD 117+(c-kit+) polypeptide. In alternative aspects, the methods induce differentiation, or de-differentiation, of a cell, and the resultant cell expresses polypeptides comprising a CD34+, a Sca-1+, or a CD 117+(c-kit+) polypeptide. In alternative aspects of the methods of the invention, the progenitor cell is a pre-hematopoietic stem cell, a hematopoietic stem cell, an endothelial cell and a perivascular cell. However, the progenitor cell can be any cell at any level of development or differentiation that differentiates, e.g., de-differentiates or re-differentiates, in response to the methods of the invention. In alternative aspects of the methods of the invention, the central nervous system injury or damage is a neurodegenerative disease, a traumatic injury, a neurotoxic injury, an ischemia (e.g., from an ischemic event), a developmental disorder, a disorder affecting vision, an injury or disease of the spinal cord, a demyelinating disease, an autoimmune disease, an infection and an inflammatory disease. However, it is understood that any neurological disorder benefited by the peripheral administration of a trophic factor that induces an endogenous progenitor cell, or progeny thereof, to directly or indirectly result in the repopulation of damaged neuronal tissue in the central nervous system is encompassed by the present invention. In one aspect, the disorder affecting vision effects or is related to the retina. In alternative aspects, the neurodegenerative disease is Alzheimer's disease, Huntington's disease, amyotrophic lateral sclerosis (ALS) and Parkinson's disease. In one aspect, the injury or damage results from a neoplastic lesion. In one aspect, the injury or damage results from radiation or chemotherapeutic treatment of the central nervous system.

In alternative aspects of the methods of the invention, the peripheral administration comprises subcutaneous administration, intravenous administration, intradermal administration, intramuscular injection, topical administration and oral administration. In one aspect, the method further comprises mechanically disrupting tissue in the CNS, thereby directing translocation of the progenitor cell, or progeny thereof. In one aspect, the method further comprises neurochemically blocking the activity of cells in the central nervous system, thereby directing migration of the progenitor cell, or progeny thereof.

The invention further provides a method for modulating neurogenesis or neural activity at a site of injury or damage in the central nervous system in a subject by peripherally administering a therapeutically effective amount of a nucleic acid comprising a nucleotide sequence encoding a TGF-α mutant, or related polypeptide, functional fragment, or a mimetic thereof. In one aspect, the nucleic acid sequence is present in an expression vector, including, e.g., naked DNA, plasmids, recombinant viruses, and the like. In one aspect, the nucleic acid sequence is operably linked to an expression control sequence, including, e.g., promoters, enhancers and the like. In one aspect, the expression induces the proliferation, migration, or differentiation of a progenitor cell, or progeny thereof, into, at, or near the site of injury or damage resulting in modulation of neurogenesis or neural activity in the subject. In one aspect, the method further comprises neurochemically blocking the activity of cells in the CNS, thereby directing migration of the progenitor cell, or progeny thereof.

The invention further provides a method for treating, regenerating or repairing a central nervous system tissue of a subject in vivo, by contacting the tissue with a TGF-α mutant, or related polypeptide, functional fragment, or a mimetic thereof, prior to, contemporaneously with, or subsequent to a tissue injury or damage in an amount effective to induce the proliferation, migration, or differentiation of a progenitor cell at or near the site of injury, thereby treating, regenerating or repairing the tissue. The invention further provides a method for attracting a progenitor cell (e.g., a hematopoietic progenitor cell, a progenitor cell in a vascular endothelium or perivascular tissue, or a progenitor cell from bone marrow or other organ), or a progeny thereof, to a site of injury or damage in the central nervous system comprising administering a TGF-α mutant, or related polypeptide, functional fragment, or a mimetic thereof, in a pharmaceutically acceptable carrier, to a site not associated with the site of injury or damage, sufficient to attract the cell to the site. In alternative aspects, the progenitor cell is a non-neuronal progenitor cell, a pre-hematopoietic stem cell, a hematopoietic stem cell, an endothelial cell or is derived from a vascular endothelium and a perivascular cell.

The invention further provides a method for ameliorating a neurological deficit in a subject (e.g., treating a subject having a neurological deficit), the method comprising contacting a progenitor cell of the subject in vivo with a TGF-α mutant, or related polypeptide, functional fragment, or a mimetic thereof, wherein the contacting results in a signal transduction sufficient to mobilize the progenitor cell, or progeny thereof, to a region of the central nervous system of the patient, wherein the progenitor cell differentiates into a neuronal cell and functions in a manner sufficient to ameliorate (e.g., reduce) the neurological deficit. In one aspect, the progenitor cell expresses polypeptides comprising a CD34+, a Sca-1+, or a CD 117+(c-kit+) polypeptide.

The invention further provides a method for expansion of a progenitor cell comprising contacting the cell in vivo with an amount of a TGF-α mutant, or related polypeptide, functional fragment, or a mimetic thereof, effective to promote progenitor cell proliferation in vivo. The invention further provides a method of directing the differentiation of an endogenous progenitor cell in a subject, the method comprising contacting the progenitor cell with a TGF-α mutant, or related polypeptide, functional fragment, or a mimetic thereof, whereby the progenitor cell differentiates at or near the site of damage or injury to a neuronal tissue and wherein the cell acquires the phenotypic characteristics of the differentiated cells of the neuronal tissue.

The invention provides a method for prophylactically ameliorating an effect of an injury in the central nervous system or the peripheral nervous system of a subject comprising: peripherally administering an effective amount of a TGF-α polypeptide to the subject, thereby prophylactically ameliorating the effect of an injury in the central nervous system or the peripheral nervous system of the subject.

All publications, GenBank sequences, patents and patent applications cited herein are hereby expressly incorporated by reference for all purposes. All publications, patents and patent applications, GenBank sequences, mentioned herein are incorporated herein by reference in full for the purpose of describing and disclosing the cell lines, vectors, and methodologies that are described in the publications that might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention. The details of one or more embodiments of the invention are set forth in the accompanying drawings and the description below, Other features, objects, and advantages of the invention will be apparent from the description and drawings, and from the claims.

As used herein, “endothelium” or “endothelial cells” constitute the layer of cells that line the cavities of the heart and of the blood and lymph vessels, and the serous cavities of the body, originating from the mesoderm. Endothelial cells lining a blood vessel are linked to each other laterally by occluding junctions which may either form continuous belts to create permeability barriers (zonulae occludentes) and hold the cells together or in some cases discontinuous, spot-like contacts (maculae occludentes), allowing some leakage between cells. Endotheliocytes are also linked to each other, and often to other extravascular cells, by gap (communicating) junctions facilitating cooperation between cells connected in this way. Endothelium has important metabolic functions and a variety of enzyme systems have been located within or at the surfaces of endothelial cells. For example, vascular endothelial cells are required in the process of angiogenesis. Angiogenesis occurs during tissue regeneration and is facilitated by the migration and proliferation of existing endothelium and by the differentiation of connective tissue progenitor cells, such as, for example, pluripotent mesenchymal stem cells.

As used herein, an “SVZ progenitor cell” is a multipotential stem cell originating in the subventricular zone (SVZ) of the brain. As used herein, a “perivascular,” cell is a cell that is situated around a blood vessel. Perivascular cells are in close proximity to the structure of the blood vessel wall and as such are capable of receiving chemical signals initiated from the lumen of the blood vessel and transduced through the wall of the blood vessel. Thus, a peripherally administered trophic factor that normally does not cross the blood brain barrier (BBB), such as TGF-α, can still influence the generation of neural tissue by acting on cells positioned near a cerebral blood vessel such as an SVZ progenitor cell or perivascular cell, As used herein, a “peripherally” administered trophic factor is administered at a site not directly associated with the injury or damage to be treated in the CNS. The method of the invention does not require the direct contact of the trophic factor with the injured or damaged neuronal tissue. Thus, a peripherally administered trophic factor can modulate neurogenesis or neural activity at a site of injury or damage in the central nervous system of a subject without having to cross the blood brain harrier (BBB) through signal transduction.

As used herein, a “progenitor cell” includes any non-terminally differentiated cell such as, for example, a stem cell including an adult stem cell. A progenitor cell has specific biochemical properties, may or may not divide, and can be triggered to adopt a different differentiation state but not necessarily a fully differentiated state, by responding to specific developmental signals. Encompassed within the definition of “progenitor cell” is a cell that has de-differentiated into a less differentiated cell and now possesses pluripotential capabilities. The present study envisions that those cells associated with a cerebral blood vessel, including endothelial cells, perivascular cells, circulating HSCs and/or SVZ progenitor cells possess pluripotential capabilities. Such cells are capable of responding to microenvironmental signals generated by both TGF-α and signals elaborated by the denervated neurons, and/or their supporting cells (astrocytes, oligodendrocytes and microglia), in a manner suitable for the generation of neural tissue by the method of the invention.

The terms “progenitor cell” and “stem cell” are often used interchangeably herein. Stem cell or progenitor cells that can be stimulated in vivo to proliferate, migrate and/or differentiate when contacted by a TGF-α polypeptide, a related polypeptide, mimetic, or functional fragment thereof having TGF-α activity, include adult stem cells and precursor cells. Such stem cells and precursor cells include, for example, cells of hematopoietic tissue, neuronal tissue, perivascular tissue and endothelial cells.

Hematopoietic stem cells (HSCs) are the formative pluripotential blast cells found inter alia in bone marrow and peripheral blood that are capable of differentiating into the specific types of hematopoietic or blood cells, such as erythrocytes, lymphocytes, macrophages, and megakaryocytes After mobilization of HSCs from bone marrow by administration of certain factors, such as G-CSF and W-CSF and subsequent recovery from peripheral blood, HSCs have also come to be referred to as peripheral blood progenitor cells (PBPCs).

Mesenchymal stem cells (MSCs) are the formative pluripotential blast cells found inter alia in bone marrow, blood, dermis and periosteum that are capable of differentiating into more than one specific type of mesenchymal or connective tissue (e.g., adipose, osseous, stroma, cartilaginous, elastic, and fibrous connective tissues) depending upon various influences from bioactive factors, such as cytokines. The potential to differentiate into cells such as osteoblasts and chondrocytes is retained after isolation and expansion in culture; differentiation occurs when the cells are induced in vitro under specific conditions or placed in vivo at or near the site of damaged tissue.

In addition to hematopoietic stem cells, bone marrow contains stem-like precursors for non-hematopoietic cells, such as osteoblasts, chondrocytes, adipocytes and myoblasts (Owen et al., 1988, in Cell and Molecular Biology of Vertebrate Hard Tissues, Ciba. Foundation Symposium 136, Chichester, UK, pp. 42-60; Caplan, 1991, J. Orthop. Res. 9:641-650; Prockop, 1997, Science 276:71-74). Non-hematopoietic precursors of the bone marrow have been variously referred to as colony-forming-unit-fibroblasts, mesenchymal stem cells, and marrow stromal cells (MSCs). As used herein, “stromal cells”, “colony forming fibroblasts”, “marrow stromal cells”, “adherent cells” and “MSCs” are used interchangeably and are meant to refer to the small fraction of cells in bone marrow which can serve as stem cell-like like precursors of osteocytes, chondrocytes, and adipocytes.

As used herein, a “pluripotent cell” is a cell that may be induced to differentiate, in vivo or in vitro, into at least two different cell types. These cell types may themselves be pluripotent, and capable of differentiating in turn into further cell types, or they may be terminally differentiated, that is, incapable of differentiating beyond their actual state.

Pluripotent cells include totipotent cells, which are capable of differentiating along any chosen developmental pathway. For example, embryonal stem cells (Thomson et al., Science, 282:1145, 1998) are totipotent stem cells. Pluripotent cells also include other, tissue-specific stem cells, such as hematopoietic stem cells, mesenchymal stem cells, neuronal stem cells, neuroectodermal ectodermal cells, and endodermal cells, for example, gut endodermal cells and mesodermal stem cells which have the ability to give muscle or skeletal components, dermal components, such as skin or hair, blood cells, etc. “Developmental pathway” refers to a common cell fate that can be traced from a particular precursor cell. Progenitor cells are more primitive, i.e., less fated to a particular developmental pathway than mesenchymal stem cells or hematopoietic stem cells.

A “partially committed” cell is a cell type that is no longer totipotent but remains pluripotent. For example, an SVZ stem cell is partially differentiated but still capable of further differentiating into various neuronal tissue types. Similarly, non-terminally differentiated endothelial cells can be stimulated to differentiate into neuronal tissue.

A pluripotent progenitor cell can be responsive to a cell proliferation-modulating agent. As used herein, a “cell proliferation-modulating agent” is any agent that can promote or inhibit cell growth or differentiation.

As used herein, the term “trophic factor” refers to compounds with trophic actions that promote and/or control proliferation, differentiation, migration, and survival (sometimes even the death) of their target cells, and/or protect them from injury. Such factors include cytokines, neurotrophins, epidermal growth factor, fibroblast growth factor, platelet-derived growth factor, insulin-like growth factors, ciliary neurotrophic factor and related molecules, glial-derived growth factor and related molecules, schwanoma-derived growth factor, glial growth factor, stiatal-derived neuronotrophic factor, platelet-derived growth factor, hepatocyte growth factor, scatter factor (HGF-SF), transforming growth factor-beta and related molecules, neurotransmitters, and hormones. Those of skill in the art will readily recognize additional trophic factors which can be employed in the present invention (see, e.g., Lenfant et al., Growth Factors of the Vascular and Nervous Systems: Functional Characterization and Biotechnology: international Symposium on Biotechnology of Grow (S. Karger Publishing, 1992).

The term “growth factor,” as used herein, includes those molecules that function as growth simulators (mitogens) or as growth inhibitors (sometimes referred to as negative growth factors). Growth factors are also known to stimulate cell migration (e.g., mitogenic cytokines), function as chemotactic agents, inhibit cell migration or invasion of tumor cells, modulate differentiated functions of cells, be involved in apoptosis, and promote survival of cells. Such factors can be secreted as diffusible factors and can also exist in membrane-anchored forms. They can, therefore, act in an autocrine, paracrine, juxtacrine, or retrocrine manner, A cytokine is one type of growth factor. A “cytokine” is polypeptide that acts as a humoral regulator at nano-to-picomolar concentrations and which, either under normal or pathological conditions, can modulate the functional activities of individual cells and tissues. A cytokine can mediate interactions between cells directly and/or can regulate processes taking place in the extracellular environment. Cytokines comprise interleukins, lymphokines, monokines, interferons, colony-stimulating factors, and chemokines, in addition to a variety of other proteins.

Growth factors further include epidermal growth factors (EGFs), transforming growth factors (TGFs), platelet-derived growth factors (PDGFs), fibroblast growth factors (FGFs), hepatocyte growth factors (HGFs), hematopoietic growth factors (HeGFs), tumor necrosis factor (TNF-α), platelet-derived endothelial cell growth factor (PD-ECGF), insulin-like growth factor (IGF), interleukin-8, growth hormone, angiopoietin, vascular endothelial growth factor (VEGF), acidic and basic fibroblast growth factors (FGFs), transforming growth factor a (TGF-α or TGF-α), and CYR 61 (Babic et at, Proc. Natl. Acad. Sci. USA, 95:6355, 1998; Kireeva et al., Mol. Cell. Biol., 16:1326, 1996). Such factors further include insulin, IGF-I, IGF-II, nerve growth factor, NGF receptor, EGF, TGF-α, EGF receptor, neu, TGF-β1, TGF-β2, TGF-β3, inhibin α, inhibin β, Müillerian inhibitory substance, TNF-α/β, TNF-receptor (type 1), TNF-receptor (type 2), PDGF A-chain, PDGF B-chain, PDGF receptor α, PDGF receptor β, a-FGF, b-FGF, wnt-2, hst/ks3, hepatocyte growth factor, HGF receptor (c-met), IL-1α/β, (α-chains) IL-2, IL-3, IL-4 IL-5, IL-7, IL-9, IL-12A (p35), IL-12B (p40), Interleukin 1 (type 1), Interleukin-2α, Interleukin-2β, Interleukin-4, Interleukin-5α, Interleukin-6, Interleukin-7, M-CSF (also called CSF-1), M-CSF receptor (c-fms), GM-CSF, GM-CSF receptor α, GM-CSF receptor β, G-CSF, G-CSF receptor, stem cell factor, SCF receptor (c-kit), Erythropoietin (epo), epo receptor, and Leukemia inhibitory factor. Each of these molecules has been shown to induce cell proliferation, cell growth or differentiation in vivo. Other similar molecules that display cell growth or differentiation modulating activity are the heparin binding growth factors (HBGFs).

A “polypeptide” or protein refers to a polymer in which the monomers are amino acid residues that are joined together through amide bonds. When the amino acids are α-amino acids, either the L-optical isomer or the D-optical isomer can be used, the L-isomers being typical. A TGF-α mutant, a related polypeptide, mimetic, or functional fragment thereof is intended to encompass an amino acid sequence, including modified sequences such as glycoproteins, which exhibit the mutant TGF-α activity. The molecules (e.g., polypeptides) of the invention encompass amino acid sequences of TGF-α mutants as well as polypeptides that have structural and/or functional characteristics of TGF-α mutants. The polypeptides for use in the methods of the invention are intended to cover substantially purified naturally occurring proteins, as well as those that are recombinantly synthesized, semi-synthetically synthesized, or synthetically synthesized.

The term “TGFα” includes any known TGFα polypeptide; a fragment of a TGF-α polypeptide; a variant of a known TGFα polypeptide; a fusion polypeptide comprising a TGFα polypeptide; and a TGFα mimetic. Variants include polypeptides having substantial amino acid sequence identity to a known TGFα polypeptide; polypeptides comprising one or more conservative amino acid changes compared to a known TGFα polypeptide; and the like. Examples of known TGFα polypeptides include a TGFα polypeptide having amino acids 17-66 of the amino acid sequence provided in GenBank Accession No. P01135; and a TGFα polypeptide having amino acids 16-65 of the amino acid sequence provided in GenBank Accession No. P01134. For use in the methods of the invention, a TGFα polypeptide is biologically active, e.g., it retains the ability to induce differentiation and/or migration and/or proliferation of progenitor cells into a site of injury or damage in the CNS. Whether a TGFα polypeptide induces differentiation and/or migration and/or proliferation of progenitor cells into a site of injury or damage in the CNS can be determined using the methods described herein, and the methods described in WO 99/06060. In addition, a TGF-α or related polypeptide can occur in at least two different conformations wherein both conformations have the same or substantially the same amino acid sequence but have different three dimensional structures so long as the have a biological activity related to TGF-α. Methods of using polypeptide or protein fragments of TGF-α are also encompassed by the invention. Fragments can have the same or substantially the same amino acid sequence as the naturally occurring protein. A polypeptide or peptide having substantially the same sequence means that an amino acid sequence is largely, but not entirely, the same, but retains a functional activity of the sequence to which it is related.

In general, polypeptides useful in the method of the present invention include peptides, or full length protein, that contain substitutions, deletions, or insertions into the protein backbone, that would still have from about 50% to about 70%, from about 70% to about 80%, from about 80% to about 90%, from about 90% to about 95%, from about 95% to about 98%, or higher, amino acid sequence identity to the original protein over the corresponding portion. A yet greater degree of departure from identity is allowed if like-amino acids, i.e. conservative amino acid substitutions, do not count as a change in the sequence. A TGF-α polypeptide fragment retains a biological activity associated with TGF-α.

Sequence identity (homology) to TGF-α polypeptide can be used to determine if is polypeptide is a composition used in a method of the invention; it can be measured using standard sequence analysis software (e.g., Sequence Analysis Software Package of the Genetics Computer Group, University of Wisconsin Biotechnology Center, 1710 University Avenue, Madison, Wis. 53705; also see Ausubel, et al., supra). Such procedures and algorithms include, for example, a BLAST program (Basic Local Alignment Search Tool at the National Center for Biological Information), ALIGN, AMAS (Analysis of :Multiply Aligned. Sequences), AMPS (Protein Multiple Sequence Alignment), ASSET (Aligned. Segment Statistical Evaluation Tool), BANDS, BESTSCOR, BIOSCAN (Biological Sequence Comparative Analysis Node), BLIMPS (BLocks IMProved Searcher), FASTA, intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF.

A polypeptide may be substantially related but for a conservative variation, such polypeptides being encompassed by the invention. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine. The term “conservative variation” also includes the use of a substituted amino acid in place of an unsubstituted parent amino acid provided that antibodies raised to the substituted polypeptide also immunoreact with the unsubstituted polypeptide.

Modifications and substitutions are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra (D)-amino acids, glycosylation sites, cytosine for specific disulfide bridge formation. The modified peptides can be chemically synthesized, or the isolated gene can be site-directed mutagenized, or a synthetic gene can be synthesized and expressed in a subject such that a neurological deficit is treated.

Solid-phase chemical peptide synthesis methods can also be used to synthesize polypeptide or fragments of polypeptides can be used in the present methods. Such method of synthesis have been known in the art since the early 1960's (Merrifield, R. B., J. Am. Chem. Soc., 85, 2149-2154 (1963) (See also Stewart, J. M. and Young, J. D., Solid. Phase Peptide Synthesis, 2 ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81, 3998 (1984) and provide for synthesizing peptides upon the tips of a multitude of “rods” or C6pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod and pin tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 43 1 A automated peptide synthesizer.

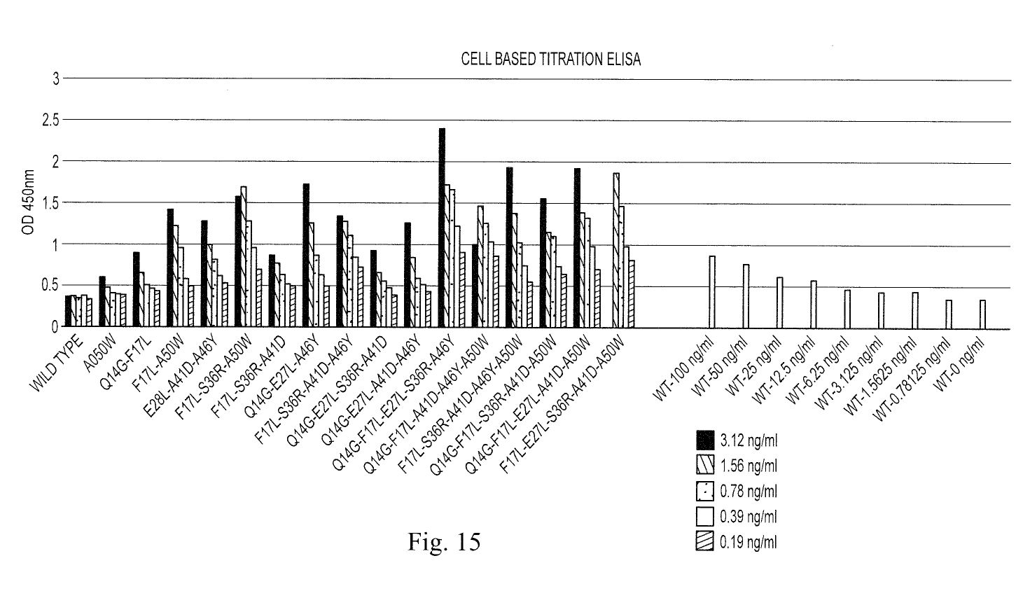

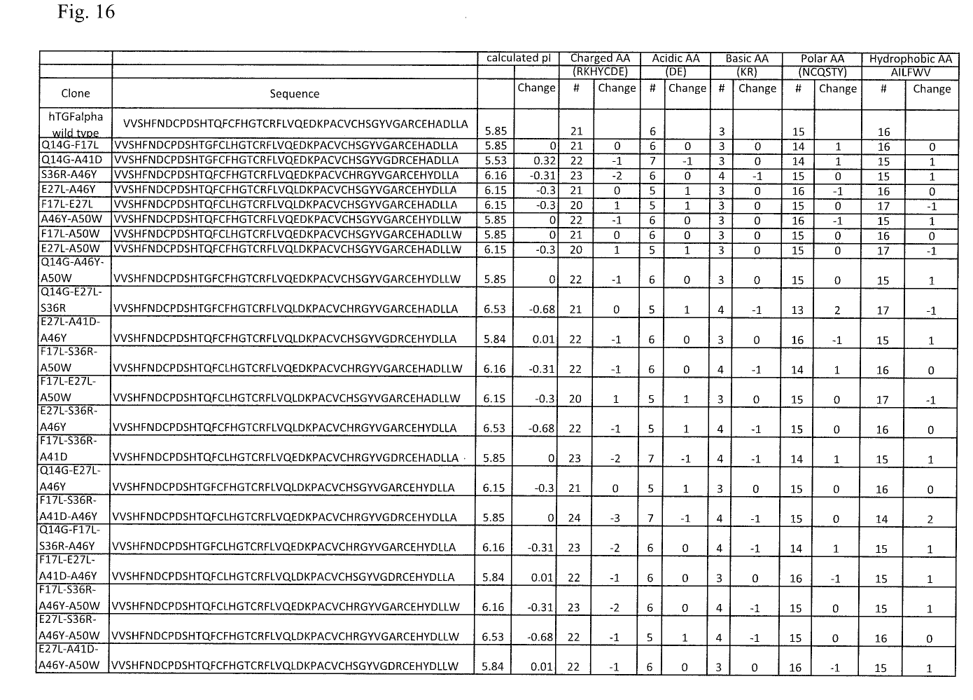

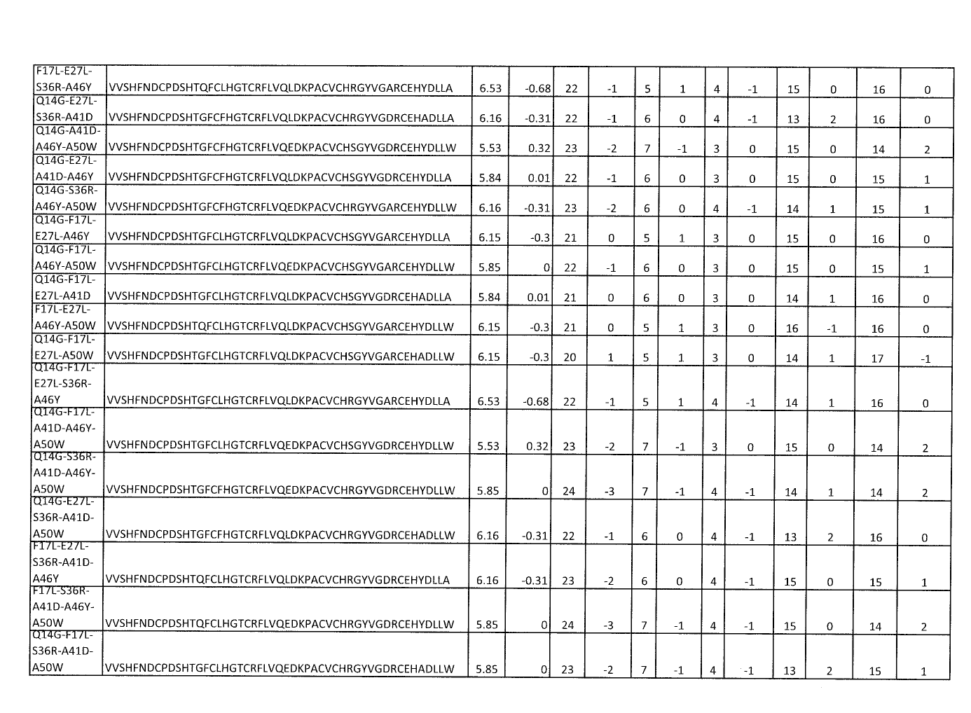

In one aspect, the invention provides novel, mutant TGFα proteins that have superior or beneficial or different characteristics compared to the wild-type TGFα protein. According to the present invention, such mutants are synthetic, meaning they are made by the hand of man. Any mutation can be made to the wild-type TGFα protein sequence, as long as the mutant protein has characteristics that are superior, beneficial, different characteristics compared to the wild-type TGFα protein. Preferred mutations are disclosed herein, as are locations at which to make such mutations. In one aspect, mutant TGFα proteins of the invention comprise mutations at one or more location disclosed herein at which a mutation can be made. One example of a useful mutation is a substitution mutation. In one aspect, mutant TGFα proteins of the invention comprise a substitution mutation at one or more location disclosed herein at which a mutation can be made. In one aspect, mutant TGFα proteins of the invention comprise one or more of the mutations disclosed herein. In one aspect, mutant TGFα proteins of the invention comprise an amino acid mutation at one or more amino acid positions corresponding to the amino acid positions listed in Table 3. In one aspect, mutant TGFα proteins of the invention comprise an amino acid mutation at one or more amino acid positions corresponding to amino acid positions 14, 17, 27, 36, 41, 45 and 50 of a wild-type TGFα. In one aspect, mutant TGFα proteins of the invention comprise an amino acid sequence selected from the sequences shown in Table 2 or the sequences listed in FIG. 16. In one aspect, mutant TGFα proteins of the invention comprise an amino acid sequence selected from the group consisting of SEQ ID NO:2-SEQ ID NO:143.

The present invention also provides variants of the novel, mutant TGFα proteins disclosed herein. According to the present invention, variants of the mutant TGFα proteins of the invention are mutant TGFα proteins disclosed herein, the sequences of which have been further modified. In preferred embodiments, variants of mutant TGFα proteins of the invention comprise the mutations of the starting mutant TGFα proteins (e.g., a mutation at one or more amino acid positions corresponding to the amino acid positions listed in Table 3 (e.g., amino acid positions 14, 17, 27, 36, 41, 45 and 50 of a wild-type TGFα)). However, the remaining amino acid sequence (i.e., the amino acids in the mutant TGFα protein other than the amino acids at positions corresponding to amino acid positions 14, 17, 27, 36, 41, 45 and 50 of a wild-type TGFα) is further mutated to generate a final variant having improved activity or characteristics. In one embodiment, the remaining amino acid sequences are mutated so that they are at least 80%, at least 85%, at least 90%, at least 92%, at least 94%, at least 95% or at least 97% identical to the corresponding sequences in the starting mutant TGFα protein. In preferred embodiments, variants mutant TGFα proteins have equal, or improved, activity (e.g., binding affinity, induction of phosphorylation, etc.) or characteristics (e.g., solubility, stability, etc) compared to the starting mutant TGFα protein. To illustrate further what is meant by a variant, a starting mutant TGFα protein could comprise, for example, SEQ ID NO:125, which has mutations at positions 14, 17, 27 and 41. This starting mutant TGFα protein would then be further mutated at locations other than positions 14, 17, 27 or 41 and tested for improved activity or characteristics. In one embodiment, a variant of a mutant TGFα protein of the invention comprises one or more mutations from a sequence selected from the group consisting of SEQ ID NO:2-SEQ ID NO:143, wherein the remaining sequences in the variant are at least 80%, at least 85%, at least 90%, at least 92%, at least 94%, at least 95% or at least 97% identical to the corresponding sequences in the starting mutant TGFα protein.

TGFα stimulates differentiation and/or proliferation and/or migration of stem cells of neural origin to sites or lesions in a neurological deficit For example, Parkinson's Disease is characterized by resting tremor, rigidity, inability to initiate movement (akinesia) and slowness of movement (bradykinesia). The motor deficits are associated with progressive degeneration of the dopaninergic innervation to the nucleus accumbens and degeneration of noradrenergic cells of the locus ceruleus and serotonergic neurons of the raphe. Up to 80% of nigral dopamine neurons can be lost before significant motor deficits are manifest. TGF-α, when infused into rat brains over a period of time (e.g., days or weeks), is useful for the treatment of neurodegenerative disorders. Intracerebroventricular (ICV) or intrastriatal infusions of TGF-α over a period of 18 days induced neuronal stem cell proliferation, but degenerating, damaged or otherwise abnormal cells are present to facilitate migration of the neuronal stem cells to a site of injury on a scale sufficient to impact recovery from an associated neurological deficit (see PCT publications WO 99/06060 and WO 01/12127, incorporated herein by reference in their entirety). Forebrain neural stem cells migrate and affect treatment and recovery from a neurological deficit disorder including, for example, Parkinson's Disease, Huntington's Disease, Alzheimer's Disease and the like.

Previous studies reported in U.S. Pat. No. 7,795,202 provide evidence that TGF-α, administered by a peripheral route, promotes neurogenesis of the central nervous system (CNS) and ameliorates abnormal motor behavior in an animal model of Parkinson's disease. Analysis of the brains of TGF-α-infused animals reveal the presence of recently generated neurons in multiple areas of the forebrain and midbrain, a few of which show terminal differentiation into dopamine (DA) producing neurons. As previously noted, TGF-α is unable to cross the BBB and typically accumulates within the cerebral vasculature. 6-OHDA, used in the present study to induce DA cell death in the SN, has been reported to disrupt the BBB but this disruption is limited to the mesencephalic areas and cerebral cortex in the vicinity of the needle tract, and not more rostral areas. Thus, it appears unlikely that TGF-α gains access to the more rostral cerebral parenchyma through the midbrain disruption of the BBB caused by 6-OHDA. For example, in the prior study TGF-α was delivered via an intravascular route from a peripheral source. Accordingly, all cerebral vessels are perfused uniformly thereby diminishing the likelihood of a unidirectional influence imposed by intracranial placement of infusion cannulae. It is thus unlikely that the clustering of newly generated cells around blood vessels only in the denervated areas represents a chemoattractive or tropic response to intravascular growth factor, as this would be expected to result in a more widespread distribution of such cells around all cerebral vessels.

As used herein, a “nucleic acid” (used interchangeably with the term “polynucleotide”) refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, fur example, a recombinant DNA which is incorporated into a vector, into an autonomously replicating plasmid or virus; or into the genomic DNA of a prokaryote or eukaryote, or which exists as a separate molecule (e.g., a cDNA) independent of other sequences. Nucleic acids suitable for use in the methods of the invention can be ribonucleotides, deoxyribonucleotides, or modified forms of either nucleotide. In addition, the polynucleotide sequence involved in producing a polypeptide chain can include regions preceding and following the coding region (leader and trailer) as well as intervening sequences (introns) between individual coding segments (exons) depending upon the source of the polynucleotide sequence. The term “polynucleotide(s)” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. Nucleic acids can be created which encode a fusion protein (e.g., a TGF-α polypeptide and another polypeptide, such as a targeting, sequence) and can be operatively linked to expression control sequences. “Operatively-linked” refers to a juxtaposition wherein the components so described are in a relationship permitting them to function in their intended manner. For example, a coding sequence is “operably linked” to another coding sequence when RNA polymerase ail transcribe the two coding sequences into a single mRNA, which is then translated into a single polypeptide having amino acids derived from both coding sequences. The coding sequences need not be contiguous to one another so long as the expressed sequences ultimately process to produce the desired protein. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences.

By “promoter” is meant minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the of a polynucleotide sequence. Both constitutive and inducible promoters are included in the invention (see e.g., Bitter et al., Methods in Enzymology 153:516-544, 1987). For example, when cloning in mammalian cell systems, promoters derived from the genome of mammalian cells (e.g., metallothionein promoter) or from mammalian viruses (e.g., the retro-virus, long terminal repeat; the adenovirus late promoter; the vaccinia virus 7.5K promoter) may be used. Promoters produced by recombinant DNA or synthetic techniques may also be used to provide for transcription of the nucleic acid sequences of the invention. Tissue-specific regulatory elements may be used, including, for example, regulatory elements from genes or viruses that are differentially expressed in different tissues. Examples of tissue-specific regulatory elements include, tissue-specific promoters, such as milk-specific (whey), pancreatic (insulin or elastase), actin promoter in smooth muscle cells or neuronal (myelin basic protein) promoters such as GFAP (specific for glial cells; see also U.S. Pat. No. 6,066,7260). Tissue specific promoters include the 5′ or 3′ flanking sequences of the beta-globin, elastase, a-fetoprotein, α-A crystalline, an erythroid specific transcriptional element and insulin genes (Yee, et al., Proc. Natl. Acad. Sci., U.S.A. 86:5873-5877, 1989; Swift, et al., Cell 38:639, 1984; Storb et al., Nature (Lond.) 310:238; Grosscheldl et al., Cell 41:885, 1985; Shani, Nature (Lond) 314:238, 1985; and Chada et al, Nature (Lond), 1985).

A recombinant expression vector includes a polynucleotide sequence encoding a TGF-α polypeptide, related polypeptides, fragments or mimetics thereof. The expression vector typically contains an origin of replication, a promoter, as well as specific genes that allow phenotypic selection of the transformed cells. Vectors suitable for use in the invention include, but are not limited to the pMSXND expression vector for expression in mammalian cells (Lee and Nathans, J. Biol. Chem. 263:3521, 1988). Mammalian cell systems which utilize recombinant viruses or viral elements to direct expression may be engineered. For example, when using adenovirus expression vectors, a polynucleotide encoding a TGF-α polypeptide, a TGF-α related polypeptide, mimetic, or a fragment having TGF-α activity may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a TGF-α polypeptide or fragment thereof in infected hosts. Direct in vivo gene transfer has been attempted with formulations of DNA trapped in liposomes (Ledley et al., J. Pediatrics 1 10: 1, 1987); or in proteoliposomes that contain viral envelope receptor proteins (Nicolau et al., Proc. Natl. Acad. Sci. U.S.A. BD: 1068, 1983); and DNA coupled to a polylysine-glycoprotein carrier complex. In addition, “gene guns” have been used for gene delivery into cells (Australian Patent No. 9068389). Naked DNA, or DNA associated with liposomes, can be formulated in liquid carrier solutions for injection into interstitial spaces for transfer of DNA into cells (Feigner, WO90/11092).

Polynucleotide sequences encoding a TGF-α mutant or functional peptide fragment or mimetic, can be cloned into vectors suitable for delivery to host cells for expression. In particular retroviral vectors containing the polypeptides of the invention are particularly suitable for delivering polynucleotides to cells for gene therapy. Current strategies for gene therapy are reviewed in “The Development of Human Gene Therapy,” Ed. Theodore Friedmann, Cold Spring Harbor Laboratory Press, New York, 1999, the disclosure of which is incorporated herein.

Delivery of a polynucleotide of interest may be accomplished in vivo by administration of the vectors to an individual subject, typically by systemic administration (e.g., intravenous, intraperitoneal, intramuscular, subdermal, or intracranial infusion), Alternatively, the vectors may be used to deliver polynucleotides to cells ex vivo such as cells explanted from an individual patient (e.g., tumor-infiltrating lymphocytes, bone marrow aspirates, tissue biopsy) or universal donor hematopoietic stem cells, followed by reimplantation of the cells into a patient, usually after selection for cells which have incorporated the polynucleotide.

Vectors comprising a trophic factor-encoding nucleic acid can be introduced into a variety of cells and tissues including myeloid cells, bone marrow cells, lymphocytes, hepatocytes, fibroblasts, lung cells, epithelial cells and muscle cells. For example, polynucleotides encoding a TGF-α polypeptide may be transferred to (introduced into) stem cells.

In another aspect, a trophic factor can delivered via a cell that has been genetically modified to express the trophic factor. Thus, the present invention also encompasses gene therapy methods wherein cells are used to introduce a TGF-α mutant into a subject such that the TGF-α mutant is expressed. Such gene therapy methods may be used to treat and/or prevent conditions associated with neural degeneration. For example, a vector can be used to transfer a genetic element encoding a trophic factor, such as a TGF-α mutant, into a cell such that the cell is modified to express a TGF-α polypeptide beneficial to the treatment of a pathological disorder.

A “vector” refers to a recombinant DNA or RNA construct, such as a plasmid, a phage, recombinant virus, or other vector that, upon introduction into an appropriate host cell of the invention, results in a modification of a pre-mesenchymal, pre hematopoietic stem cell. Appropriate expression vectors are well known to those with ordinary skill in the art and include those that are replicable in eukaryotic and/or prokaryotic cells and those that remain episomal or those that integrate into the host cell genome.

Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing gene expression and function are known to those skilled in the art. Gene presence, amplification, and/or expression may be measured in a sample directly, for example, by conventional Southern blotting, Northern blotting to quantitate the transcription of mRNA, dot blotting (DNA or RNA analysis), or in situ hybridization, using an appropriately labeled probe which may be based on a sequence provided herein. Those skilled in the art will readily envisage how these methods may be modified, if desired.

The invention provides a method of modulating progenitor cell proliferation, and/or migration, and/or differentiation by introducing an effective amount of a trophic factor into a subject such that progenitor cells are induced to proliferate, and/or migrate and/or differentiate at or into the site of neurological damage or injury. Peripheral administration of a TGF-α mutant provides an effective method for the treatment of a number of diseases and disorders associated neurological deficits.

The instant methods can be used, for example, to treat any condition affected by impairments of the dopaminergic pathway causing neurologic deficits and/or neuropsychiatric disorders. Neurological deficits that can be treated using the instant methods include, but are not limited to, degenerative diseases, such as Alzheimer's Disease (AD), Parkinson's Disease (PD), Amyotrophic lateral sclerosis (ALS), Huntington's Disease (HD), Pick's disease, progressive supranuclear palsy, striatonigral degeneration, cortico-basal degeneration, olivopontocerebellar atrophy, Leigh's disease, infantile necrotizing encephalomyelopathy, Hunter's disease, mucopolysaccharidosis, various leukodystrophies (such as Krabbe's disease, Pelizaeus-Merzbacher disease and the like), amaurotic (familial) idiocy, Kufs disease, Spielmayer-Vogt disease, Tay Sachs disease, Batten disease, Jansky-Bielschowsky disease, Reye's disease, cerebral ataxia, chronic alcoholism, beriberi, Hallervorden-Spatz; syndrome, cerebellar degeneration, and the like. In addition, the methods disclosed herein can be used, for example, to improve mood, reduce anxiety, treat depression, improve cognition, improve memory and improve learning.

For example, dopaminergic cells are known to be lost in association with a number of debilitating neurodegenerative diseases such as Parkinson's disease. In the cerebral cortex, neurons and glia are lost following ischemic episodes caused by a thrombus or embolus, in the spinal cord, motor neurons are particularly susceptible to damage due to a traumatic injury. The tissue may be disrupted by physical force (e.g., ablating or excising neurons, or severing one or more of the processes that extend from the neuronal cell bodies) or by applying a chemical substance such as a toxin or neurotoxin (e.g., ricin or 6-OHDA), a corrosive chemical (e.g., an acidic or basic solution), a compound that induces apoptosis (see, e.g., Leavitt et al., Soc. Neurosci. Abstr. 22:505, 1996), a compound that induces demyelination (see, e.g., Lachapelle et al., Soc. Neurosci. Abstr. 23:1689, 1997), or a compound capable of inhibiting the activity of the cell, e.g., an antisense oligonucleotide (such as an oligonucleotide that inhibits transcription of the gene encoding the cell's primary neurotransmitter), an antibody, or a polypeptide. Many such compounds are known to those of ordinary skill in the art and include compounds that bind to, but fail to activate, a receptor on the cell surface, such as the metabotropic receptors normally bound by glutamate. Moreover, neural tissue may be disrupted by the administration of chemotherapeutic agents, such as radiation or chemical compositions, necessary to treat a neoplastic lesion of the CNS. Thus, the invention encompasses the peripheral administration of a TGF-α mutant prior to, during or subsequent to the administration of a chemotherapeutic agent to the CNS for the purpose of regenerating neural tissue damaged by a necessary medical treatment. The medical treatment can include, for example, surgical procedures that disrupt tissue of the CNS.

Accordingly, in one embodiment, a TGF-α mutant polypeptide, fragment or mimetic can be used to treat, repair or regenerate a tissue or a subject having a neurological injury. In one embodiment, the invention provides a continuous or intermittent infusion of a TGF-α mutant polypeptide, a related polypeptide, mimetic, or functional fragment thereof having TGF-α mutant activity peripheral to a site of injury or at a site that allows for delivery of the TGF-α mutant polypeptide, fragment or mimetic to the site of injury (e.g., a vein or portal upstream of the injured site). The TGF-α mutant polypeptide, a related polypeptide, mimetic, or functional fragment thereof having TGF-α mutant activity of the invention delivered to the site of injury promotes the proliferation, and/or migration, and/or differentiation of progenitor cells that are located at the site of injury, or circulating in the lumen of a blood vessel near the site of injury, or are located in the wall of the blood vessel near the site of injury, and thus promotes tissue repair and regeneration.

In another embodiment, administration of a TGF-α mutant polypeptide, a related polypeptide, mimetic, or functional fragment thereof having TGF-α mutant activity to a subject peripheral to a site of injury or at a site that allows for delivery of the TGF-α mutant polypeptide, fragment or mimetic to the site of injury (e.g., a vein or portal upstream of the injured site) and to the location of a progenitor cell capable of differentiating into a neuronal cell. For example, the invention provides a mechanism fur inducing an endogenous progenitor cell to migrate and/or differentiate into a neuronal cell at a site of CNS damage. The progenitor cell can be, for example, a hematopoietic progenitor cell which is located at or near the site of CNS damage or is located distally from the site of damage. Such cells generally reside in bone marrow tissue, but can be circulating throughout the subject as well as stationary in a blood vessel at or near the site of injury or damage. Thus, the TGF-α mutant polypeptide, a related polypeptide, TGF-α mimetic, or functional fragment thereof having TGF-α mutant activity of the invention delivered to the site of injury promotes the proliferation, migration, and/or differentiation of progenitor cells that are located at the site of injury, or circulating in the lumen of a blood vessel near the site of injury, or are located in the wall of the blood vessel near the site of injury, and thus promotes tissue repair and regeneration.

Further, injuries (traumatic or neurotoxic) that cause a loss of neuronal function can be treated by the functional polypeptides and mimetics of the invention.

Reid et al, describes the continuous infusion of TGF-α into brain tissue Mowing injury (see, WO 99/06060, and U.S. patent application Ser. No. 09/129,028 which are incorporated herein by reference in their entirety). The present invention describes methods whereby peripheral administration of a TGF-α mutant polypeptide, a related polypeptide, mimetic, or a fragment having TGF-α mutant activity, either before, prior to, simultaneous with or following tissue injury can stimulate progenitor cells at or near the damaged tissue or distal from the damaged or injured tissue to proliferate, migrate and differentiate to replace or repair cells at the site of injury and to ameliorate the effects of such damage.

The term “ameliorate” denotes a lessening of the detrimental effect of the disease-inducing response in the patient receiving therapy. The terms “treating,” “treatment,” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

Generally, the terms “treating”, “treatment” and the like are used herein to mean affecting a subject, tissue or cell to obtain a desired pharmacologic and/or physiologic effect. The effect may be prophylactic in terms of completely or partially preventing a. disease or disorder or sign or symptom thereof, and/or may be therapeutic in terms of a partial or complete cure for a disorder or disease and/or adverse effect attributable to the disorder or disease. “Treating” as used herein covers any treatment of, or prevention of, or inhibition of a disorder or disease in a subject, The subject can be an invertebrate, a vertebrate, a mammal, and particularly a human, and includes by way of example: (a) preventing the disease or disorder from occurring in a subject that may be predisposed to the disease or disorder, but has not yet been diagnosed as having it; (b) inhibiting the disease or disorder, i.e., arresting its progression; or (c) relieving or ameliorating the disease or disorder, i.e., causing regression. Thus, treating as used herein includes, for example, repair and regeneration of damaged or injured tissue or cells at the site of injury or prophylactic treatments to prevent damage, e.g., before chemotherapy.

The present invention provides for peripheral administration of TGF-α mutant polypeptide, a related polypeptide, a mimetic, or a functional fragment thereof having the TGF-α mutant activity. Peripheral administration of TGFα mutant can be as a single dose, or multiple doses, or continuous (e.g., continuous infusion). Multiple doses can be administered hourly, daily, weekly, or monthly, depending, in part, on the severity of the injury or damage, the response of the individual to treatment, etc.

A single, multiple, or continuous dose peripheral injection can be administered within minutes (e.g., within about 5 to about 120 minutes), within hours (e.g., within about 2 hours to about 36 hours), within days (e.g., within about 2 days to about 14 days), or within weeks (e.g. within about 2 weeks to about 8 weeks) after tissue damage or injury.

The administration of these compounds induces modulation of neural activity or neurogenesis, i.e., the administration modulates neurogenesis or neural activity. As used herein the term “modulating” or “modulation” including any change in neural activity or any neurogenesis, which includes, but is not limited to, differentiation (including redifferentiation or dedifferentiation), migration, and proliferation of neurons, neural stem cells or any other cell, including progenitor cells or stem cells.

In one aspect, the initial events in the modulation of neural activity or neurogenesis including migration and/or differentiation of cells to the CNS from an endothelium or from a perivascular cell in the CNS. Alternatively, the initial events in the modulation of neural activity or neurogenesis including migration and/or differentiation of cells to the CNS from an non-CNS tissue compartment, such as the bone marrow, the blood, the spleen, a lymph node, and the like, Modulation of neural activity or neurogenesis can also include migration and/or differentiation of cells at a site of injury or damage in the CNS of a subject. The modulation includes, but is not limited to, progenitor cell (or progeny thereof) proliferation, migration, differentiation (including dedifferentiation or redifferentiation) of cells, including endogenous progenitor cells. Such cells can be located distally from the site of injury, such as in a vascular endothelium, perivascularly, or recruited to the CNS for any tissue or cell reservoir, such as the bone marrow or spleen. Alternatively, such cell can be located at or near the site of tissue injury.

Compositions (e.g., as a single unit dosage delivery) also can be administered immediately adjacent to the site of injury or can be, for example, to a vessel (blood or lymphatic) that drains or flows to the site of injury.

Methods of administration include, but are not limited to, subcutaneous, topical, oral, intradermal, intramuscular, intraperitoneal, intravascular (e.g., intravenous), subcutaneous, intranasal, and epidural routes. The cells of the invention may be administered by any convenient route, for example, by infusion or bolus injection, by absorption through epithelial or mucocutaneous linings (e.g., oral mucosa, rectal and intestinal mucosa, etc.), and may be administered together with other biologically active agents. Administration can be systemic or local.

In alternative aspects, the trophic factor is peripherally administered to a subject in need of such treatment by, for example and not by way of limitation, infusion during surgery, injection, a catheter means, or an implant means, the implant being of a porous, non-porous, or gelatinous material, including membranes, such as sialastic membranes, or fibers. Alternatively, a trophic factor can be included in a controlled release matrix which can be positioned in proximity to, but not in direct contact with, damaged neural tissue thereby promoting regeneration of such tissue. The term “controlled release matrix” means any composition that allows the slow release of a trophic factor which is mixed or admixed therein. The matrix can be a solid composition, a porous material, or a semi-solid, gel or liquid suspension containing bioactive substances.

Injuries treatable by the methods of the invention include, tier example, gunshot wounds, injuries caused by blunt force, penetration injuries, injuries caused by surgical procedure (e.g., tumor removal, abscess removal, epilepsy lesion removal) poisoning (e.g., carbon monoxide), shaken baby syndrome, adverse reactions to medications, drug overdoses, and post-traumatic encephalopathy. Ischemia can further cause CNS injury due to disruption of blood flow or oxygen delivery that can kill or injure neurons and glial cells. Such injuries can be treated by administration of the TGF-α mutant polypeptides and include, for example, injuries caused by stroke, anoxia, hypoxia, partial drowning, myoclomis, severe smoke inhalation, dystonias, and acquired hydrocephalus. Developmental disorders that can be treated by the functional peptides include, for example, schizophrenia, certain forms of severe mental retardation, cerebral palsey, congenital hydrocephalus, severe autism, Downs Syndrome, LHRH/hypothalamic disorder, and spina bifida. The method can be further used to treat disorders affecting vision caused by the loss or failure of retinal cells and include, for example, diabetic retinopathy, serious retinal detachment (associated with glaucoma), traumatic injury to the retina, retinal vascular occlusion, macular degeneration, optic nerve atrophy and other retinal degenerative diseases. For example, Young et al. (Mol. Cell. Neurosciences 16:197, 2000) have found that adult neural stem cells may be useful in treating blindness due to degradation of the retinas.

Injuries to the spinal cord and associated ganglia can be treated by the methods of the invention. Examples of spinal cord injuries are post-polio syndrome, amyotrophic lateral sclerosis (ALS), traumatic injury, surgical injury, and paralytic diseases. Demyelinating autoimmune disorders can be treated by administration of the functional peptides and include, for example, multiple sclerosis. The functional peptides can also be used to treat neurological deficits caused by infection of inflammatory diseases, including, for example, Creutzfeldt-Jacob disease and other slow virus infectious diseases of the CNS, AIDS encephalopathy, post-encephalitic Parkinsonism, viral encephalitis, bacterial meningitis and other CNS effects of infectious diseases.

The invention includes various pharmaceutical compositions useful for delivery or administration of a polypeptide, peptide or mimetic useful in the method of the invention. In one embodiment, the pharmaceutical compositions are useful in managing or treating neuronal tissue damage and cell renewal in a subject. The pharmaceutical compositions according to the invention are prepared by bringing a polypeptide or peptide derivative of a TGF-α mutant into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases.

Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary, XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

In some embodiments, pharmaceutical compositions are prepared and administered in dose units. Solid dose units are tablets, capsules and suppositories and including, for example, alginate based pH dependent release gel caps. For treatment of a subject, depending on activity of the compound, manner of administration, nature and severity of the disorder, age and body weight of the subject, different daily doses are necessary. Under certain circumstances, however, higher or lower daily doses may be appropriate. The administration of the daily dose can be carried out both by single administration in the form of an individual dose unit or by several smaller dose units and also by multiple administrations of subdivided doses at specific intervals.

The pharmaceutical compositions according to the invention may be administered systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al (eds.) (1990), each of which is herein incorporated by reference. As used herein, a “therapeutically effective” amount of a composition containing a trophic factor that promotes neural regeneration is an amount that is effective in inducing the proliferation, migration, or differentiation of a progenitor cell, or progeny thereof, at or into a site of injury or damage in the central nervous system of the subject, subsequent to peripheral administration of the trophic factor. The term “induce” or “induction” as used herein, refers to the activation, stimulation, enhancement, initiation and or maintenance of the cellular mechanisms or processes necessary for the formation of any of the tissue, repair process or development as described herein.

“Administering” the pharmaceutical composition of the invention may be accomplished by any means known to the skilled artisan. A “subject” refers to a mammal, e.g., a human. The TGF-α mutant polypeptide or functional fragment can be administered parenterally, enterically, by injection, rapid infusion, nasopharyngeal absorption, dermal absorption, rectally and orally. Pharmaceutically acceptable carrier preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate.

Carriers for occlusive dressings can be used to increase skin permeability and enhance absorption. Liquid dosage forms for oral administration may generally comprise a liposome solution containing the liquid dosage form. Suitable solid or liquid pharmaceutical preparation forms are, for example, granules, powders, tablets, coated. tablets, microcapsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners and elixirs containing inert diluents commonly used in the art, such as purified water.

Sterile injectable solutions can be prepared by incorporating the active agent (e.g., TGF-α mutant protein) in the required amount (e.g., about 10 μg to about 10 mg/kg) in an appropriate solvent and then sterilizing, such as by sterile filtration. Further, powders can be prepared by standard techniques such as freeze-drying or vacuum drying.

In another embodiment, the active agent is prepared with a biodegradable carrier for sustained release characteristics for either sustained release in the subject or target organ by implantation with long-term active agent release characteristics to the intended site of activity. Biodegradable polymers include, for example, ethylene vinyl acetate, polyanhydrides, polyglycolic acids, polylactic acids, collagen, polyorthoesters, and poly acetic acid. Liposomal formulation can also be used.

Kits

The invention provides kits that contain the pharmaceutical compositions comprising a trophic factor, for use in carrying out the instant methods. The kit can contain instructional material teaching methodologies, e.g., methods for peripherally administering a TGF-α mutant polypeptide (or a related polypeptide, a functional fragment, or a mimetic thereof) formulation. Kits containing pharmaceutical preparations can include directions as to indications, dosages, routes and methods of administration, and the like.

It must be noted that as used herein and in the appended claims, the singular forms “a,” “and,” and “the” include plural referents, unless the context clearly dictates otherwise. Thus, for example, reference to “a target cell” includes a plurality of such cell, and reference to “the expression vector” includes reference to one or more transformation vectors and equivalents thereof known to those skilled in the art, and so forth.

Unless defined otherwise, all technical and scientific terms used herein have the same meaning as commonly understood to one of ordinary skill in the art to which this invention belongs. Any methods, cells and genes similar or equivalent to those described herein can be used in the practice or testing of the invention.

Without further elaboration, it is believed that one skilled in the art can, using the preceding description, utilize the present invention to its fullest extent. The following examples are to be considered illustrative and thus are not limiting of the remainder of the disclosure in any way whatsoever.

### EXAMPLES

Materials: The following materials were used in the examples below.

Human TGFα expression clone (Invivogen, #pORF9-hTGFA)

Human TGFα, 200 ug (Sino Biological, #11252-HNAE)

Human EGFR ECD, 100 ug (Sino Biological. #10001-H02H)

Human TGFα biotinylated antibody, 50 ug (R&D System, #BAF239)

Human TGFα antibody,100 ug (R&D System, #AF-239-NA)

PathScan total EGF receptor sandwich ELISA kit (Cell Signaling, #7250S)

PathScan Phospho-EGF receptor (tyr 1068) sandwich ELISA kit (Cell Signaling, #7240S)

### Example 1

### Cloning and Expression of hTGFα and Assay Implementation Assay implementation

Quantitation sandwich ELISA was set up to measure the concentration of TGFα. Affinity ELISA, cell-based ELISA, FACS analysis and cell-based EGFR phosphorylation assays were set up to determine the activity of TGFα.

1. Establishment of Quantitation ELISA to Determine the Expression of Human TGFα

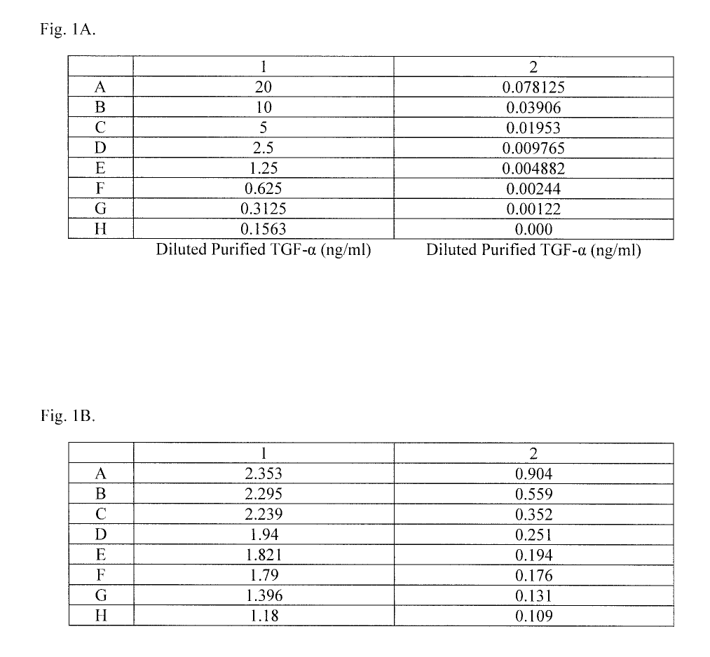

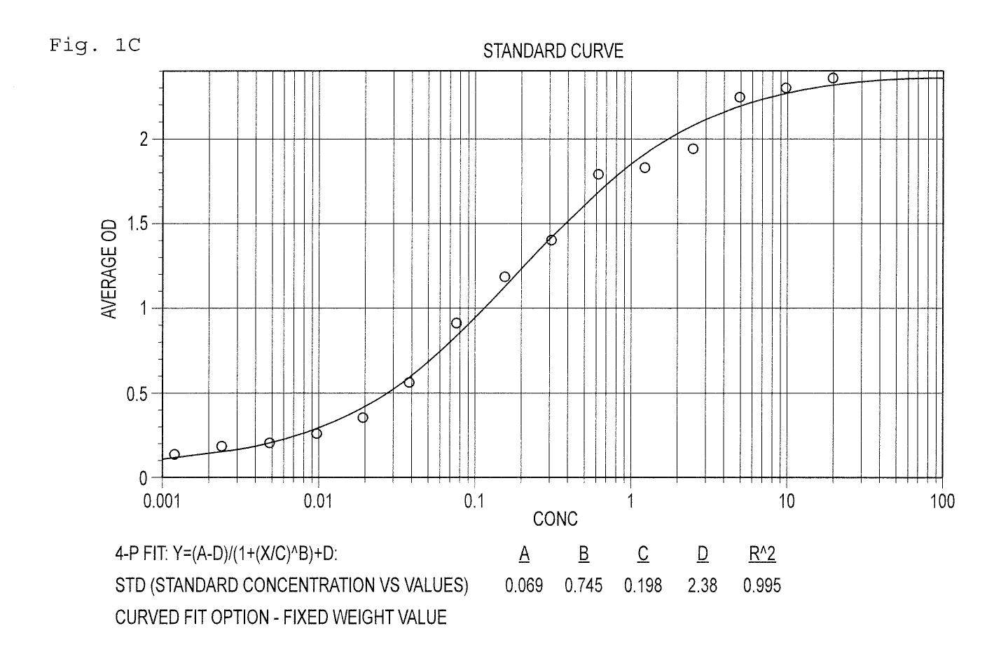

A Quantitation sandwich ELISA protocol was established to determine the concentration of human TGFα (hTGFα). The wells of a 96-well plate were coated with anti-hTGFα (R&D Systems, #AF-239-NA). Two fold serial dilutions of a 20 ng/ml solution of human TGFα (hTGFα) were made, aliquots from each sample added to the wells (FIG. 1A) and the plates incubated to allow binding. Bound hTGFα was detected using biotinylated anti-hTGFα (R&D Systems #BAF239) and Streptavidin conjugated with HRP. TMB substrate was added to the wells, the reactions stopped after 1 minute, and the OD450nm value of the samples measured using a Molecular Device SPECTRAmax Plus. (FIG. 1B). Softmax was then used to produce a standard curve based on the OD450 nm values (FIG. 1C). The results showed that the quantitative sandwich ELISA can be used to determine the concentration of hTGFα present in the bacteria lysate or in the CHO transfection supernatant.

2. Establishment of Affinity ELISA, Cell Based ELISA, FACS Analysis and Cell Based EGF Phosphorylation Assay to Measure the Activity of hTGFα

a. Affinity ELISA

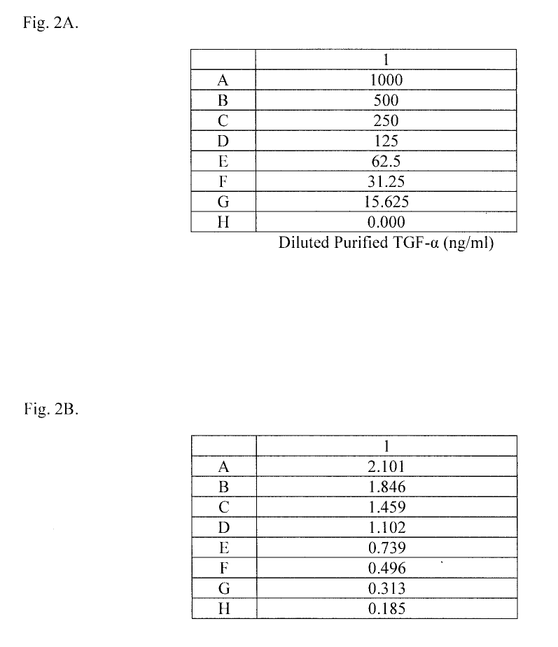

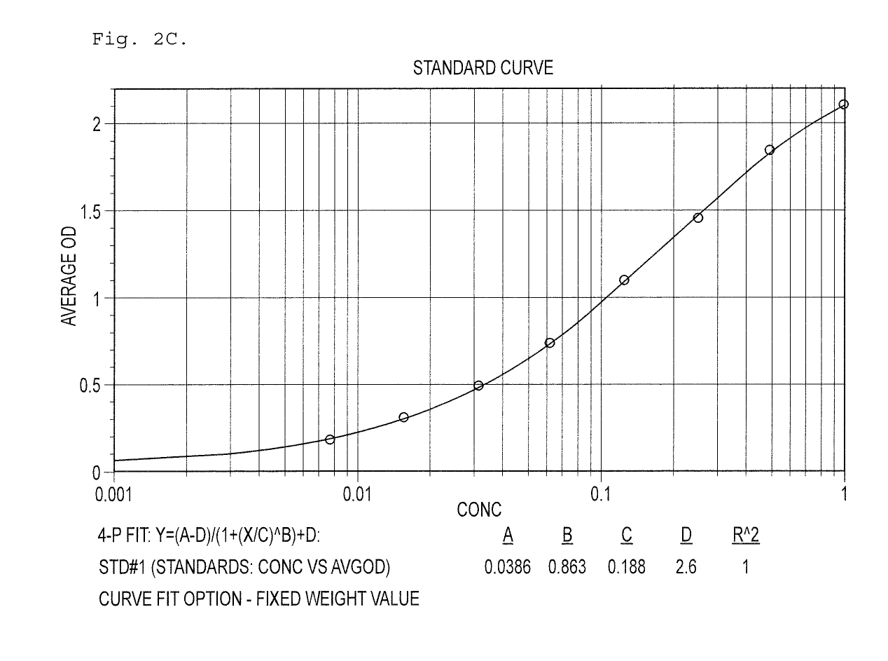

An affinity ELISA protocol was developed to determine the binding activity of hTGFα to EGFR. Briefly, EGFR extra cellular domain (EGFR ECD) (Sinobiological, #10001-H020H) was immobilized in the wells of a 96-well plate. Two fold serial dilutions of a solution of 1000 ng/ml human TGFα (hTGFα) were made, aliquots of each sample added to the wells, and the plate incubated to allow binding to occur. (FIG. 2A). Bound TGFα was detected using a biotinylated anti-hTGFα antibody (R&D Systems #BAF239) and Streptavidin conjugated with HRP. TMB substrate was added to the wells, the reactions stopped after 1 minute, and the OD450nm value of the samples measured using a Molecular Device SPECTRAmax Plus (FIG. 2B). Softmax was then used to produce a standard curve based on the OD450 nm values (FIG. 2C). The results showed that the dynamic range of the affinity ELISA is sufficient to measure the improved affinity of the CPE™ and CPS™ mutants compared to the wild type either in the bacterial lysate or in the CHO transfection supernatant.

b. Cell Based ELISA and FACS Analysis

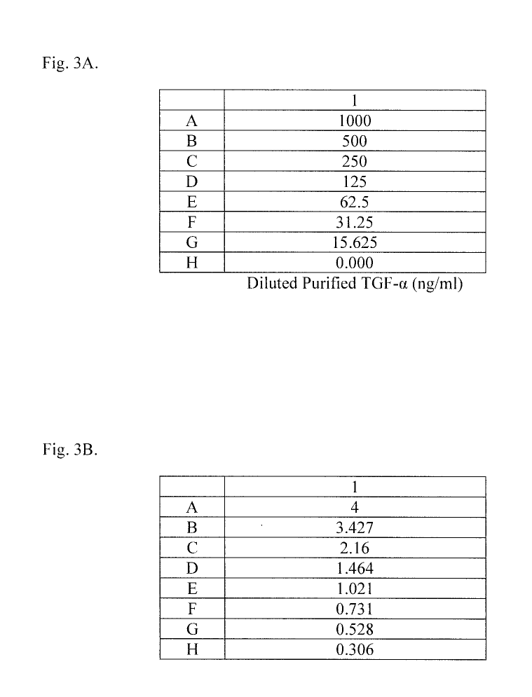

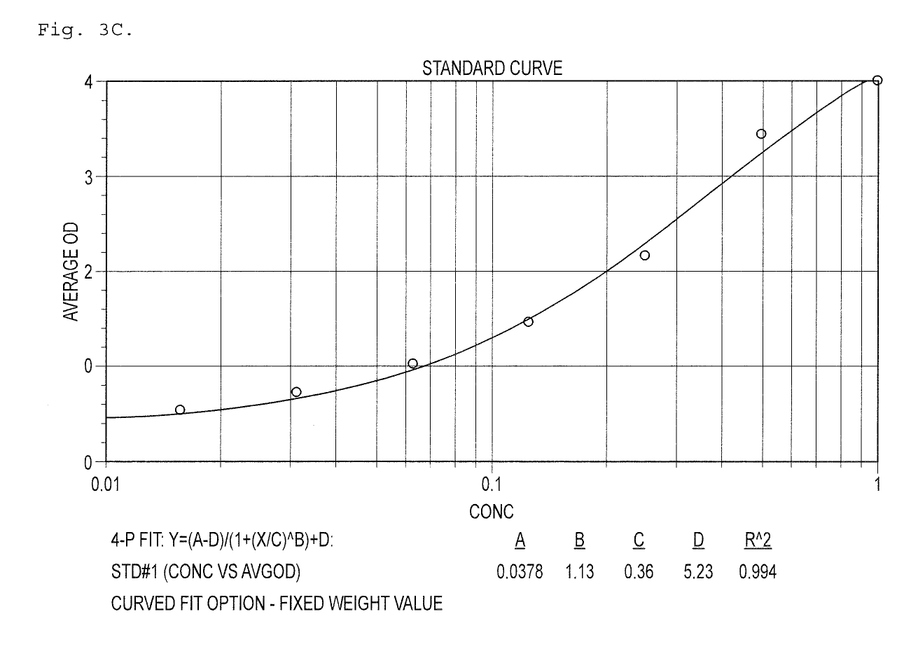

Binding activity of hTGFα to EGFR was also confirmed by cell based ELISA and FACS analysis using A431 cells, which are known to express high levels of EGFR on their surface. For the cell-based assay, A431 cells (ATCC#CRL1555) were seeded into the wells of a 96 well plate. A 1000 ng/ml solution of human TGFα (hTGFα) was serially diluted (two-fold dilutions), aliquots of each dilution added to the immobilized cells, and the plates incubated to allow binding. (FIG. 3A) After washing, bound hTGFα was detected using biotinylated anti-hTGFα (R&D Systems #BAF239) and Streptavidin conjugated with HRP. TMB was added to each well, the reactions stopped after four minutes, and the samples read as described above in in paragraph a). (FIG. 3B) The standard curve was generated using Softmax. (FIG. 3C)

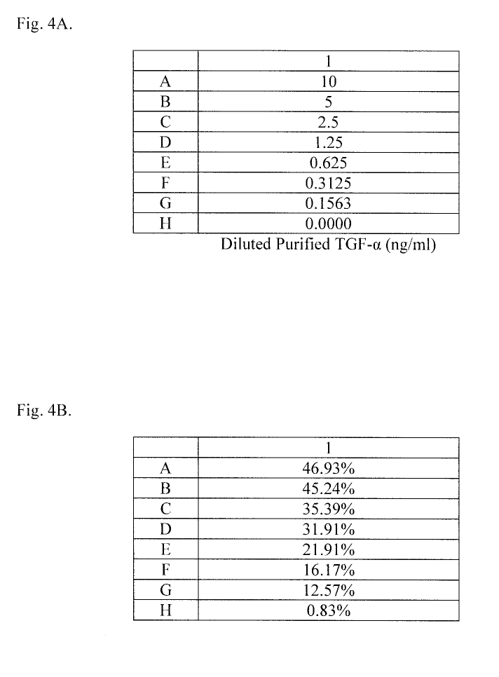

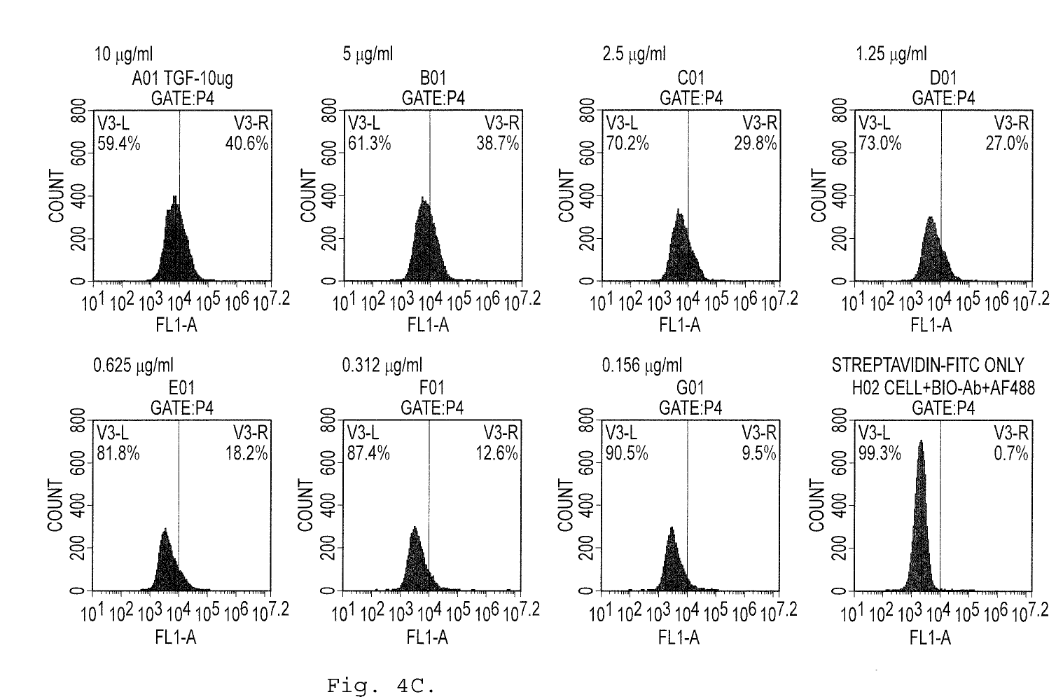

For the FACS analysis, two fold serial dilutions of a 10 ug/ml solution of hTGFα were made, and aliquots of each dilution incubated with A431 cells (ATCC#CRL1555) (FIG. 4A). Bound hTGFα was detected using biotinylated anti-hTGFα (R&D Systems, #BAF239) and Streptavidin conjugated with FITC. Cells were analyzed with a BD Accuri C6 FACS analyzer to determine the percent of cells bound by hTGFα . (FIG. 4B and FIG. 4C) The results showed that the dynamic range of the cell based ELISA is sufficient to measure the improved affinity of the CPE™ and CPS™ mutants compared to the wild type expressed either in the bacterial lysate or in the CHO transfection supernatant. The results also show that the cell based ELISA can be used for high through screening of the CPE™ and CPS™ mutants. The dynamic range of the FACS analysis is not as wide as the dynamic range of affinity ELISA or cell based ELISA but it can be used to confirm the binding activities of the mutants with improved binding activities.

c. Cell Based EGFR Phosphorylation Assay

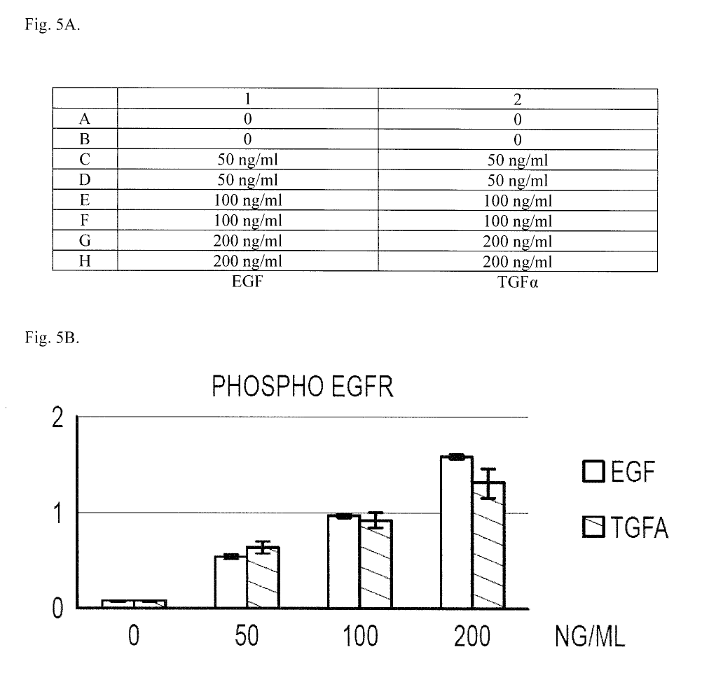

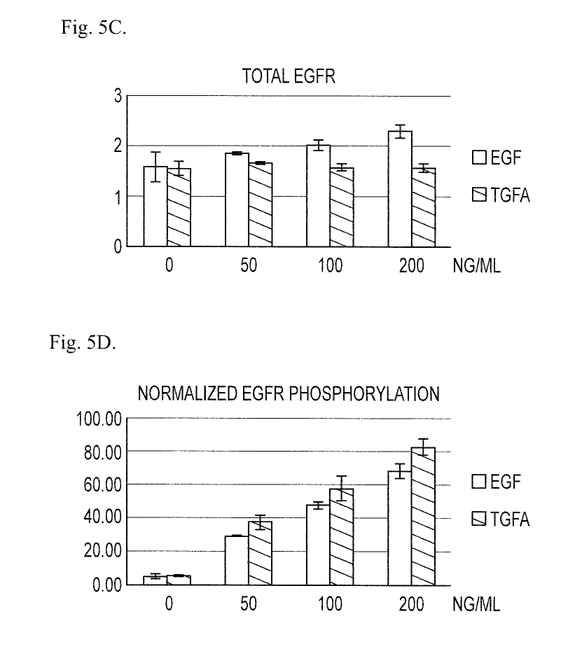

A cell based EGFR phosphorylation assay was set up to determine the binding activity of hTGFα to EGFR using a functional assay. A431 cells were seeded in 96 well formats and were serum starved overnight. The cells were washed in serum free media and incubated with 50 ul of 0, 50, 100 or 200 ng/ml of EGF (FIG. 5A, column 1) or hTGFα (FIG. 5A, column 2) in DMEM at 37° C. for 10 min: The cells were then washed with PBS and lysed. Half of the cell lysate were used to determine the level of total EGFR (FIG. 5C) using Cell Signaling kit #7250 and the other half of the cell lysate were used to determined the level of phosphorylated EGFR (FIG. 5B) using Cell Signaling kit #7240 following vendor's protocols. The results showed that the cell based EGFR phosphorylation assay can be used to confirm the ability of CPE™ and CPS™ mutants to bind and activate EGFR on the cell surface.

Cloning/expression of hTGFα: hTGFα was cloned into BioAtla's bacterial and mammalian expression vectors. The hTGFα bacterial expression constructs were expressed in E. coli BL21 (DE3) cells, and the expression level determined using both SDS/PAGE and quantitation ELISA.

The hTGFα mammalian expression constructs were expressed in CHO cells and the expression level of hTGFα was determined using quantitation ELISA. Activity of hTGFα expressed in bacteria and in CHO cells was confirmed using cell-based ELISA, FACS analysis and cell-based EGFR phosphorylation assay described above.

1. Construction of hTGFα Expression Plasmids

hTGFα mammalian expression and bacterial expression plasmids were constructed. Both pro and mature hTGFα (SEQ ID NO:148 and SEQ ID NO:146) were used as templates for cloning into mammalian expression plasmid (BAP060-hTGFα -M, BAP060-mhTGFα-M) and mature hTGFα (SEQ ID NO:144) as a template for cloning into bacterial periplasmic expression plasmid (BAP060-hTGFα-B). The hTGFα expression constructs were verified by sequencing and are listed in Table 1.

TABLE 1
BAP060 Expression Constructs

| | | Expression | |
|---|---|---|---|
| Clone ID | Description | host | SEQ ID NO: |
| BAP060- | Mature | E-coli | Contains SEQ ID |
| hTGFα-B | hTGFα | periplasmic | N0: 144 encoding |
| | (AA 40-89) | | SEQ ID NO: 145 |
| | with PelB | | |
| | signal sequence | | |
| BAP060- | Full length | Mammalian - | Contains SEQ ID |
| hTGFα-M | hTGFα | secreted | NO: 148 encoding |
| | (AA 1-89) | | SEQ ID NO: 149 |
| BAP060- | Full length | Mammalian - | Contains SEQ ID |
| mhTGFα-M | hTGFα | secreted | N0: 146 encoding |
| | (AA 40-89) | | SEQ ID NO: 147 |
| | with BioAtla | | |
| | signal sequence | | |

2. Expression of hTGFα in E. coli BL21 (DE3) cells and determination of activity

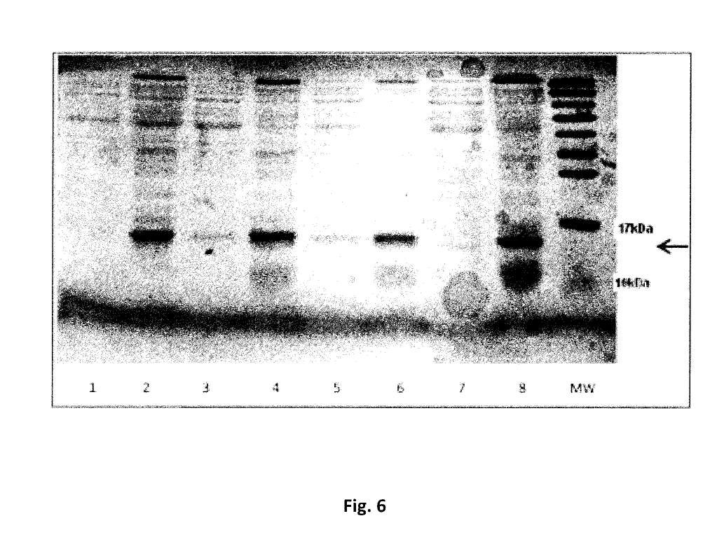

E. coli BL21 cells were transformed with BAP060-hTGFα-B and protein expression induced by addition of 1 mM isopropyl-β-D-thiogalactoside (IPTG) to each culture. The cultures were incubated at 37° C. for 3 hours (FIG. 6, lanes 3 and 4), 30° C. for 6 hours (FIG. 6, lanes 5 and 6) or 25° C. overnight (FIG. 6, lanes 7 and 8) in 14 ml tubes. Un-induced cultures were also included (lanes 1 and 2). The periplasmic and insoluble fractions were collected. The periplasmic proteins were prepared by incubating the cells in OS buffer (200 mM Tris-HCl, pH 7.5, 20% sucrose, 1 mM EDTA) with DNAse, RNAse and lysozyme. After addition of lysis buffer (10 mM Tris, pH 7.5, 50 mM KCl, 1 mM EDTA and 0.1% of sodium deoxycholate) to the cells in OS buffer, the cells were centrifuged. The supernatants (lanes 1, 3, 5, 7) which contain the periplasmic fractions were transferred to another microcentrifuge tube. The cell pellet (lanes 2, 4, 6, 8) was re-suspended in 100 μl SDS/PAGE gel loading buffer. 15 μl of supernatant and pellet were separated on 15% SDS polyacrylamide gels and visualized with Coomassie Blue. 17 kDa and 10 kDa molecular weight standards are shown. The results are shown in FIG. 6. The arrow indicates hTGFα.

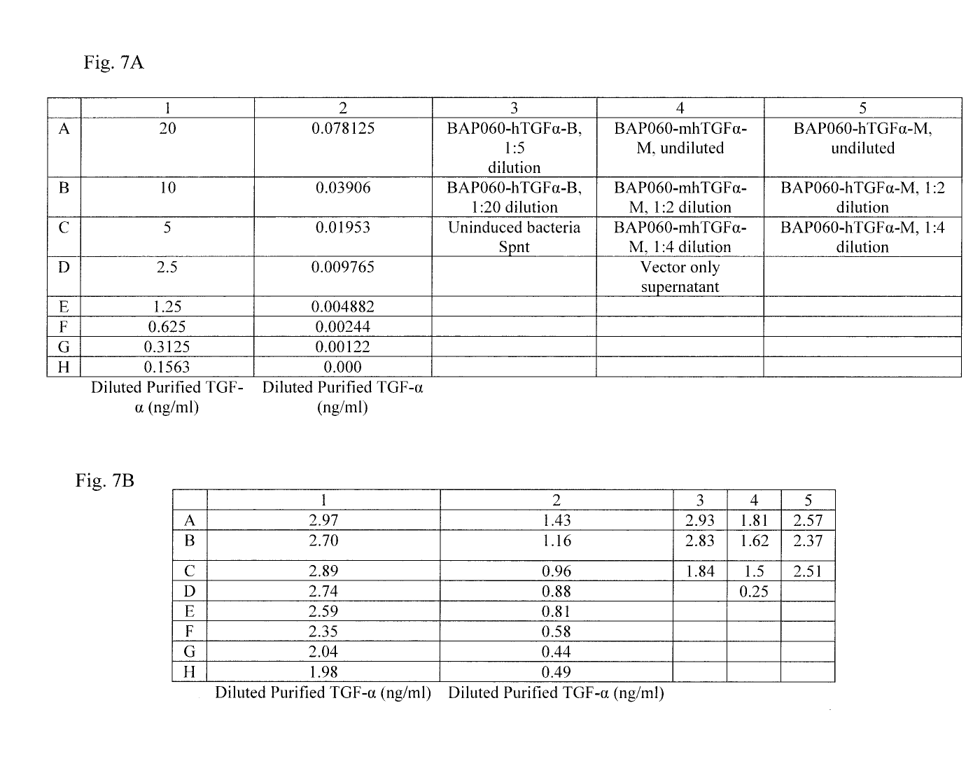

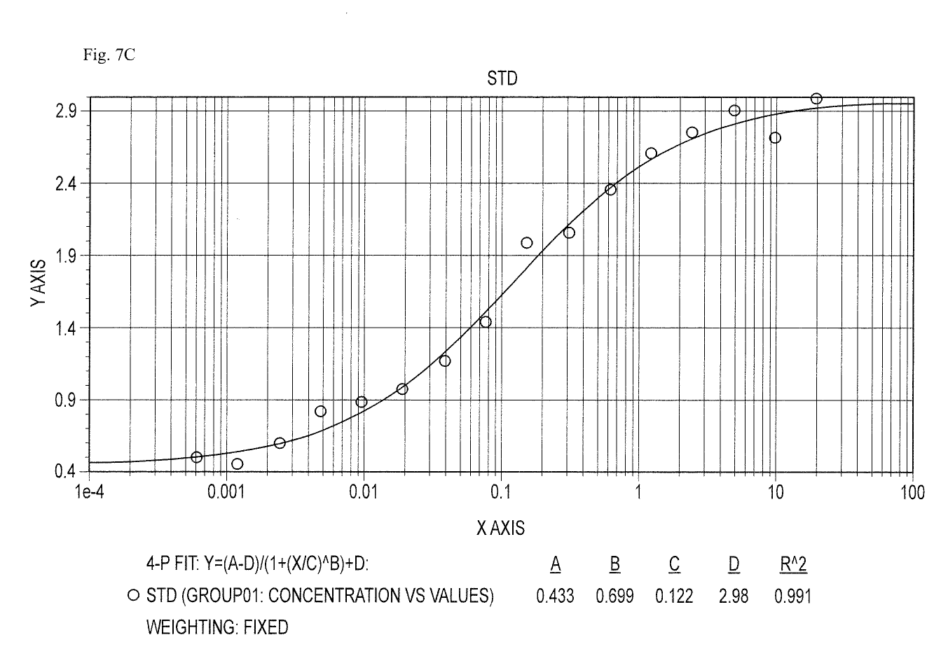

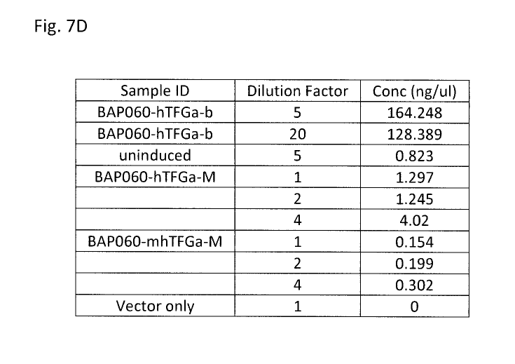

The expression levels were also measured by quantitation ELISA. Two-fold, serially diluted hTGFα standards, starting at 20 ng/ml (columns 1 and 2), 1:5 or 1:20 diluted bacterially expressed hTGFα (column3), and two-fold serially diluted mammalian expressed hTFGα (CHO cells transfected with BAP060-mhTFGα-M, column 4, or CHO cells transfected with BAP060-hTFGα-M, FIG. 7A,column 5) were incubated with anti-TFGα (R&D Systems, #AF-239-NA) immobilized on the plate. Bound TFGα was detected using biotinylated anti-TFGα (R&D Systems, #BAF239) and Streptavidin conjugated with HRP. TMB was added to each well and after three minutes, the reactions stopped and the OD450 nm values of the wells measured using a Molecular Device SPECTRAmax Plus. A standard curve was then generated using Softmax and levels of recombinant proteins determined based on the standard curve. The results are shown in FIGS. 7B-7D.

Although the majority of hTGFα was present in the insoluble pellet fractions, a percentage of hTGFα was present in the periplasmic fractions. The expression levels of hTGFα induced at all three temperatures are comparable; therefore, it was decided to use 37° C. for three hours as the induction conditions.

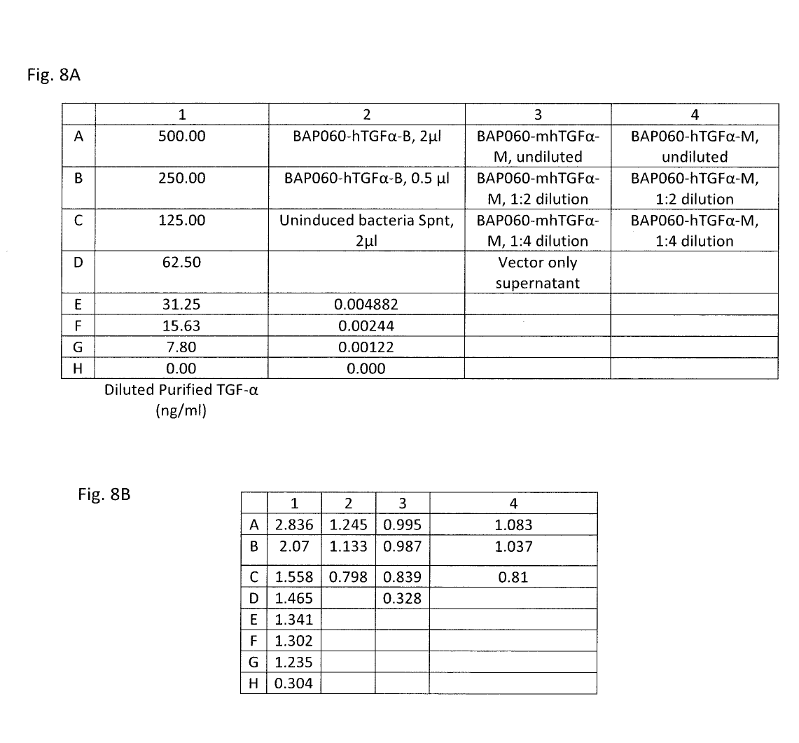

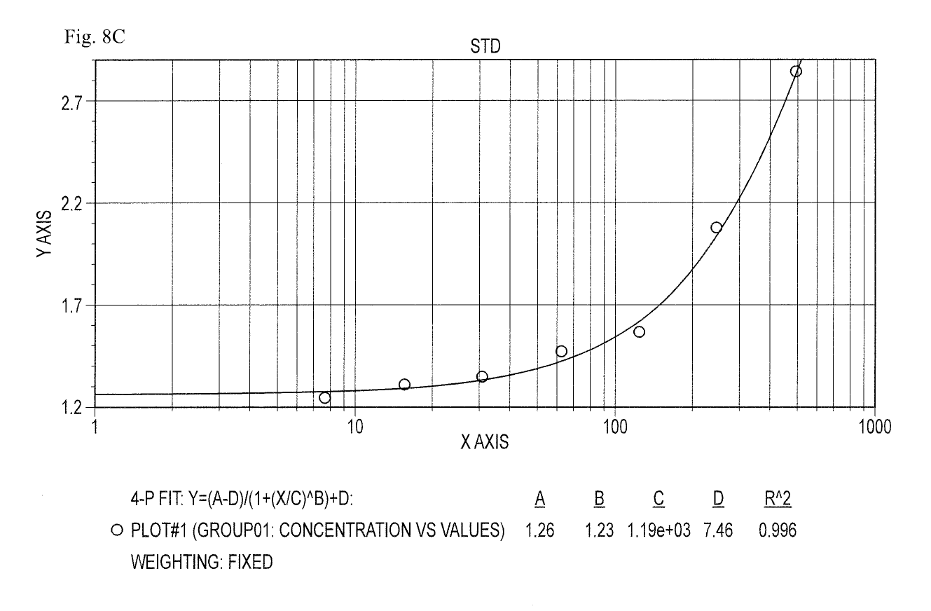

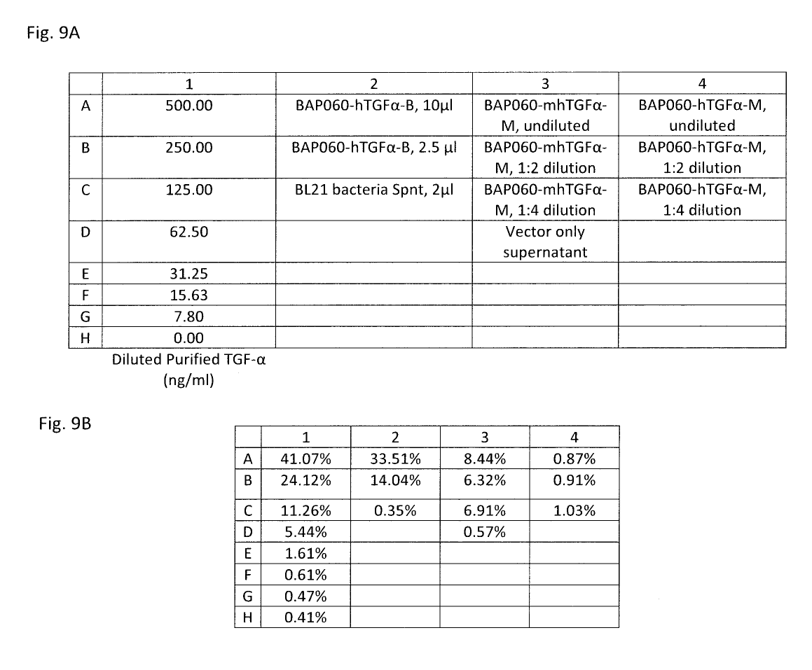

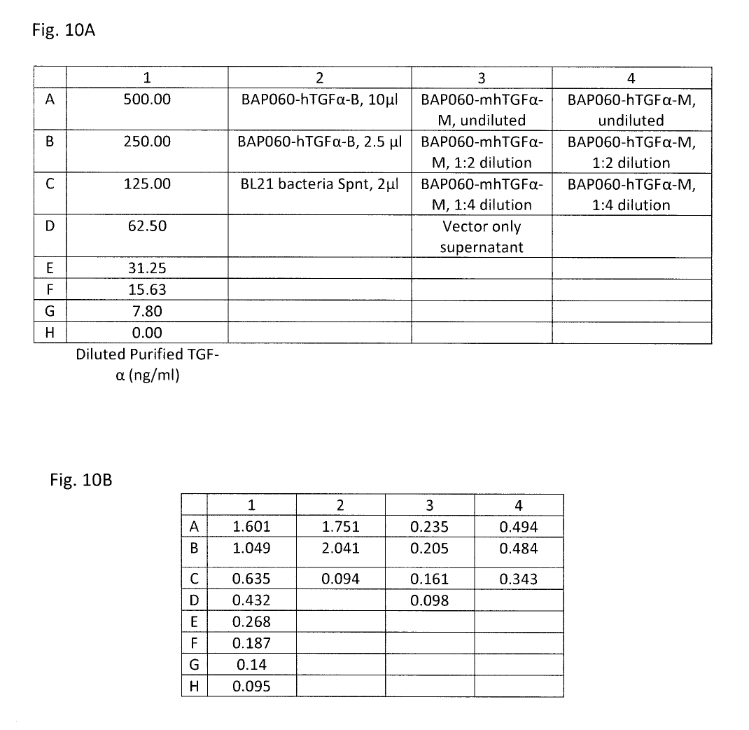

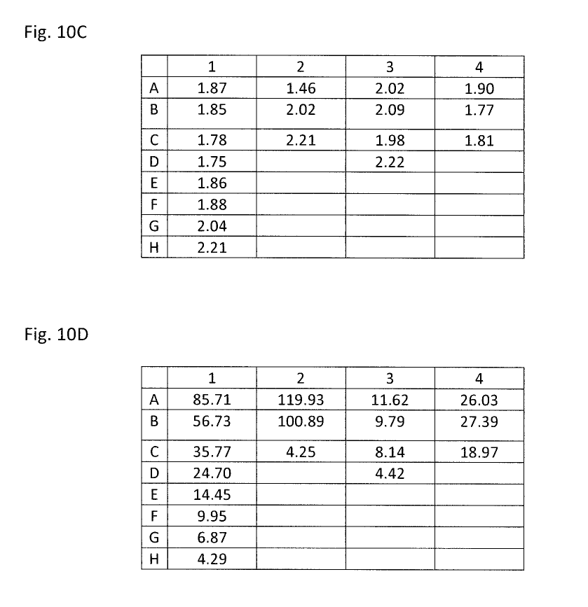

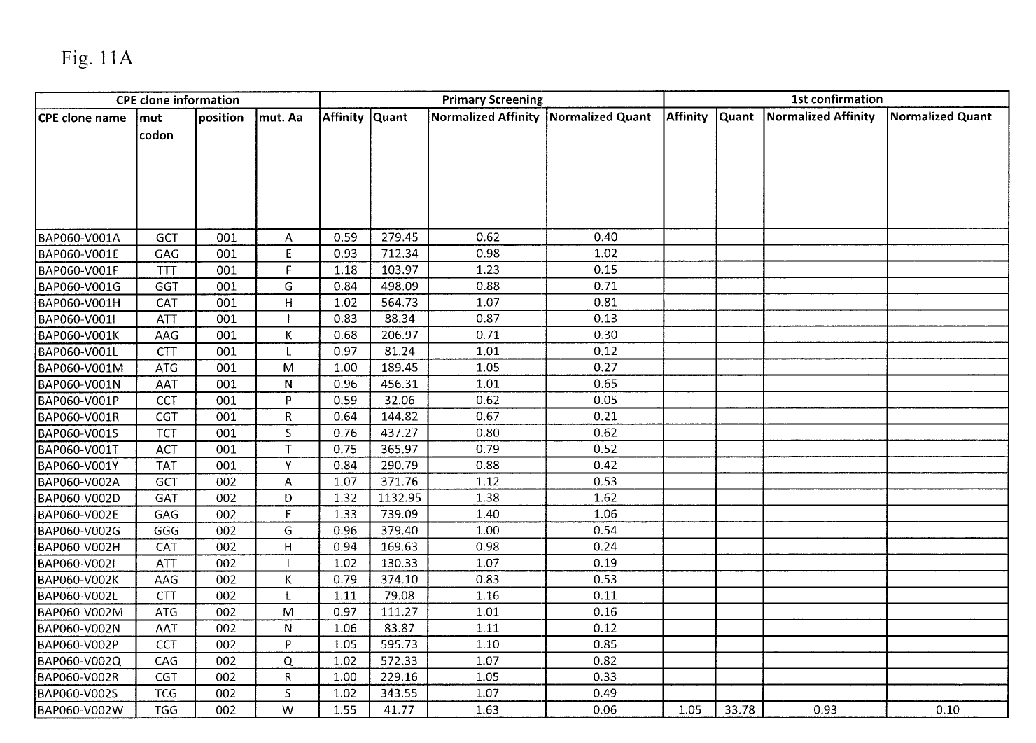

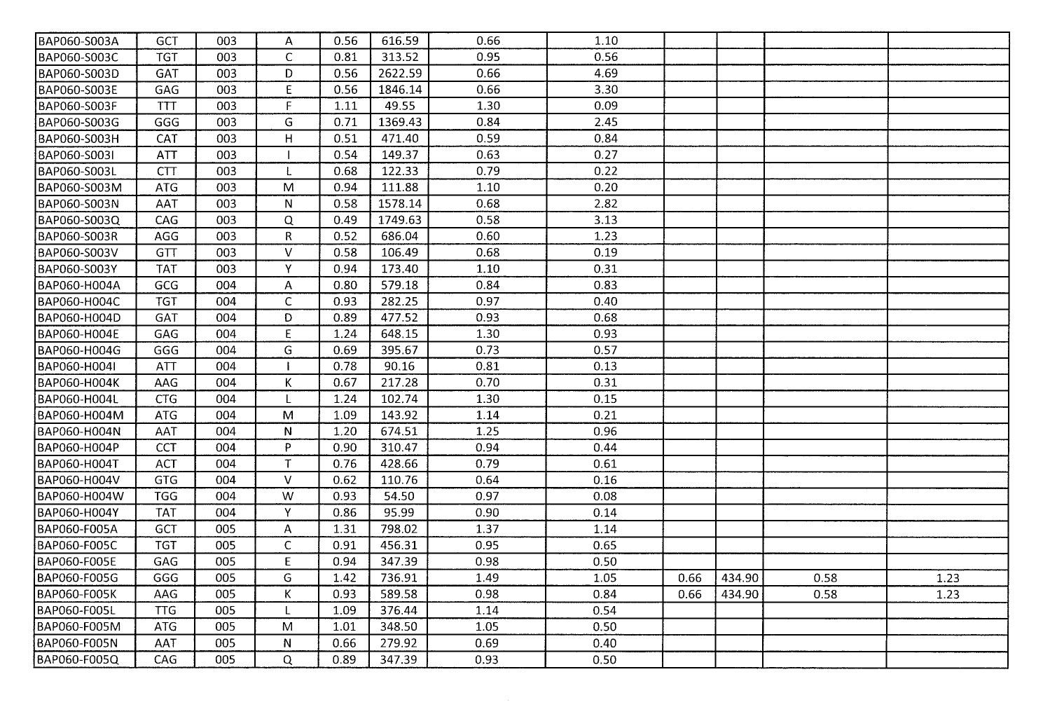

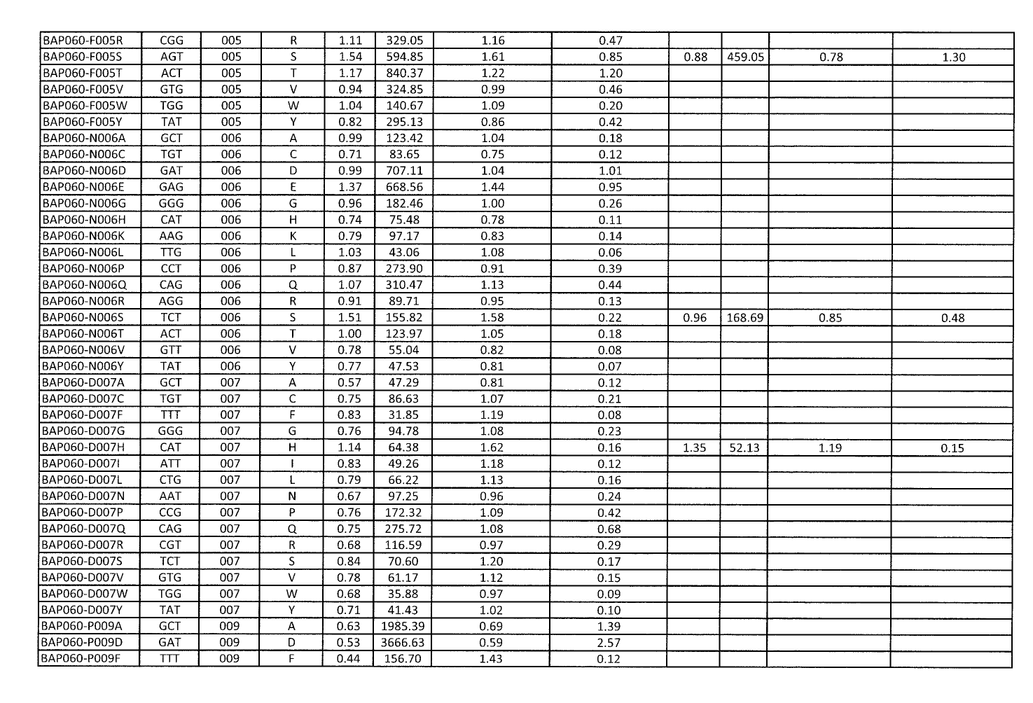

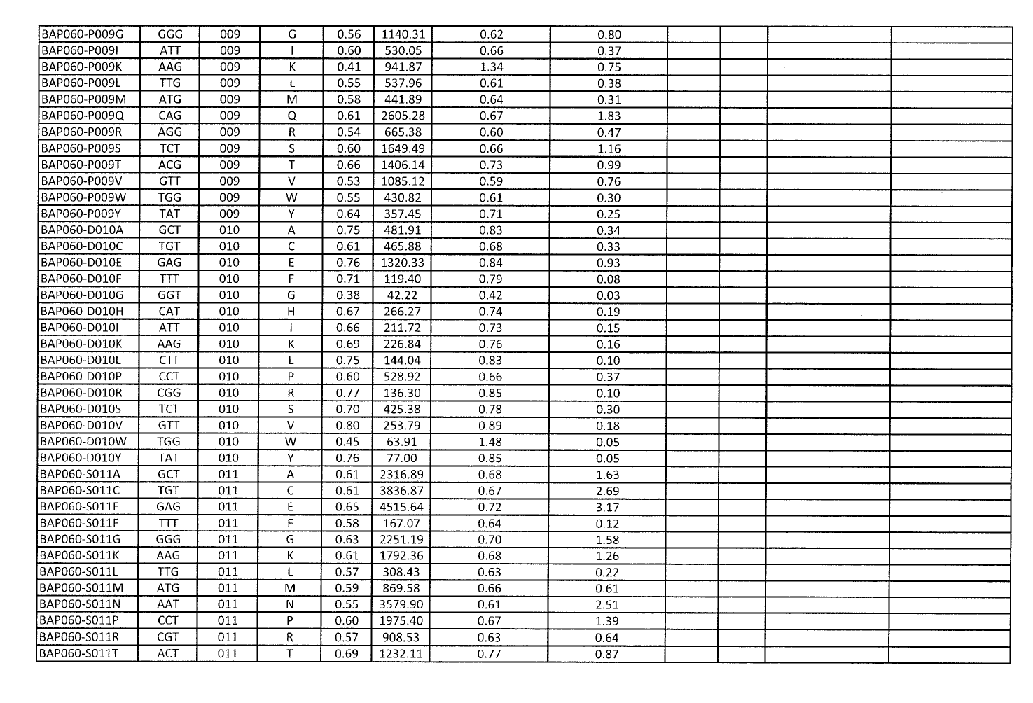

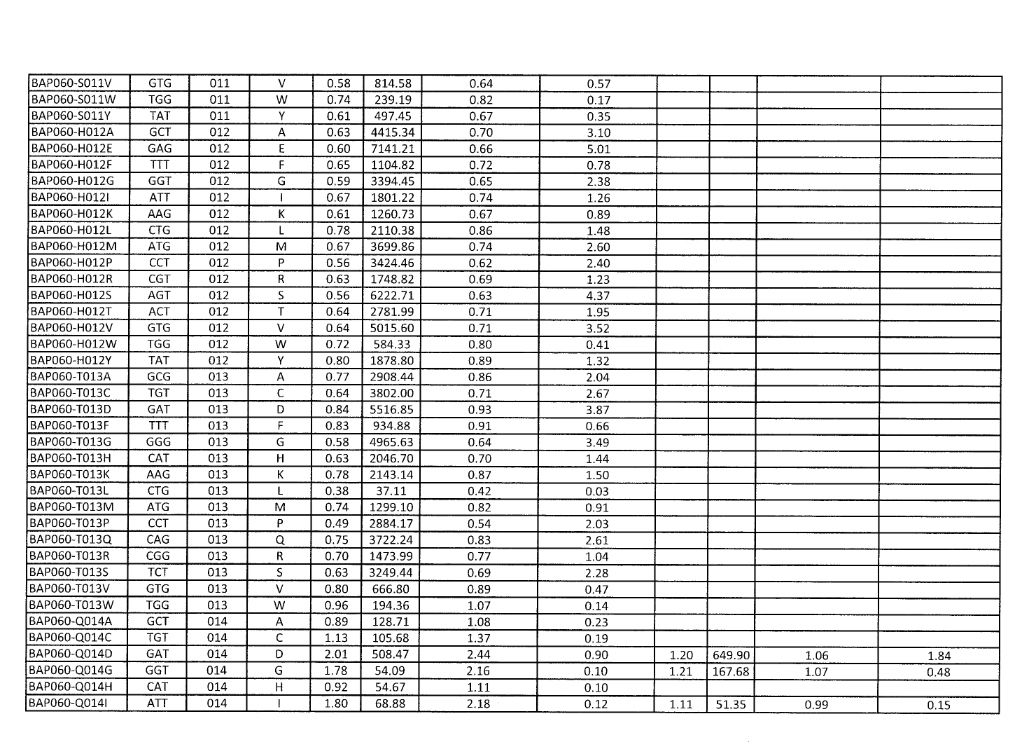

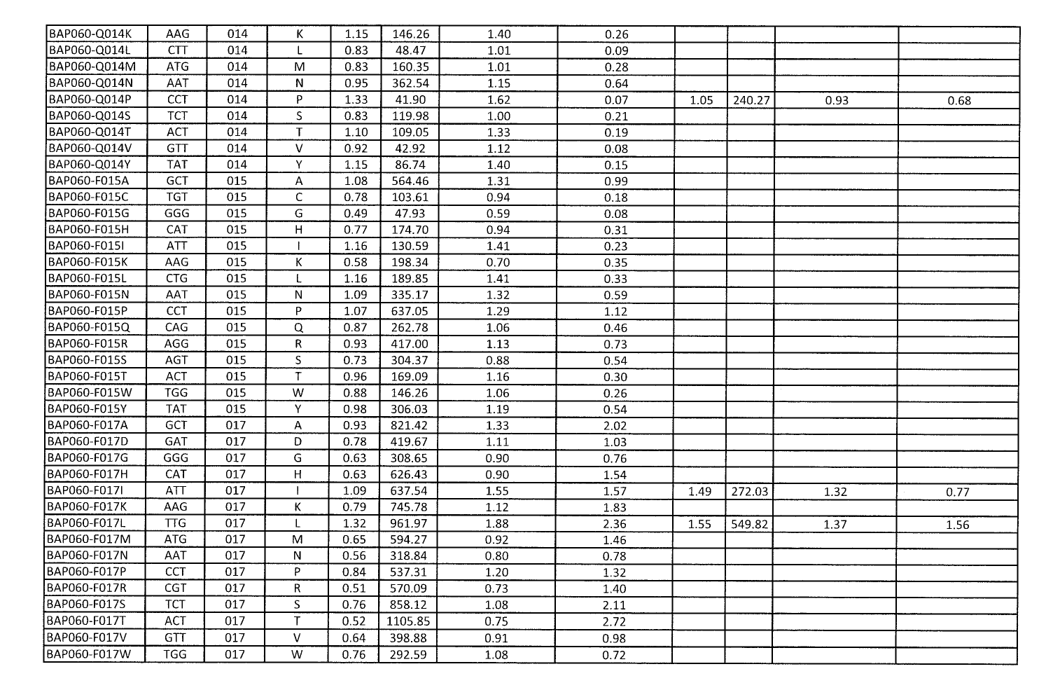

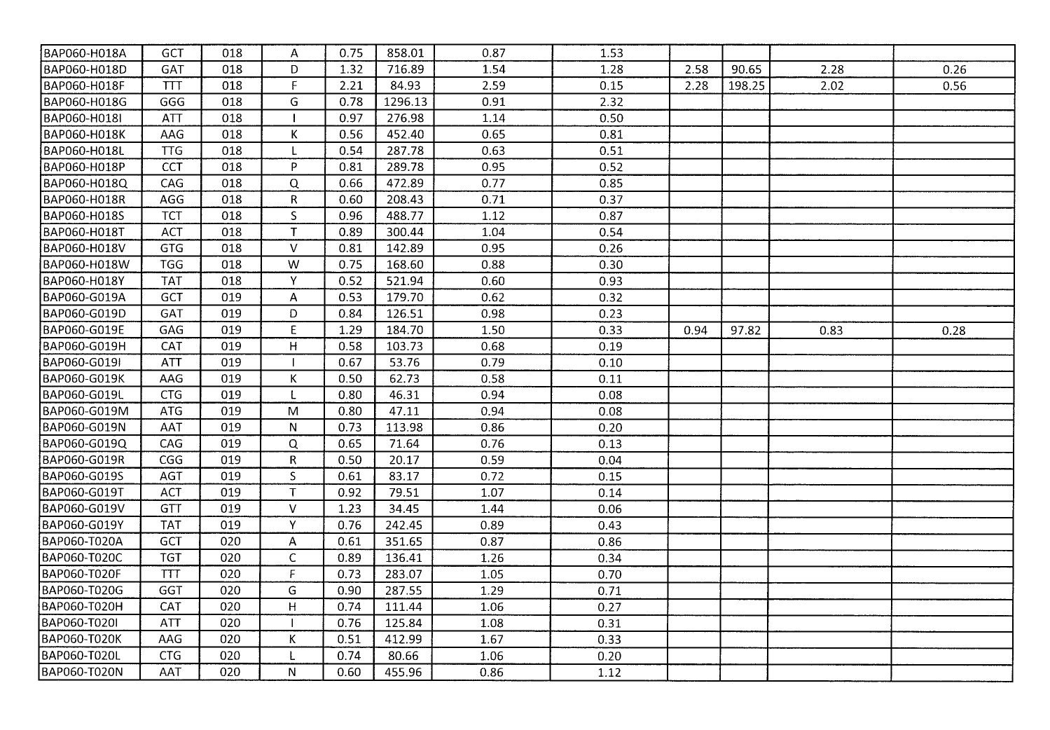

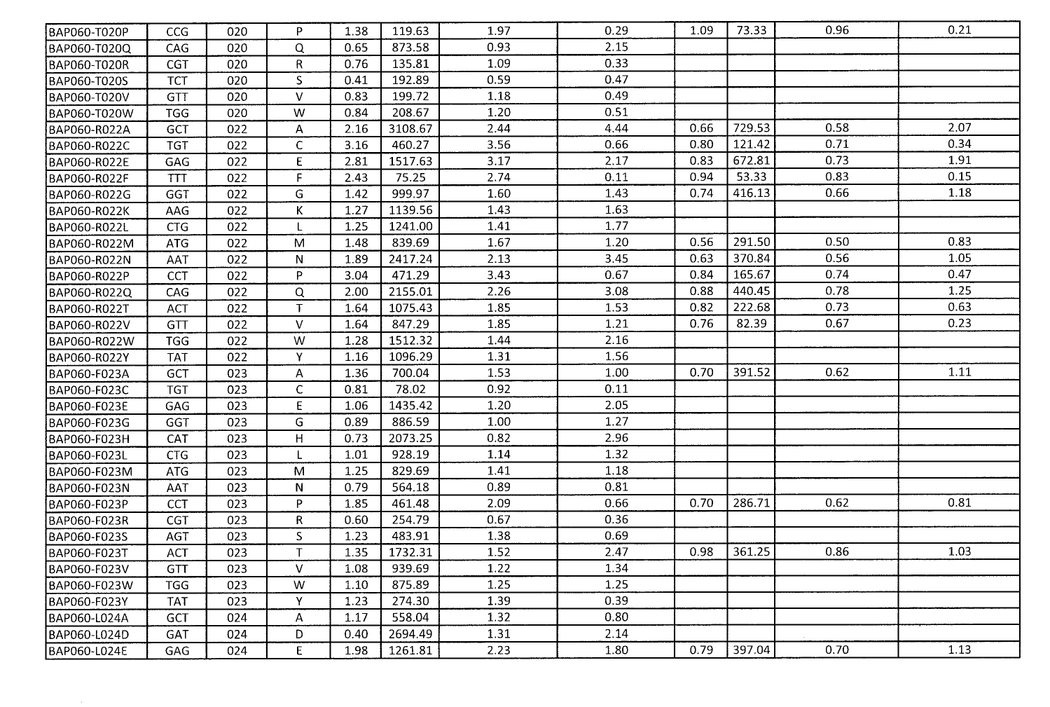

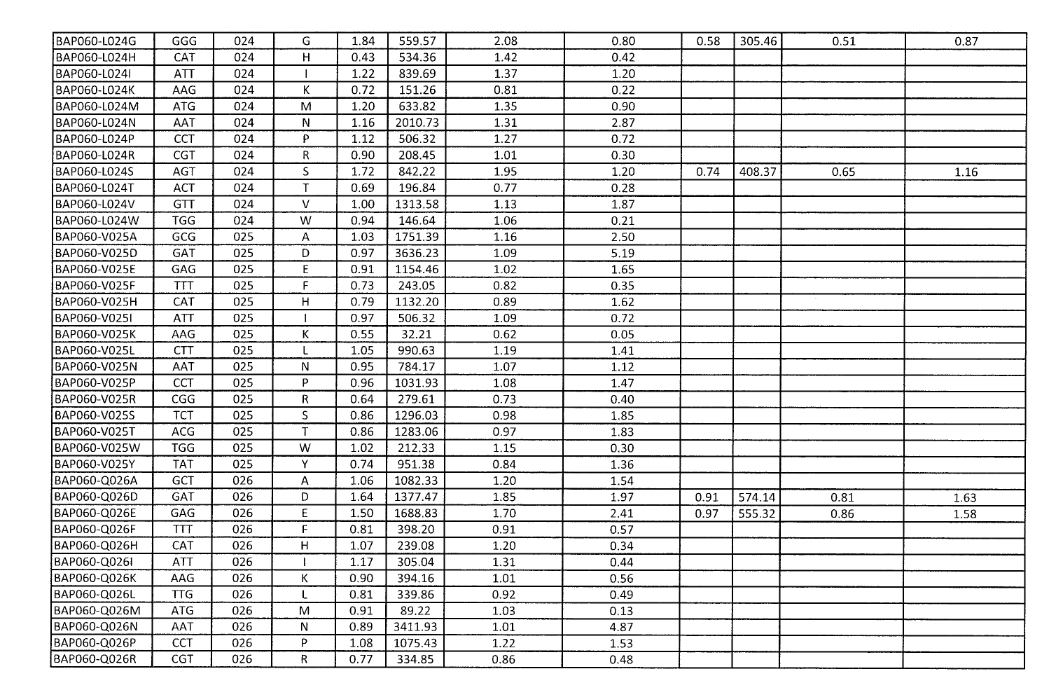

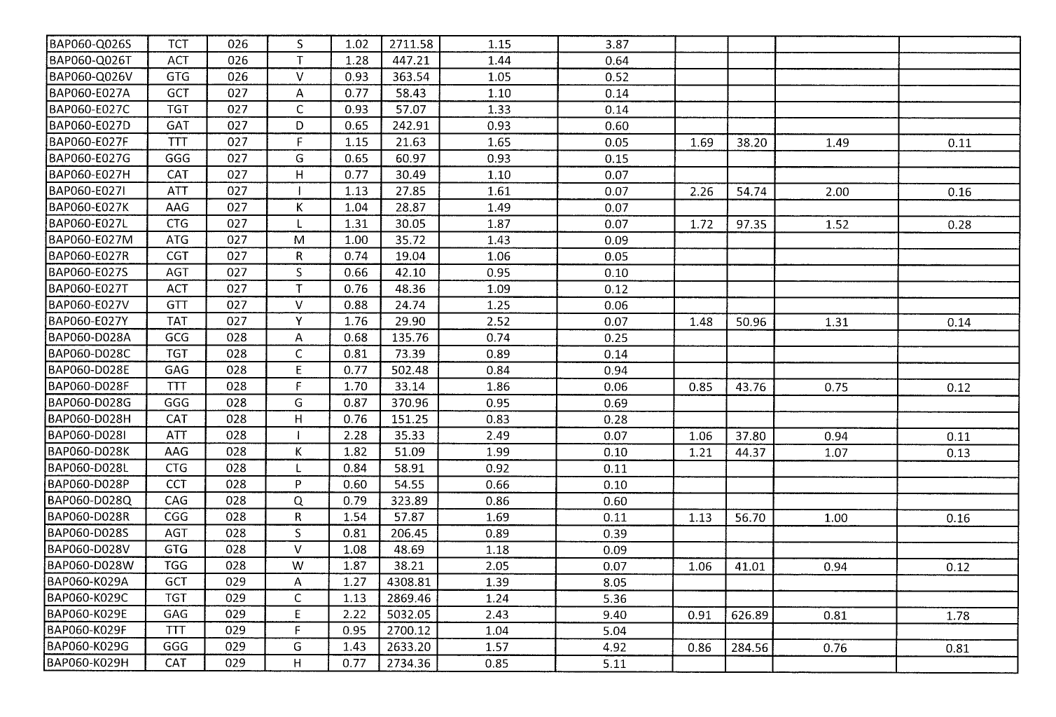

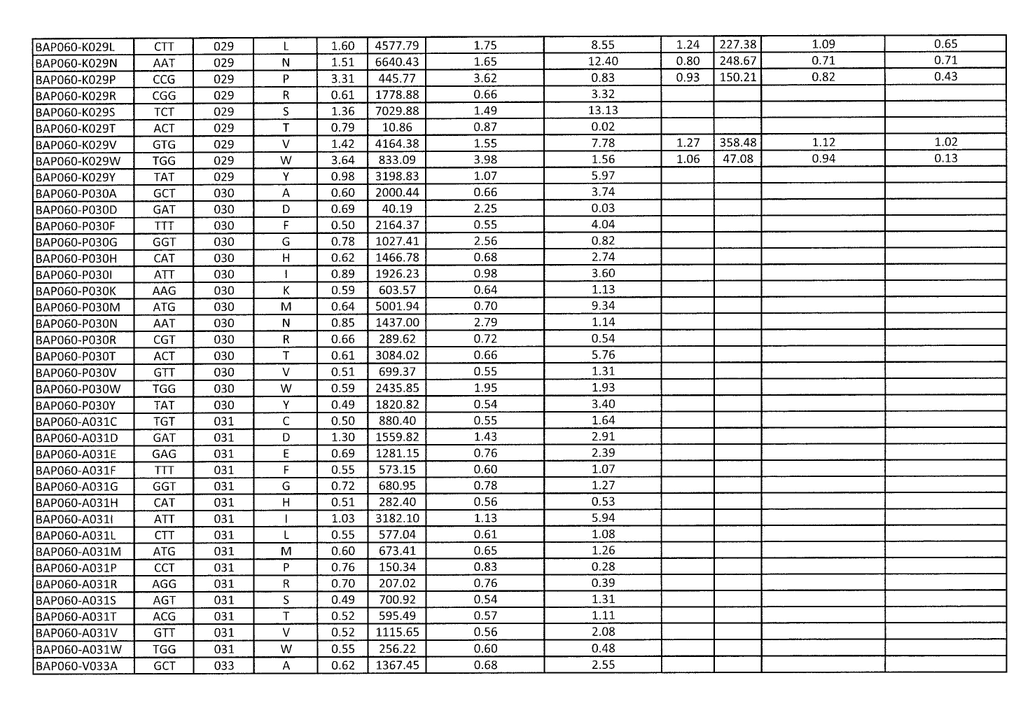

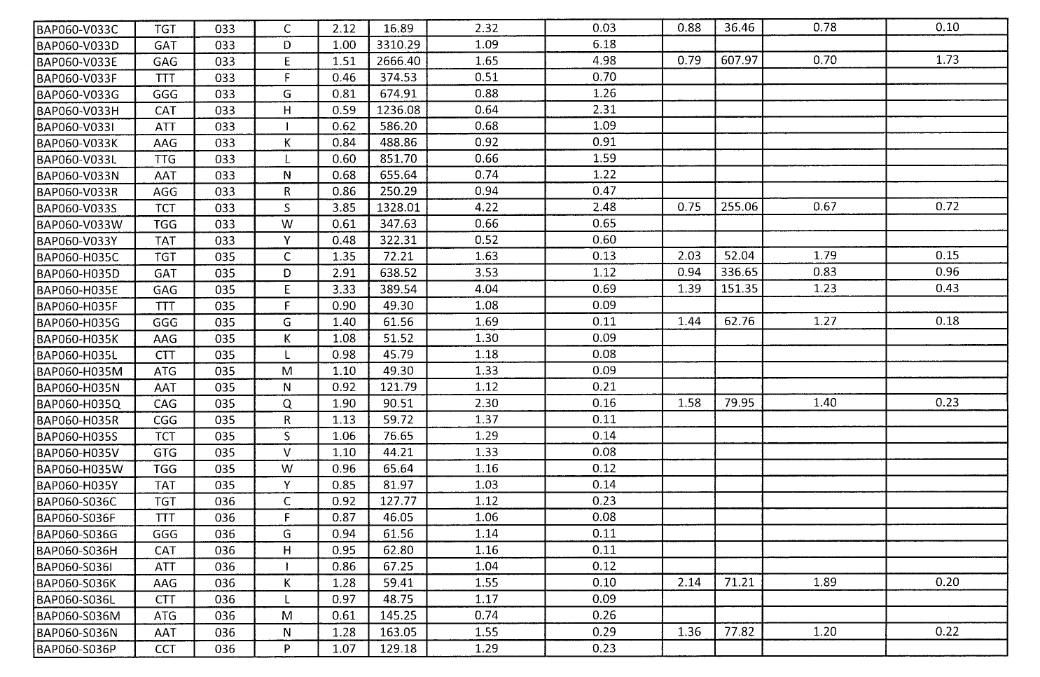

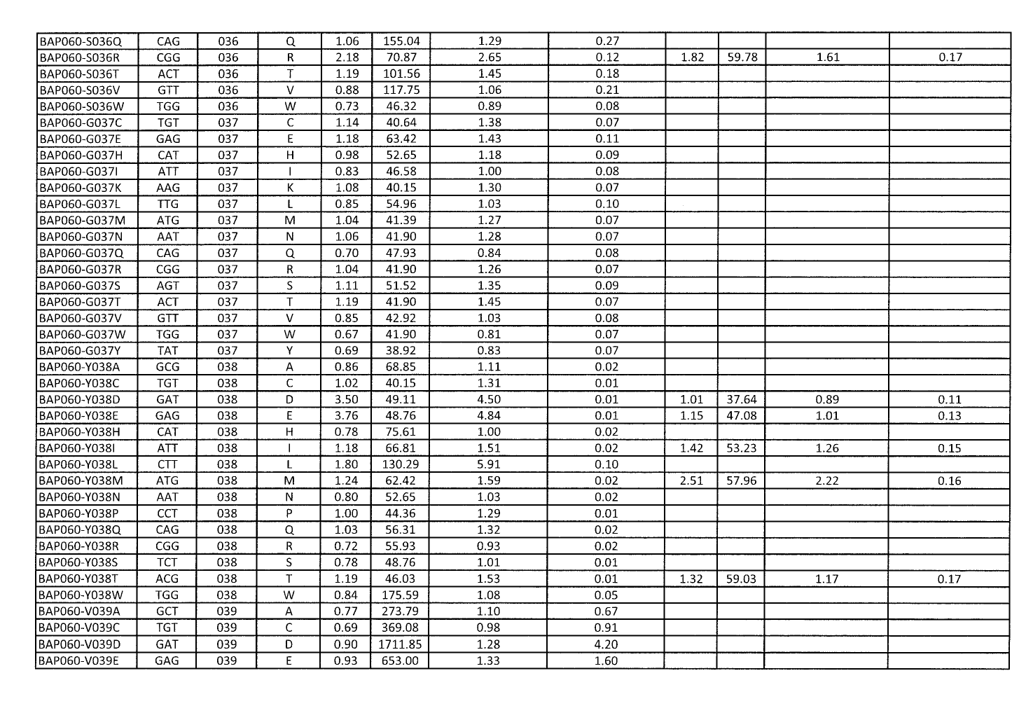

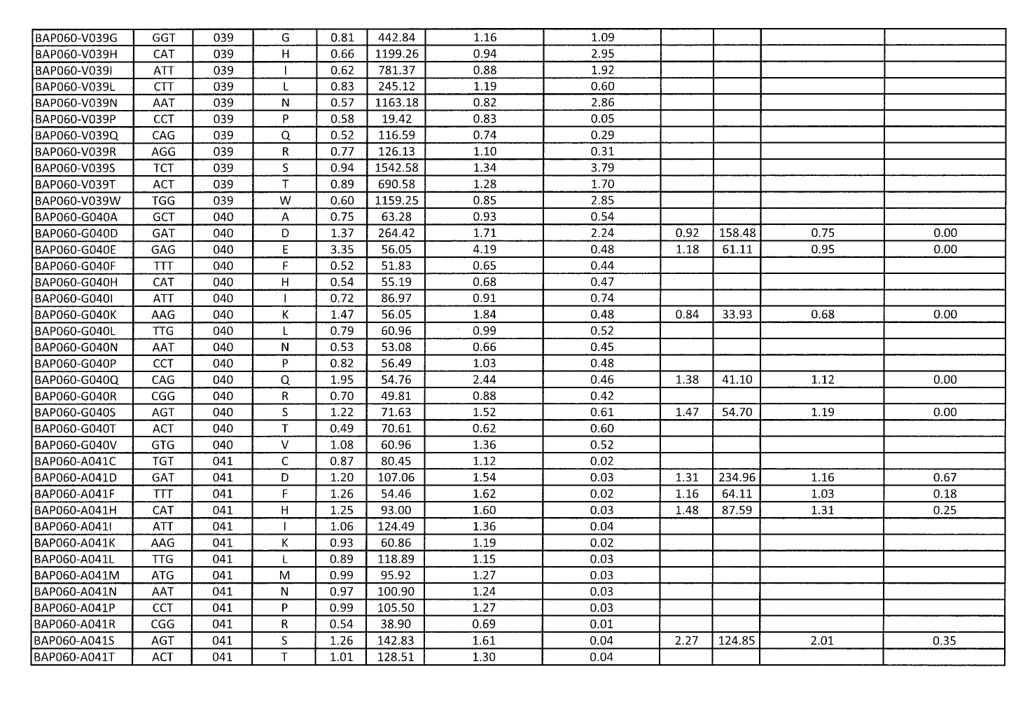

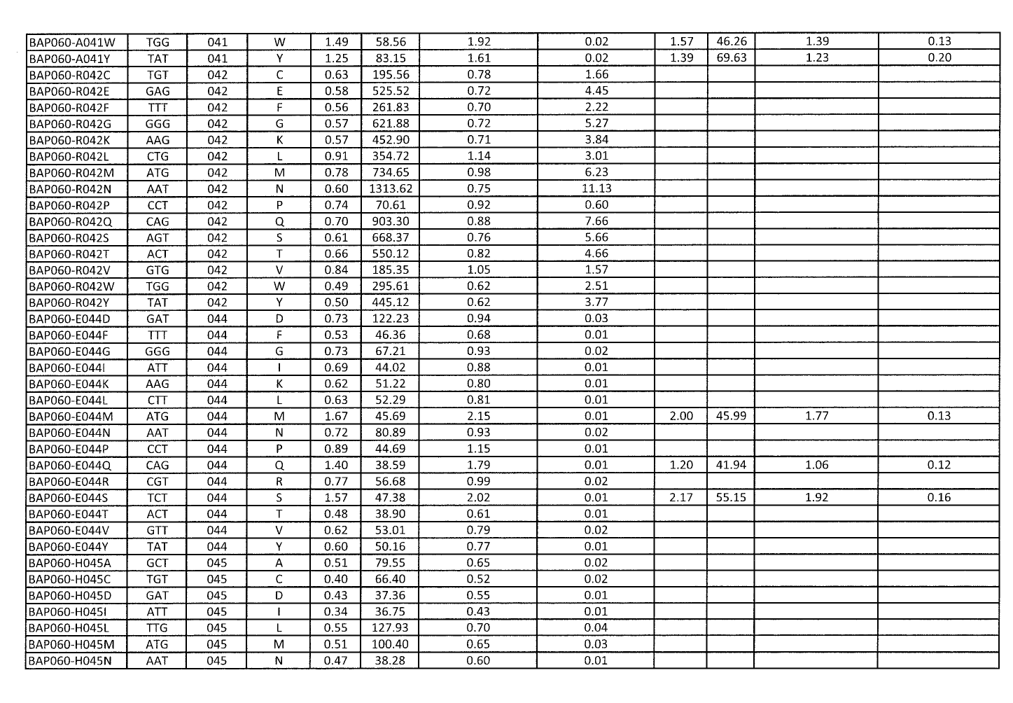

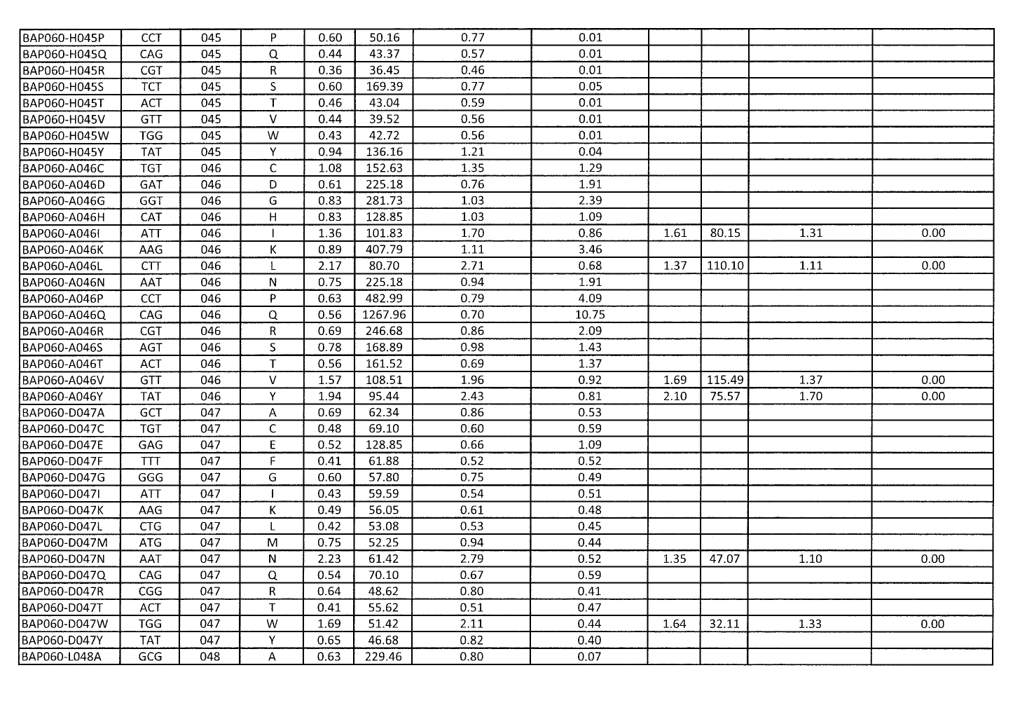

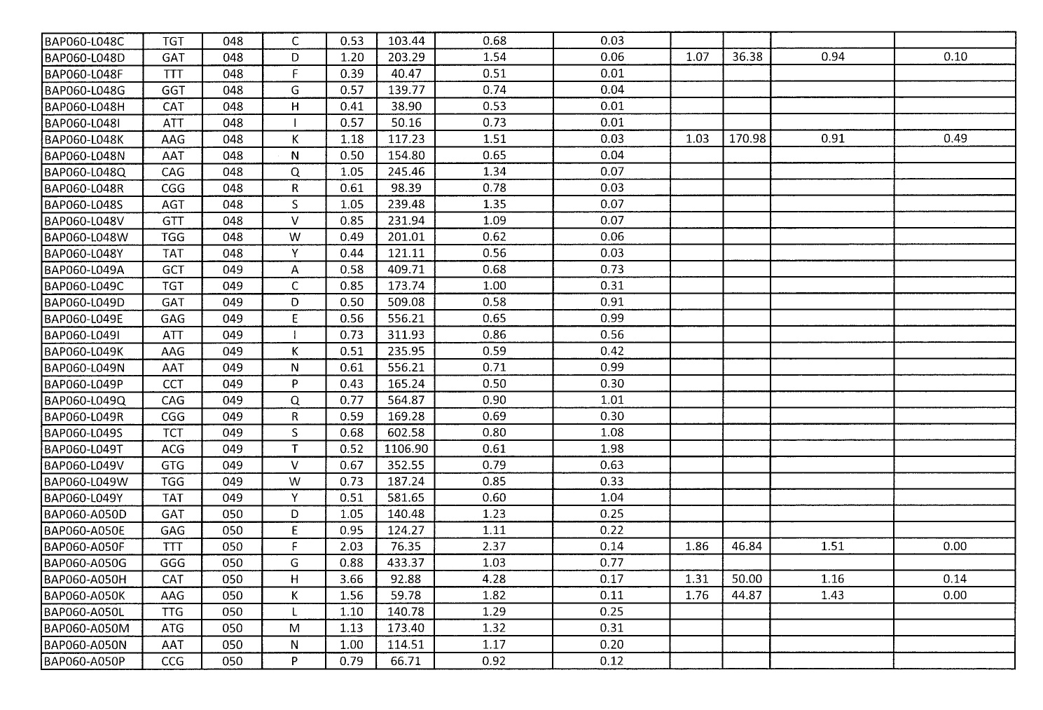

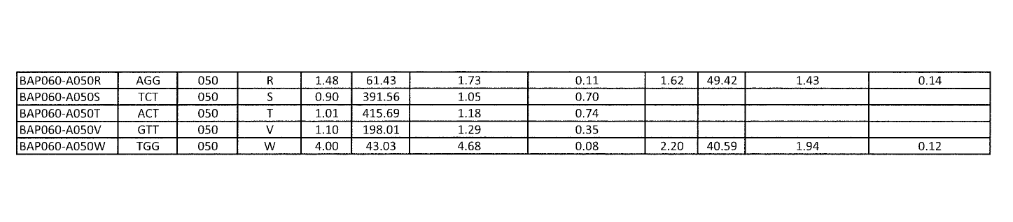

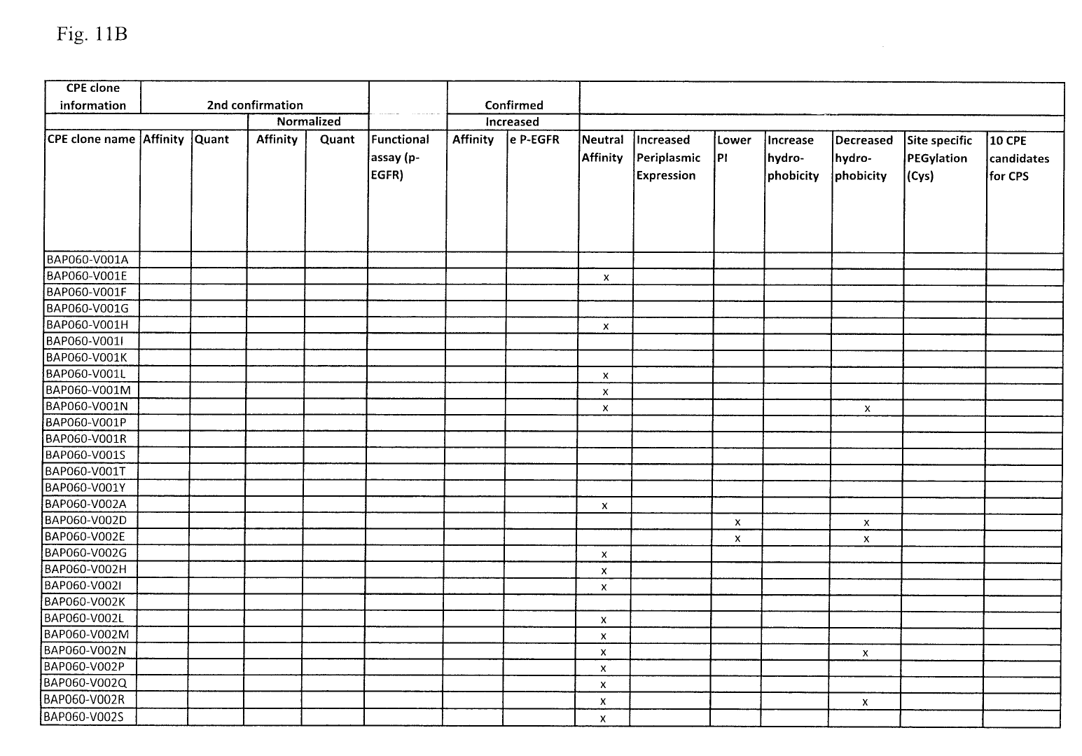

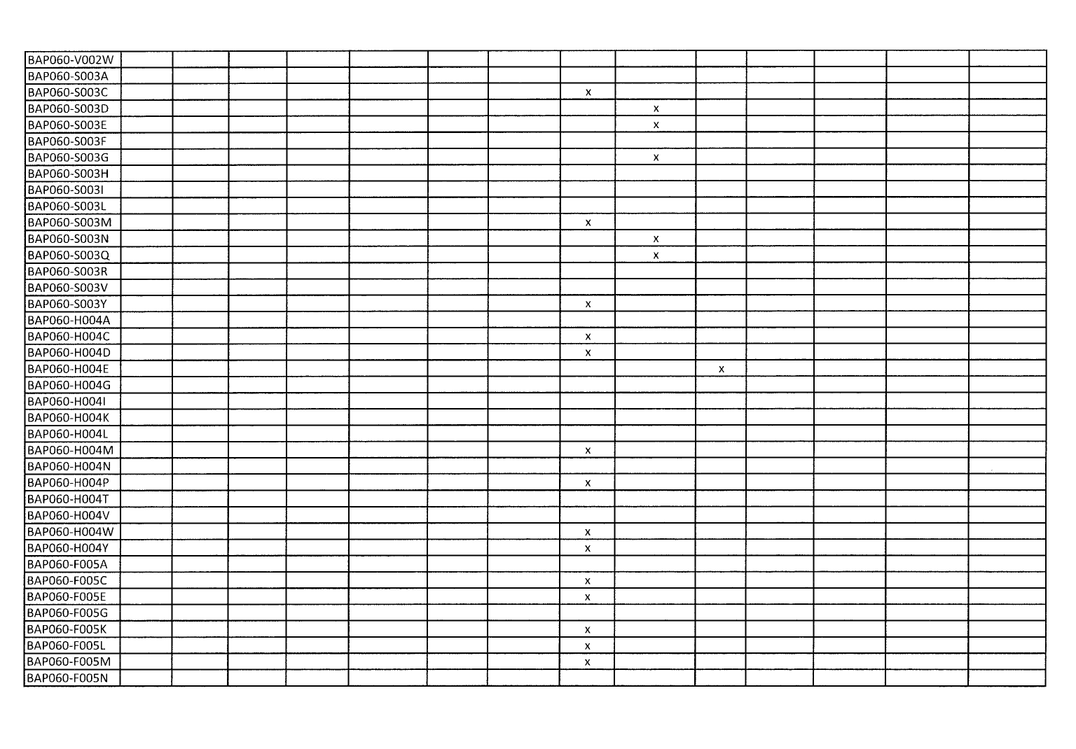

The activity of hTGFα expressed in the periplasmic fractions of BL21 cells at 37° C. was measured using cell based ELISA and FACS analysis. Expression of recombinant hTGFα and preparation of periplasmic fractions was conducted as described above. The bacterially expressed hTGFα was active in both assays and its activity was comparable to the commercially available hTGFα (FIGS. 8A-C and 9A & B). In addition, the activity of hTGFα expressed in BL21 cells was tested using a cell based EGFR phosphorylation assay to determine the binding activity of hTGFα to EGFR. The results showed that the bacterially expressed hTGFα can bind and activate EGFR on the cell surface (FIG. 10A-D).

3. Expression of hTGFα in CHO Cells and Determination of Activity

CHO cells were seeded into 96-well plates and transfected with constructs BAP060-hTGFα-M and BAP060-mhTGFα-M, (see Table 1) using lipofectamine 2000 (Invitrogen) and cultured at 37° C. in CD-CHO serum free media. Supernatants were collected at 48 hours post transfection. Two fold serially diluted hTGFα standards starting at 20 ng/ml (FIG. 7A, columns 1 and 2), 1:5 or 1:20 diluted bacterially expressed hTGFα (FIG. 7A, column 3) and two fold serially diluted mammalian expressed hTGFα (CHO cells transfected with BAP060 mhTGFα-M, FIG. 7A, column 4, or CHO cells transfected with BAP060-hTGFα -M, FIG. 7A, column 5) were incubated with anti-TGFα (R&D Systems, #AF-239-NA) immobilized on the plate. Bound TGFα was detected with biotinylated anti-TGFα (R&D Systems, #BAF239) and Streptavidin conjugated with HRP. The reactions were stopped at 3 minute after TMB was added to the wells and read immediately. OD450 nm value of the reactions was measured with Molecular Device SPECTRAmax Plus. The standard curve was generated using Softmax.

The results indicate that the hTGFα constructs are expressed in CHO cells, processed correctly and secreted into media, although at lower level compare to the bacterially expressed hTGFα (FIG. 7B). The activity of hTGFα expressed in CHO cells was measured using cell based ELISA and FACS analysis. The mammalian expressed hTGFα was active and its activity was comparable to the commercially available hTGFα (FIGS. 8A-C and 9A& B). In addition, the activity of hTGFα expressed in CHO cells was also tested using a cell based EGFR phosphorylation assay to determine the binding activity of hTGFα to EGFR. The results showed that the mammalian expressed hTGFα can bind and activate EGFR on the cell surface (FIG. 10A-D).

4. Adaptation of the hTGFα Expression Process in BL21 to the 96 Well Format for High Throughput Screening

Bacterially expressed hTGFα construct was used as the template for the CPE™ process since the expression level of soluble hTGFα in bacteria is higher than the expression level of hTGFα in CHO cells. In addition, bacterially expressed hTGFα is functional and has comparable binding activity compared to the commercially available hTGFα. The expression process in bacteria was adapted in 96 well format and it was confirmed that the expression level of hTGFα prepared in the 96 well blocks was comparable to that of hTGFα prepared in the 14 ml tubes measured by quantitation ELISA. Finally, the activity of hTGFα expressed in the 96 well format was measured by cell based ELISA, FACS analysis and EGFR phosphorylation assay, and activity of hTGFα expressed in the 96 well format was found to be comparable to that of the hTGFα expressed in 14 ml tube format (data not shown).

### Example 2

### This Examples describes the Construction of Novel TGFα Mutants, hTGFα Evolution with CPE™

1. Construction of the CPE™ Library of Human TGFα

A Comprehensive Positional Evolution (CPE™) library of BAP060-hTGFα -B (SEQ ID NO:145) having 15 amino acid variants at each position except for position 30 which has 14 amino acid variants was constructed. The amino acid sequence shown below is the mature, human, TGFα sequence and the numbering reflects the numbering used for the CPE™ residues:

1 10 20 30 40 50
VVSHFNDCPD SHTQFCFHGT CRFLVQEDKP ACVCHSGYVG ARCEHADLLA

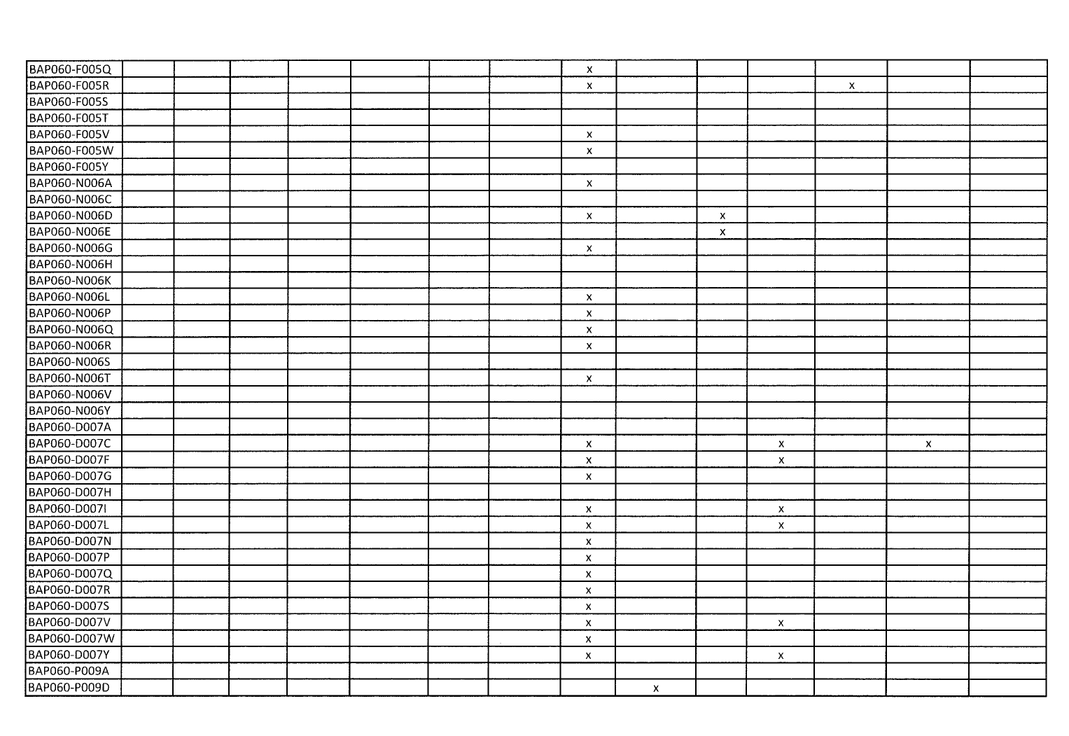

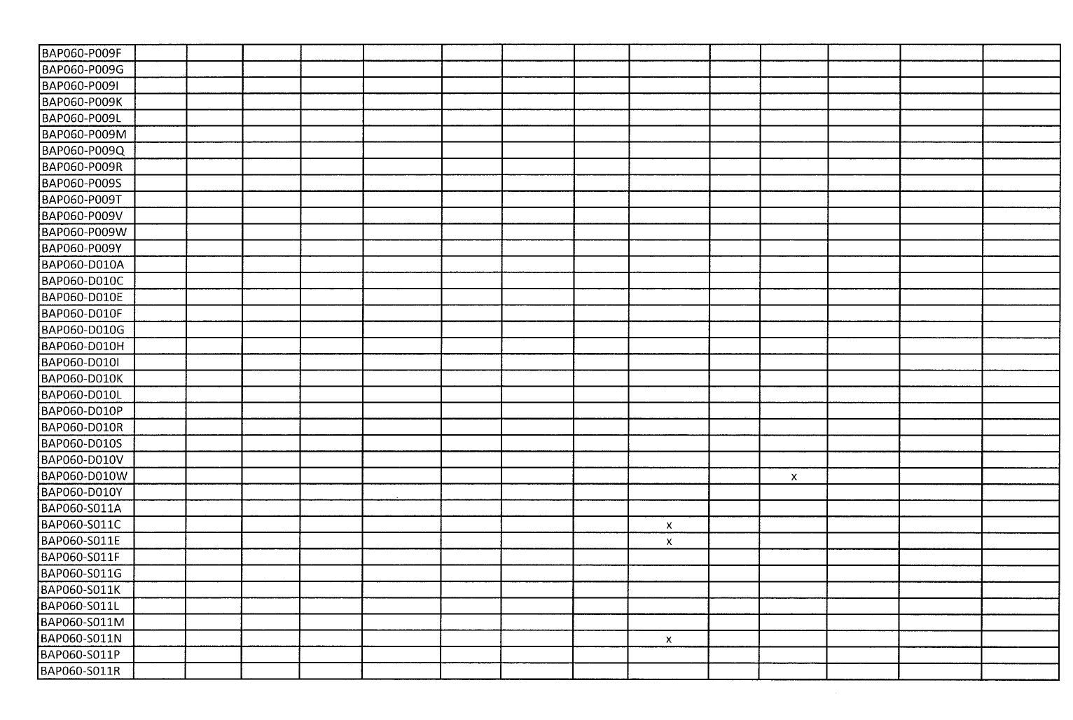

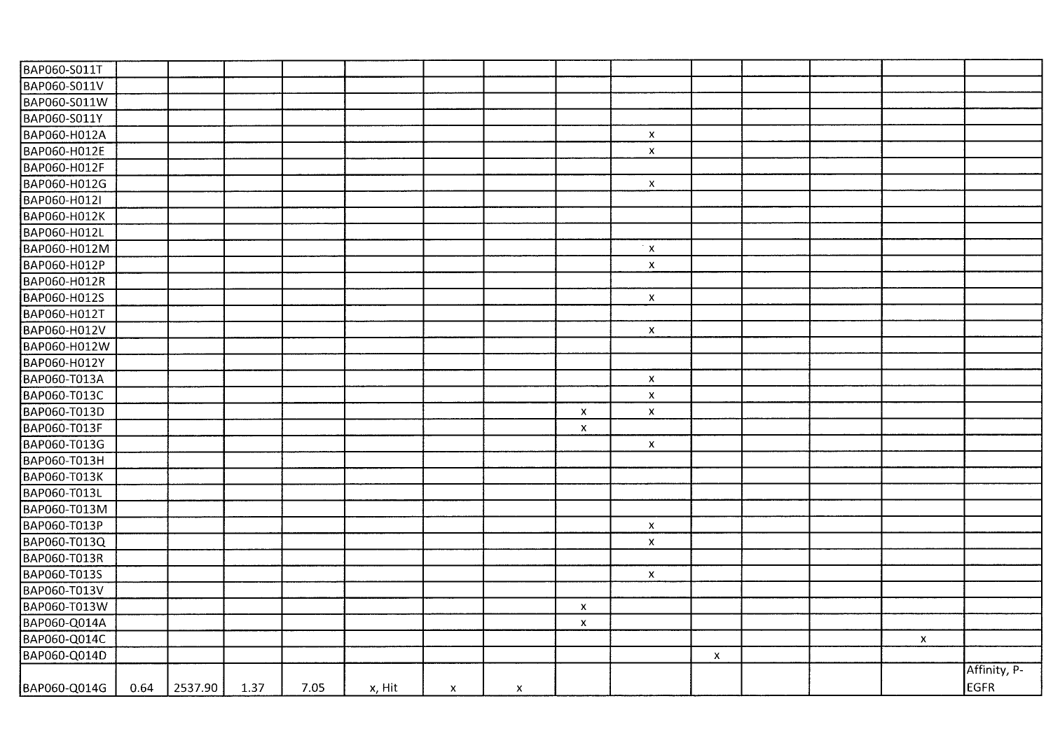

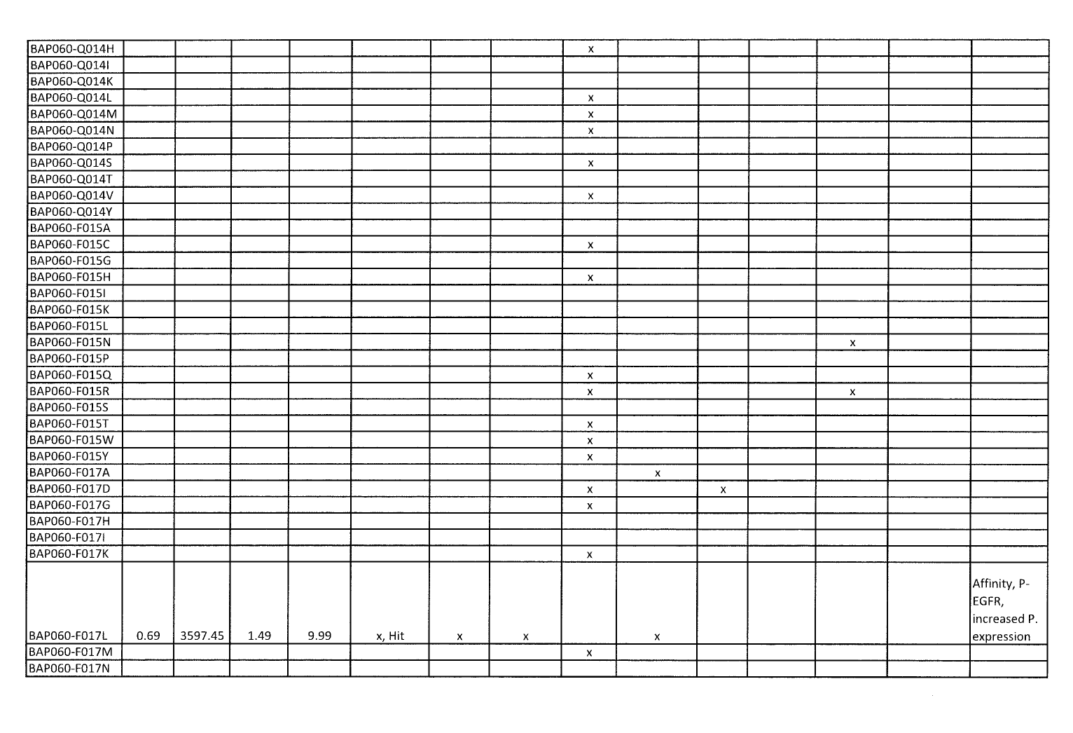

All of the amino acids listed above were included in the CPE™ process, with the exception of the cysteine residues at positions 8, 16, 21, 32, 34 and 43 (each underlined). These residues form structurally important disulfide bonds. The final CPE™ library contains 660 BAP060-hTGFα -B mutants arrayed in 96-well format. Details on the mutation in each mutant are shown in FIG. 11A.

2. Expression of hTGFα CPE™ Mutants in E. coli BL21 (DE3) Cells and Determination of Binding Activity

BAP060-hTGFα-B (mature hTGFα, aa 40-89, with PelB signal sequence) wild type and CPE™ mutants were transformed into E. coli BL21 (DE3) competent cells, and recombinant protein expression induced by addition of 1 mM IPTG at 37° C. for 3 hours in 96 well format (1st confirmation) or in 14 ml tubes (2nd confirmation). After three hours, the cells were collected and periplasmic proteins prepared by incubating the cells in OS buffer (200 mM Tris-HCl, pH 7.5, 20% sucrose, 1 mM EDTA) with DNAse, RNAse and lysozyme. After addition of lysis buffer (10 mM Tris, pH 7.5, 50 mM KCl, 1 mM EDTA and 0.1% of Na-deoxycholate) to the cells in OS buffer, the cells were centrifuged and the supernatants, which contained the periplasmic fractions, were transferred to microcentrifuge tubes. The concentration of hTGFα was determined by quantitation ELISA, and 25 ng/ml of hTGFα were incubated with A431 cells (ATCC#CRL1555) immobilized on the plate. Bound hTGFα was detected with biotinylated anti-hTGFα (R&D Systems, #BAF239) and Streptavidin conjugated with HRP. The reactions were stopped by addition of HCl after TMB was added to the wells and read immediately. OD450 nm value of the reactions was measured with a Molecular Device SPECTRAmax. The results of this initial screen are shown in FIG. 11A. The initial screen identified 94 CPE™ mutants that had 1.5 fold higher binding activity compared to the wild type TGFα in the primary screening. The sequences of these CPE™ mutants are shown below in Table 2, while select characteristics for all of the CPE™ mutants can be found in FIG. 11A.

TABLE 2
Sequence of WT and Mutant TGFa Proteins

| SEQ ID | CPE ™ Clone | |
|---|---|---|
| NO: | Name | Sequence |
| 1 | WildType | VVSHFNDCPDSHTQFCFHGTCRFLVQEDKPACVCHSGYVGARCEHADLLA |
| 2 | BAP060-V002W | VWSHFNDCPDSHTQFCFHGTCRFLVQEDKPACVCHSGYVGARCEHADLLA |
| 3 | BAP060-F005G | VVSHGNDCPDSHTQFCFHGTCRFLVQEDKPACVCHSGYVGARCEHADLLA |
| 4 | BAP060-E005K | VVSHKNDCPDSHTQFCFHGTCRFLVQEDKPACVCHSGYVGARCEHADLLA |
| 5 | BAP060-F005S | VVSHSNDCPDSHTQFCFHGTCRFLVQEDKPACVCHSGYVGARCEHADLLA |
| 6 | BAP060-N006S | VVSHFSDCPDSHTQFCFHGTCRFLVQEDKPACVCHSGYVGARCEHADLLA |
| 7 | BAP060-D007H | VVSHFNHCPDSHTQFCFHGTCRFLVQEDKPACVCHSGYVGARCEHADLLA |
| 8 | BAP060-Q014D | VVSHENDCPDSHTDFCFHGTCRFLVQEDKPACVCHSGYVGARCEHADLLA |
| 9 | BAP060-Q014G | VVSHFNDCPDSHTGFCFHGTCRFLVQEDKPACVCHSGYVGARCEHADLLA |
| 10 | BAP060-Q014I | VVSHFNDCPDSHTIFCFHGTCRFLVQEDKPACVCHSGYVGARCEHADLLA |
| 11 | BAP060-Q014P | VVSHENDCPDSHTPFCFHGTCRFLVQEDKPACVCHSGYVGARCEHADLLA |
| 12 | BAP060-F017I | VVSHFNDCPDSHTQFCIHGTCRFLVQEDKPACVCHSGYVGARCEHADLLA |
| 13 | BAP060-F017L | VVSHFNDCPDSHTQFCLHGTCRFLVQEDKPACVCHSGYVGARCEHADLLA |
| 14 | BAP060-H018D | VVSHENDCPDSHTQFCFDGTCRFLVQEDKPACVCHSGYVGARCEHADLLA |
| 15 | BAP060-H018F | VVSHFNDCPDSHTQFCFFGTCRFLVQEDKPACVCHSGYVGARCEHADLLA |
| 16 | BAP060-G019E | VVSHFNDCPDSHTQFCFHETCRFLVQEDKPACVCHSGYVGARCEHADLLA |
| 17 | BAP060-T020P | VVSHFNDCPDSHTQFCFHGPCRFLVQEDKPACVCHSGYVGARCEHADLLA |
| 18 | BAP060-R022A | VVSHFNDCPDSHTQFCFHGTCAFLVQEDKPACVCHSGYVGARCEHADLLA |
| 19 | BAP060-R022C | VVSHFNDCPDSHTQFCFHGTCCFLVQEDKPACVCHSGYVGARCEHADLLA |
| 20 | BAP060-R022E | VVSHFNDCPDSHTQFCFHGTCEFLVQEDKPACVCHSGYVGARCEHADLLA |
| 21 | BAP060-R022F | VVSHFNDCPDSHTQFCFHGTCFFLVQEDKPACVCHSGYVGARCEHADLLA |
| 22 | BAP060-R022G | VVSHFNDCPDSHTQFCFHGTCGFLVQEDKPACVCHSGYVGARCEHADLLA |
| 23 | BAP060-R022M | VVSHFNDCPDSHTQFCFHGTCMFLVQEDKPACVCHSGYVGARCEHADLLA |
| 24 | BAP060-R022N | VVSHFNDCPDSHTQFCFHGTCNFLVQEDKPACVCHSGYVGARCEHADLLA |
| 25 | BAP060-R022P | VVSHFNDCPDSHTQFCFHGTCPFLVQEDKPACVCHSGYVGARCEHADLLA |
| 26 | BAP060-R022Q | VVSHENDCPDSHTQFCFHGTCQFLVQEDKPACVCHSGYVGARCEHADLLA |
| 27 | BAP060-R022T | VVSHFNDCPDSHTQFCFHGTCTFLVQEDKPACVCHSGYVGARCEHADLLA |
| 28 | BAP060-R022V | VVSHFNDCPDSHTQFCFHGTCVFLVQEDKPACVCHSGYVGARCEHADLLA |
| 29 | BAP060-F023A | VVSHFNDCPDSHTQFCFHGTCRALVQEDKPACVCHSGYVGARCEHADLLA |
| 30 | BAP060-E023P | VVSHENDCPDSHTQFCFHGTCRPLVQEDKPACVCHSGYVGARCEHADLLA |
| 31 | BAP060-F023T | VVSHFNDCPDSHTQFCFHGTCRTLVQEDKPACVCHSGYVGARCEHADLLA |
| 32 | BAP060-L024E | VVSHFNDCPDSHTQFCFHGTCRFEVQEDKPACVCHSGYVGARCEHADLLA |
| 33 | BAP060-L024G | VVSHFNDCPDSHTQFCFHGTCRFGVQEDKPACVCHSGYVGARCEHADLLA |
| 34 | BAP060-L024S | VVSHENDCPDSHTQFCFHGTCRFSVQEDKPACVCHSGYVGARCEHADLLA |
| 35 | BAP060-Q026D | VVSHENDCPDSHTQFCFHGTCRFLVDEDKPACVCHSGYVGARCEHADLLA |
| 36 | BAP060-Q026E | VVSHFNDCPDSHTQFCFHGTCRFLVEEDKPACVCHSGYVGARCEHADLLA |
| 37 | BAP060-E027F | VVSHFNDCPDSHTQFCFHGTCRFLVQFDKPACVCHSGYVGARCEHADLLA |
| 38 | BAP060-E027I | VVSHENDCPDSHTQFCFHGTCRFLVQIDKPACVCHSGYVGARCEHADLLA |
| 39 | BAP060-E027L | VVSHFNDCPDSHTQFCFHGTCRFLVQLDKPACVCHSGYVGARCEHADLLA |
| 40 | BAP060-E027Y | VVSHFNDCPDSHTQFCFHGTCRFLVQYDKPACVCHSGYVGARCEHADLLA |
| 41 | BAP060-D028F | VVSHFNDCPDSHTQFCFHGTCRFLVQEFKPACVCHSGYVGARCEHADLLA |
| 42 | BAP060-D028I | VVSHFNDCPDSHTQFCFHGTCRFLVQEIKPACVCHSGYVGARCEHADLLA |
| 43 | BAP060-D028K | VVSHENDCPDSHTQFCFHGTCRFLVQEKKPACVCHSGYVGARCEHADLLA |
| 44 | BAP060-D028R | VVSHFNDCPDSHTQFCFHGTCRFLVQERKPACVCHSGYVGARCEHADLLA |
| 45 | BAP060-D028W | VVSHFNDCPDSHTQFCFHGTCRFLVQEWKPACVCHSGYVGARCEHADLLA |
| 46 | BAP060-K029E | VVSHFNDCPDSHTQFCFHGTCRFLVQEDEPACVCHSGYVGARCEHADLLA |
| 47 | BAP060-K029G | VVSHFNDCPDSHTQFCFHGTCRFLVQEDGPACVCHSGYVGARCEHADLLA |
| 48 | BAP060-K029L | VVSHFNDCPDSHTQFCFHGTCRFLVQEDLPACVCHSGYVGARCEHADLLA |
| 49 | BAP060-K029N | VVSHFNDCPDSHTQFCFHGTCRFLVQEDNPACVCHSGYVGARCEHADLLA |
| 50 | BAP060-K029P | VVSHENDCPDSHTQFCFHGTCRFLVQEDPPACVCHSGYVGARCEHADLLA |
| 51 | BAP060-K029V | VVSHFNDCPDSHTQFCFHGTCRFLVQEDVPACVCHSGYVGARCEHADLLA |
| 52 | BAP060-K029W | VVSHFNDCPDSHTQFCFHGTCRFLVQEDWPACVCHSGYVGARCEHADLLA |
| 53 | BAP060-V033C | VVSHFNDCPDSHTQFCFHGTCRFLVQEDKPACCCHSGYVGARCEHADLLA |
| 54 | BAP060-V033E | VVSHFNDCPDSHTQFCFHGTCRFLVQEDKPACECHSGYVGARCEHADLLA |
| 55 | BAP060-V033S | VVSHFNDCPDSHTQFCFHGTCRFLVQEDKPACSCHSGYVGARCEHADLLA |
| 56 | BAP060-H035C | VVSHFNDCPDSHTQFCFHGTCRFLVQEDKPACVCCSGYVGARCEHADLLA |
| 57 | BAP060-H035D | VVSHFNDCPDSHTQFCFHGTCRFLVQEDKPACVCDSGYVGARCEHADLLA |
| 58 | BAP060-H035E | VVSHFNDCPDSHTQFCFHGTCRFLVQEDKPACVCESGYVGARCEHADLLA |
| 59 | BAP060-H035G | VVSHFNDCPDSHTQFCFHGTCRFLVQEDKPACVCGSGYVGARCEHADLLA |
| 60 | BAP060-H035Q | VVSHFNDCPDSHTQFCFHGTCRFLVQEDKPACVCQSGYVGARCEHADLLA |
| 61 | BAP060-S036K | VVSHFNDCPDSHTQFCFHGTCRFLVQEDKPACVCHKGYVGARCEHADLLA |
| 62 | BAP060-S036N | VVSHFNDCPDSHTQFCFHGTCRFLVQEDKPACVCHNGYVGARCEHADLLA |
| 63 | BAP060-S036R | VVSHFNDCPDSHTQFCFHGTCRFLVQEDKPACVCHRGYVGARCEHADLLA |
| 64 | BAP060-Y038D | VVSHENDCPDSHTQFCFHGTCRFLVQEDKPACVCHSGDVGARCEHADLLA |
| 65 | BAP060-Y038E | VVSHFNDCPDSHTQFCFHGTCRFLVQEDKPACVCHSGEVGARCEHADLLA |
| 66 | BAP060-Y038I | VVSHFNDCPDSHTQFCFHGTCRFLVQEDKPACVCHSGIVGARCEHADLLA |
| 67 | BAP060-Y038M | VVSHFNDCPDSHTQFCFHGTCRFLVQEDKPACVCHSGMVGARCEHADLLA |
| 68 | BAP060-Y038T | VVSHFNDCPDSHTQFCFHGTCRFLVQEDKPACVCHSGTVGARCEHADLLA |
| 69 | BAP060-G040D | VVSHENDCPDSHTQFCFHGTCRFLVQEDKPACVCHSGYVDARCEHADLLA |
| 70 | BAP060-G040E | VVSHFNDCPDSHTQFCFHGTCRFLVQEDKPACVCHSGYVEARCEHADLLA |
| 71 | BAP060-G040K | VVSHFNDCPDSHTQFCFHGTCRFLVQEDKPACVCHSGYVKARCEHADLLA |
| 72 | BAP060-G040Q | VVSHENDCPDSHTQFCFHGTCRFLVQEDKPACVCHSGYVQARCEHADLLA |
| 73 | BAP060-G040S | VVSHENDCPDSHTQFCFHGTCRFLVQEDKPACVCHSGYVSARCEHADLLA |
| 74 | BAP060-A041D | VVSHENDCPDSHTQFCFHGTCRFLVQEDKPACVCHSGYVGDRCEHADLLA |
| 75 | BAP060-A041F | VVSHFNDCPDSHTQFCFHGTCRFLVQEDKPACVCHSGYVGFRCEHADLLA |
| 76 | BAP060-A041H | VVSHFNDCPDSHTQFCFHGTCRFLVQEDKPACVCHSGYVGHRCEHADLLA |
| 77 | BAP060-A041S | VVSHENDCPDSHTQFCFHGTCRFLVQEDKPACVCHSGYVGSRCEHADLLA |
| 78 | BAP060-A041W | VVSHFNDCPDSHTQFCFHGTCRFLVQEDKPACVCHSGYVGWRCEHADLLA |
| 79 | BAP060-A041Y | VVSHFNDCPDSHTQFCFHGTCRFLVQEDKPACVCHSGYVGYRCEHADLLA |
| 80 | BAP060-E044M | VVSHFNDCPDSHTQFCFHGTCRFLVQEDKPACVCHSGYVGARCMHADLLA |
| 81 | BAP060-E044Q | VVSHENDCPDSHTQFCFHGTCRFLVQEDKPACVCHSGYVGARCQHADLLA |
| 82 | BAP060-E044S | VVSHFNDCPDSHTQFCFHGTCRFLVQEDKPACVCHSGYVGARCSHADLLA |
| 83 | BAP060-A046I | VVSHFNDCPDSHTQFCFHGTCRFLVQEDKPACVCHSGYVGARCEHIDLLA |
| 84 | BAP060-A046L | VVSHFNDCPDSHTQFCFHGTCRFLVQEDKPACVCHSGYVGARCEHLDLLA |
| 85 | BAP060-A046V | VVSHFNDCPDSHTQFCFHGTCRFLVQEDKPACVCHSGYVGARCEHVDLLA |
| 86 | BAP060-A046Y | VVSHFNDCPDSHTQFCFHGTCRFLVQEDKPACVCHSGYVGARCEHYDLLA |
| 87 | BAP060-D047N | VVSHFNDCPDSHTQFCFHGTCRFLVQEDKPACVCHSGYVGARCEHANLLA |
| 88 | BAP060-D047W | VVSHFNDCPDSHTQFCFHGTCRFLVQEDKPACVCHSGYVGARCEHAWLLA |
| 89 | BAP060-L048D | VVSHENDCPDSHTQFCFHGTCRFLVQEDKPACVCHSGYVGARCEHADDLA |
| 90 | BAP060-L048K | VVSHFNDCPDSHTQFCFHGTCRFLVQEDKPACVCHSGYVGARCEHADKLA |
| 91 | BAP060-A050F | VVSHFNDCPDSHTQFCFHGTCRFLVQEDKPACVCHSGYVGARCEHADLLF |
| 92 | BAP060-A050H | VVSHFNDCPDSHTQFCFHGTCRFLVQEDKPACVCHSGYVGARCEHADLLH |
| 93 | BAP060-A050K | VVSHFNDCPDSHTQFCFHGTCRFLVQEDKPACVCHSGYVGARCEHADLLK |
| 94 | BAP060-A050R | VVSHFNDCPDSHTQFCFHGTCRFLVQEDKPACVCHSGYVGARCEHADLLR |
| 95 | BAP060-A050W | VVSHFNDCPDSHTQFCFHGTCRFLVQEDKPACVCHSGYVGARCEHADLLW |
| 96 | Q14G-F17L | VVSHFNDCPDSHTGFCLHGTCRFLVQEDKPACVCHSGYVGARCEHADLLA |
| 97 | Q14G-A41D | VVSHENDCPDSHTGFCFHGTCRFLVQEDKPACVCHSGYVGDRCEHADLLA |
| 98 | S36R-A46Y | VVSHFNDCPDSHTQFCFHGTCRFLVQEDKPACVCHRGYVGARCEHYDLLA |
| 99 | E27L-A46Y | VVSHENDCPDSHTQFCFHGTCRFLVQLDKPACVCHSGYVGARCEHYDLLA |
| 100 | F17L-E27L | VVSHENDCPDSHTQFCLHGTCRFLVQLDKPACVCHSGYVGARCEHADLLA |
| 101 | A46Y-A50W | VVSHFNDCPDSHTQFCFHGTCRFLVQEDKPACVCHSGYVGARCEHYDLLW |
| 102 | F17L-A50W | VVSHFNDCPDSHTQFCLHGTCRFLVQEDKPACVCHSGYVGARCEHADLLW |
| 103 | E27L-A50W | VVSHFNDCPDSHTQFCFHGTCRFLVQLDKPACVCHSGYVGARCEHADLLW |
| 104 | Q14G-A46Y-A50W | VVSHFNDCPDSHTGFCFHGTCRFLVQEDKPACVCHSGYVGARCEHYDLLW |
| 105 | Q14G-E27L-S36R | VVSHFNDCPDSHTGFCFHGTCRFLVQLDKPACVCHRGYVGARCEHADLLA |
| 106 | E27L-A41D-A46Y | VVSHFNDCPDSHTQFCFHGTCRFLVQLDKPACVCHSGYVGDRCEHYDLLA |
| 107 | F17L-S36R-A50W | VVSHFNDCPDSHTQFCLHGTCRFLVQEDKPACVCHRGYVGARCEHADLLW |
| 108 | F17L-E27L-A50W | VVSHFNDCPDSHTQFCLHGTCRFLVQLDKPACVCHSGYVGARCEHADLLW |
| 109 | E27L-S36R-A46Y | VVSHENDCPDSHTQFCFHGTCRFLVQLDKPACVCHRGYVGARCEHYDLLA |
| 110 | F17L-S36R-A41D | VVSHFNDCPDSHTQFCLHGTCRFLVQEDKPACVCHRGYVGDRCEHADLLA |
| 111 | Q14G-E27L-A46Y | VVSHENDCPDSHTGFCFHGTCRFLVQLDKPACVCHSGYVGARCEHYDLLA |
| 112 | F17L-S36R-A41D- | VVSHENDCPDSHTQFCLHGTCRFLVQEDKPACVCHRGYVGDRCEHYDLLA |
| | A46Y | |
| 113 | Q14G-F17L-S36R- | VVSHFNDCPDSHTGFCLHGTCRFLVQEDKPACVCHRGYVGARCEHYDLLA |
| | A46Y | |
| 114 | F17L-E27L-A41D- | VVSHENDCPDSHTQFCLHGTCRFLVQLDKPACVCHSGYVGDRCEHYDLLA |
| | A46Y | |
| 115 | F17L-S36R-A46Y- | VVSHFNDCPDSHTQFCLHGTCRFLVQEDKPACVCHRGYVGARCEHYDLLW |
| | A50W | |
| 116 | E27L-S36R-A46Y- | VVSHENDCPDSHTQFCFHGTCRFLVQLDKPACVCHRGYVGARCEHYDLLW |
| | A50W | |
| 117 | E27L-S36R-A41D- | VVSHENDCPDSHTQFCFHGTCRFLVQLDKPACVCHRGYVGDRCEHADLLW |
| | A50W | |
| 118 | F17L-E27L-S36R- | VVSHFNDCPDSHTQFCLHGTCRFLVQLDKPACVCHRGYVGARCEHYDLLA |
| | A46Y | |
| 119 | Q14G-E27L-S36R- | VVSHFNDCPDSHTGFCFHGTCRFLVQLDKPACVCHRGYVGDRCEHADLLA |
| | A41D | |
| 120 | Q14G-A41D-A46Y- | VVSHFNDCPDSHTGFCFHGTCRFLVQEDKPACVCHSGYVGDRCEHYDLLW |
| | A50W | |
| 121 | Q14G-E27L-A41D- | VVSHENDCPDSHTGFCFHGTCRFLVQLDKPACVCHSGYVGDRCEHYDLLA |
| | A46Y | |
| 122 | Q14G-S36R-A46Y- | VVSHFNDCPDSHTGFCFHGTCRFLVQEDKPACVCHRGYVGARCEHYDLLW |
| | A50W | |
| 123 | Q14G-F17L-E27L- | VVSHENDCPDSHTGFCLHGTCRFLVQLDKPACVCHSGYVGARCEHYDLLA |
| | A46Y | |
| 124 | Q14G-F17L-A46Y- | VVSHFNDCPDSHTGFCLHGTCRFLVQEDKPACVCHSGYVGARCEHYDLLW |
| | A50W | |
| 125 | Q14G-F17L-E27L- | VVSHFNDCPDSHTGFCLHGTCRFLVQLDKPACVCHSGYVGDRCEHADLLA |
| | A41D | |
| 126 | F17L-E27L-A46Y- | VVSHFNDCPDSHTQFCLHGTCRFLVQLDKPACVCHSGYVGARCEHYDLLW |
| | A50W | |
| 127 | Q14G-F17L-E27L- | VVSHFNDCPDSHTGFCLHGTCRFLVQLDKPACVCHSGYVGARCEHADLLW |
| | A50W | |
| 128 | Q14G-F17L-E27L- | VVSHENDCPDSHTGFCLHGTCRFLVQLDKPACVCHRGYVGARCEHYDLLA |
| | S36R-A46Y | |
| 129 | Q14G-F17L-A41D- | VVSHFNDCPDSHTGFCLHGTCRFLVQEDKPACVCHSGYVGDRCEHYDLLW |
| | A46Y-A50W | |
| 130 | Q14G-S36R-A41D- | VVSHFNDCPDSHTGFCFHGTCRFLVQEDKPACVCHRGYVGDRCEHYDLLW |
| | A46Y-A50W | |
| 131 | Q14G-E27L-S36R- | VVSHFNDCPDSHTGFCFHGTCRFLVQLDKPACVCHRGYVGDRCEHADLLW |
| | A41D-A50W | |
| 132 | F17L-E27L-S36R- | VVSHFNDCPDSHTQFCLHGTCRFLVQLDKPACVCHRGYVGDRCEHYDLLA |
| | A41D-A46Y | |
| 133 | F17L-S36R-A41D- | VVSHFNDCPDSHTQFCLHGTCRFLVQEDKPACVCHRGYVGDRCEHYDLLW |
| | A46Y-A50W | |
| 134 | Q14G-F17L-S36R- | VVSHFNDCPDSHTGFCLHGTCRFLVQEDKPACVCHRGYVGDRCEHADLLW |
| | A41D-A50W | |
| 135 | Q14G-F17L-E27L- | VVSHFNDCPDSHTGFCLHGTCRFLVQLDKPACVCHSGYVGDRCEHADLLW |
| | A41D-A50W | |
| 136 | E27L-S36R-A41D- | VVSHFNDCPDSHTQFCFHGTCRFLVQLDKPACVCHRGYVGDRCEHYDLLW |
| | A46Y-A50W | |
| 137 | Q14G-F17L-E27L- | VVSHENDCPDSHTGFCLHGTCRFLVQLDKPACVCHSGYVGARCEHYDLLW |
| | A46Y-A50W | |
| 138 | Q14G-F17L-E27L- | VVSHFNDCPDSHTGFCLHGTCRFLVQLDKPACVCHSGYVGDRCEHYDLLA |
| | A41D-A46Y | |
| 139 | F17L-E27L-S36R- | VVSHENDCPDSHTQFCLHGTCRFLVQLDKPACVCHRGYVGDRCEHADLLW |
| | A41D-A50W | |
| 140 | F17L-E27L-S36R- | VVSHFNDCPDSHTQFCLHGTCRFLVQLDKPACVCHRGYVGDRCEHYDLLW |
| | A41D-A46Y-A50W | |
| 141 | Q14G-F17L-E27L- | VVSHFNDCPDSHTGFCLHGTCRFLVQLDKPACVCHSGYVGDRCEHYDLLW |
| | A41D-A46Y-A50W | |
| 142 | Q14G-F17L-E27L- | VVSHFNDCPDSHTGFCLHGTCRFLVQLDKPACVCHRGYVGDRCEHADLLW |
| | S36R-A41D-A50W | |
| 143 | Q14G-F17L-E27L- | VVSHFNDCPDSHTGFCLHGTCRFLVQLDKPACVCHRGYVGDRCEHYDLLW |
| | S36R-A41D-A46Y- | |
| | A50W | |

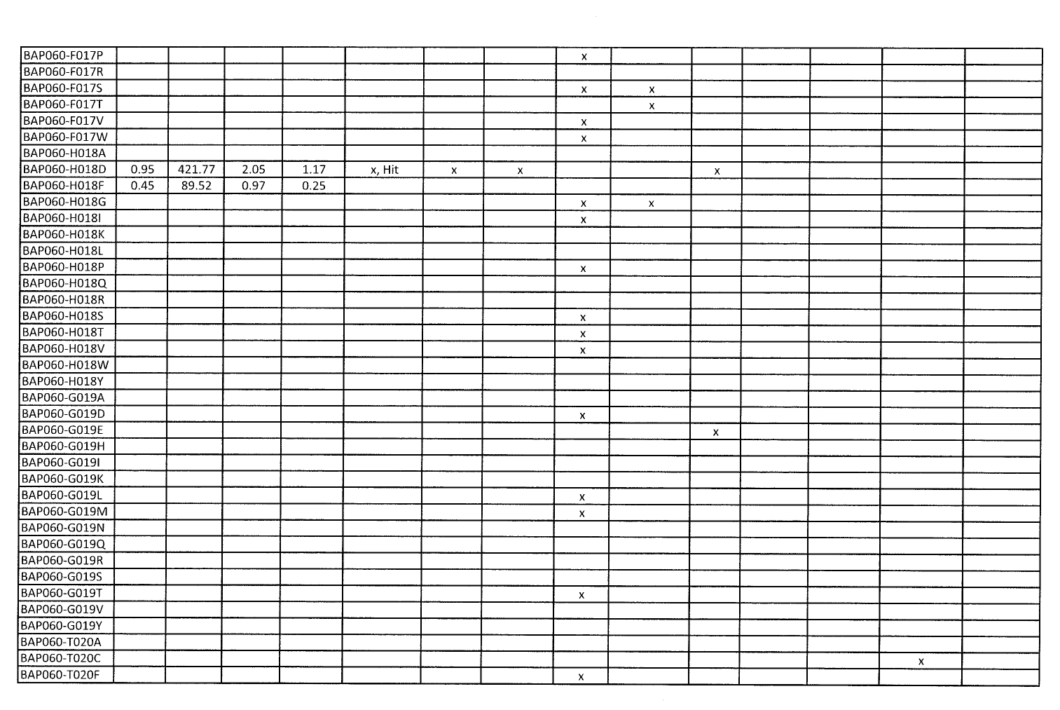

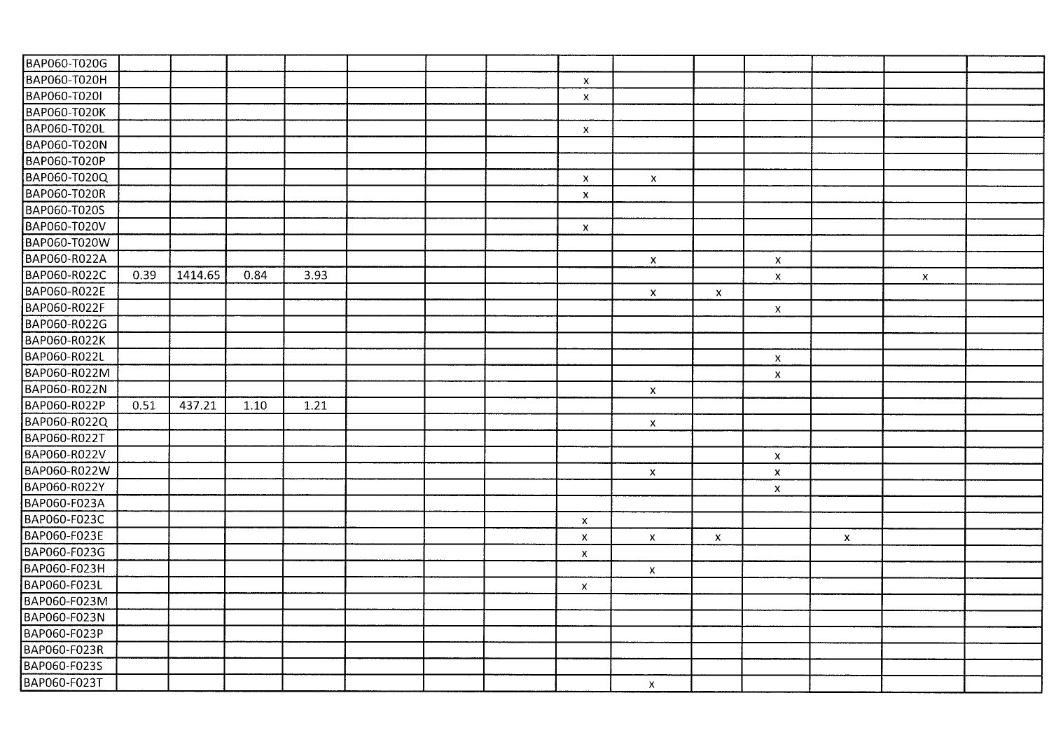

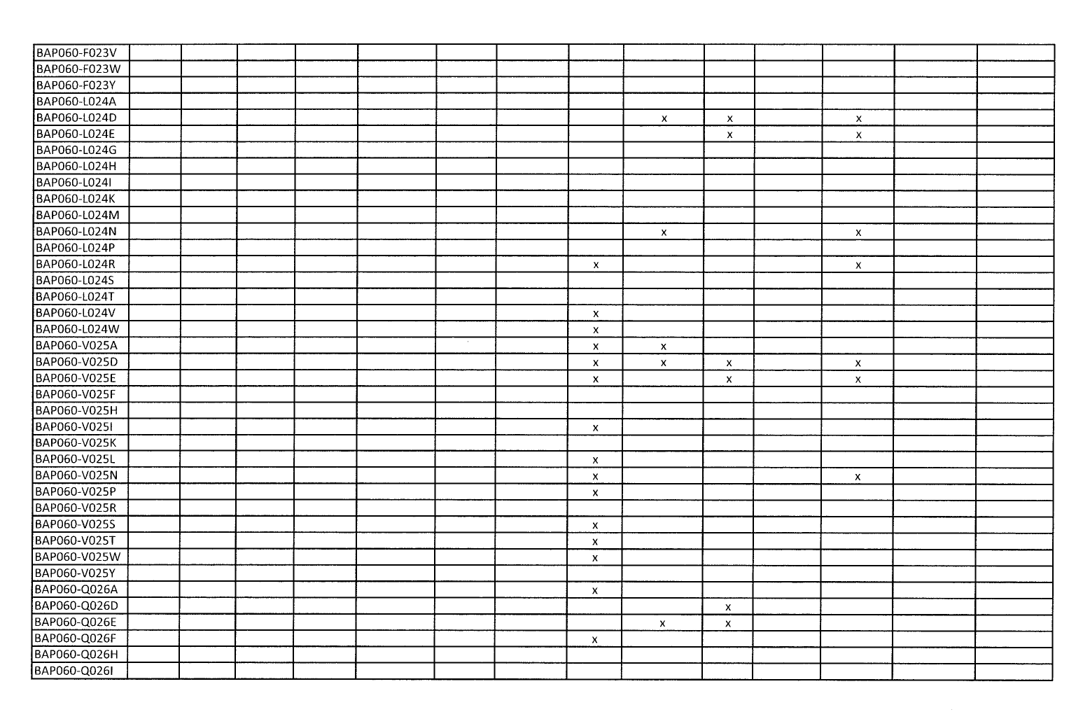

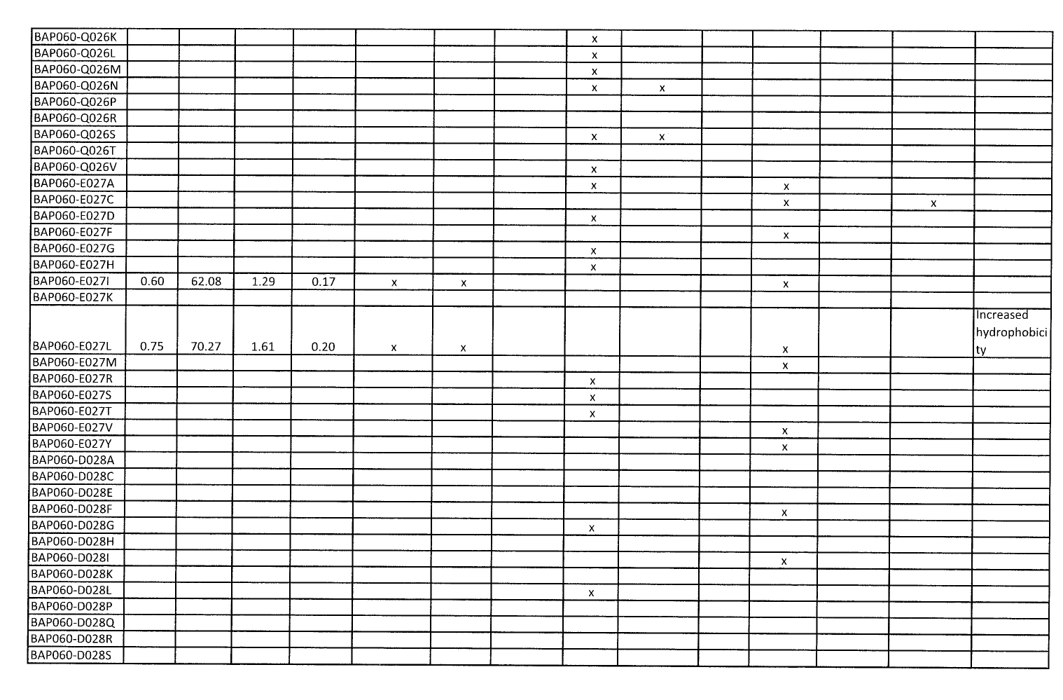

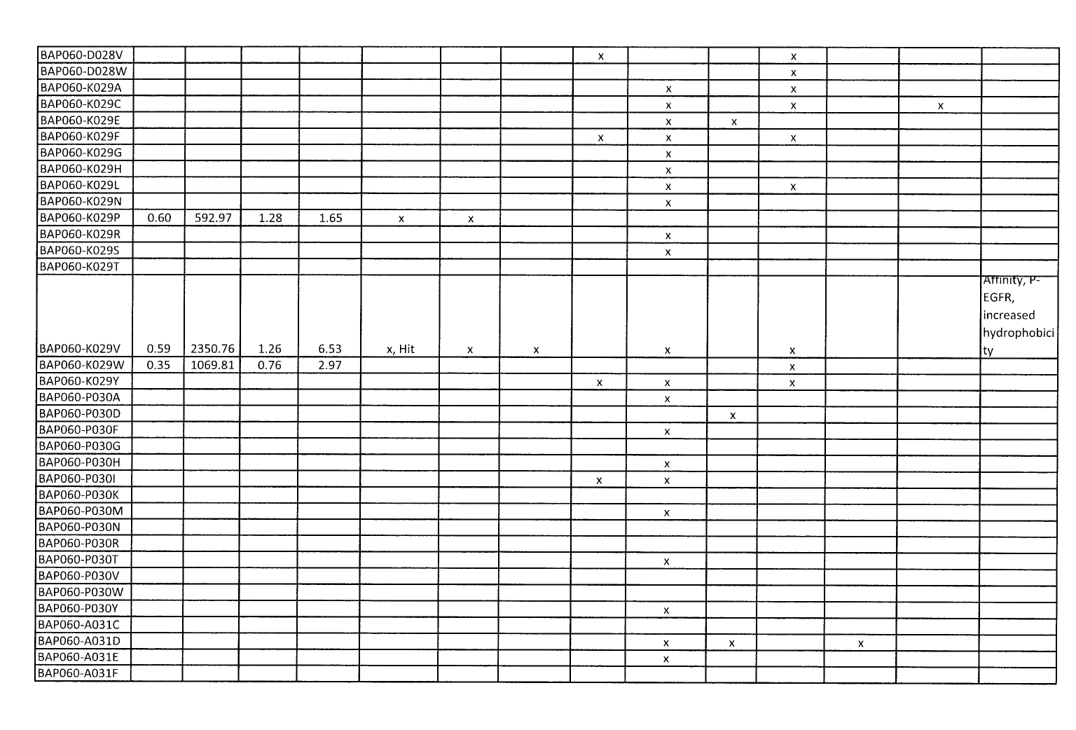

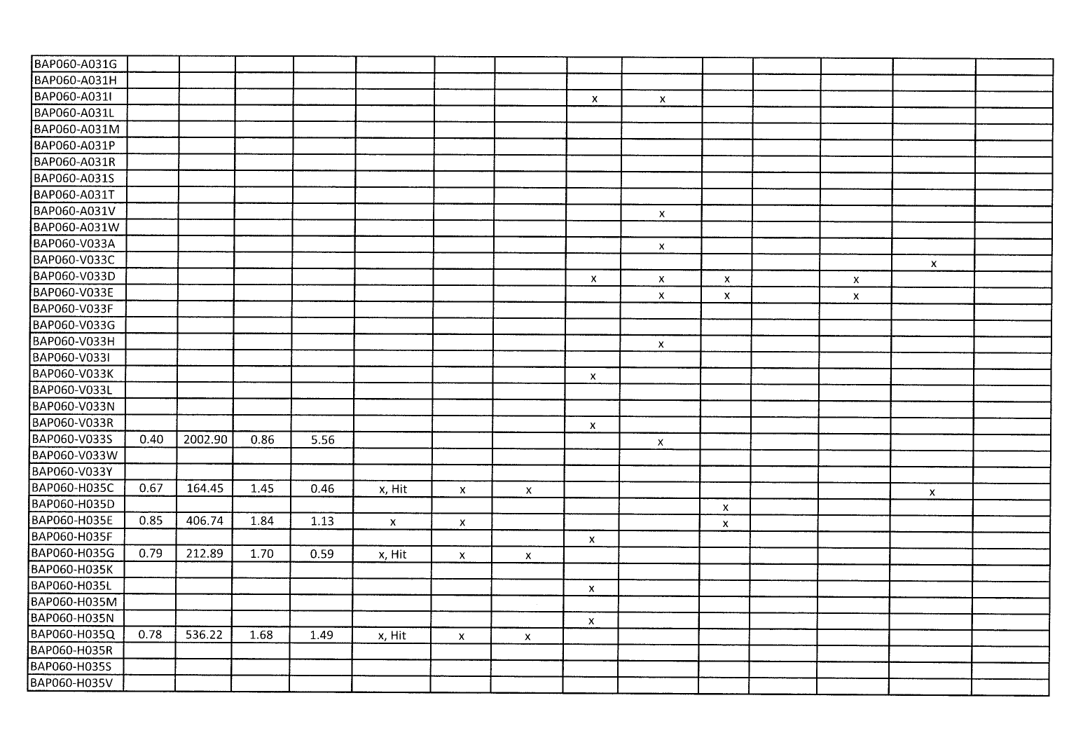

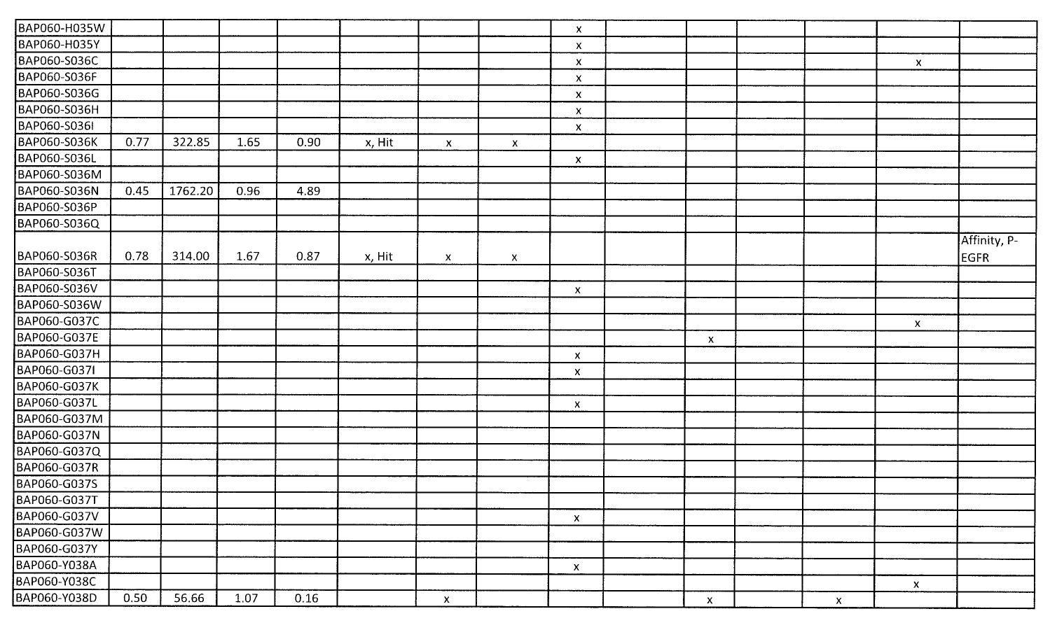

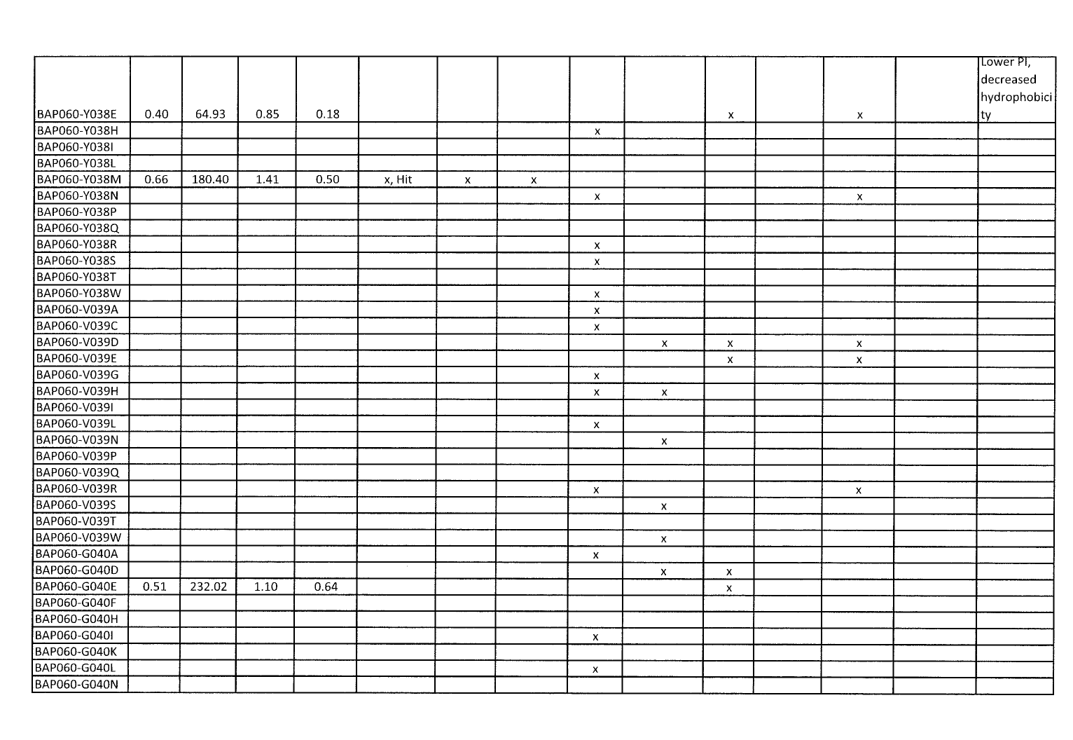

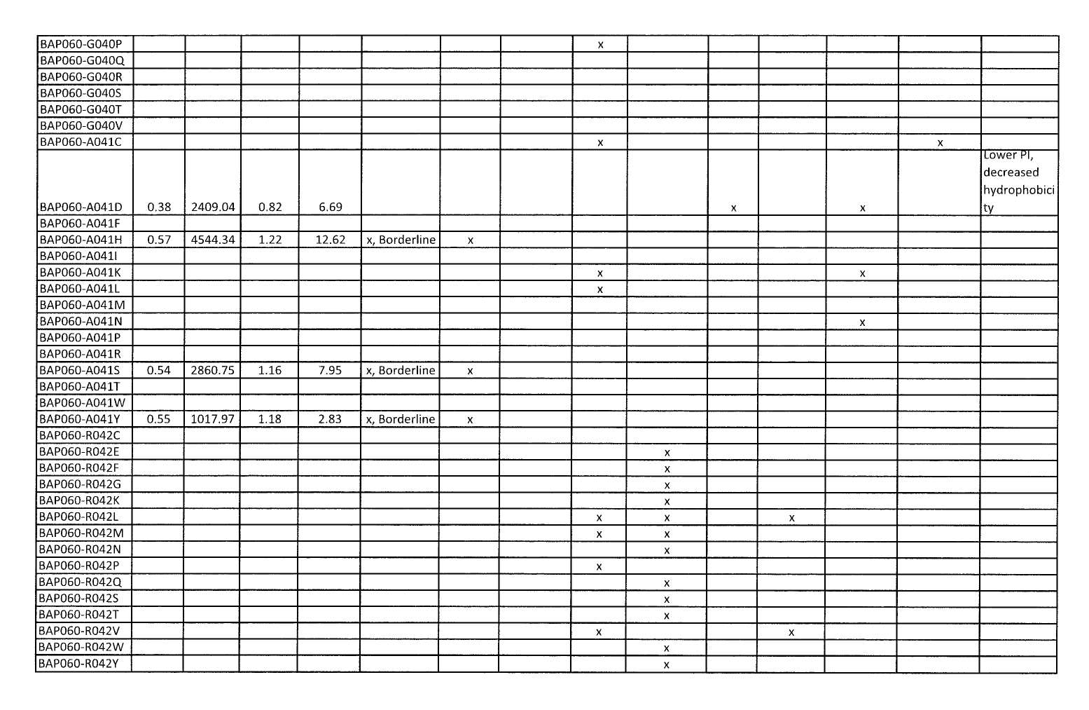

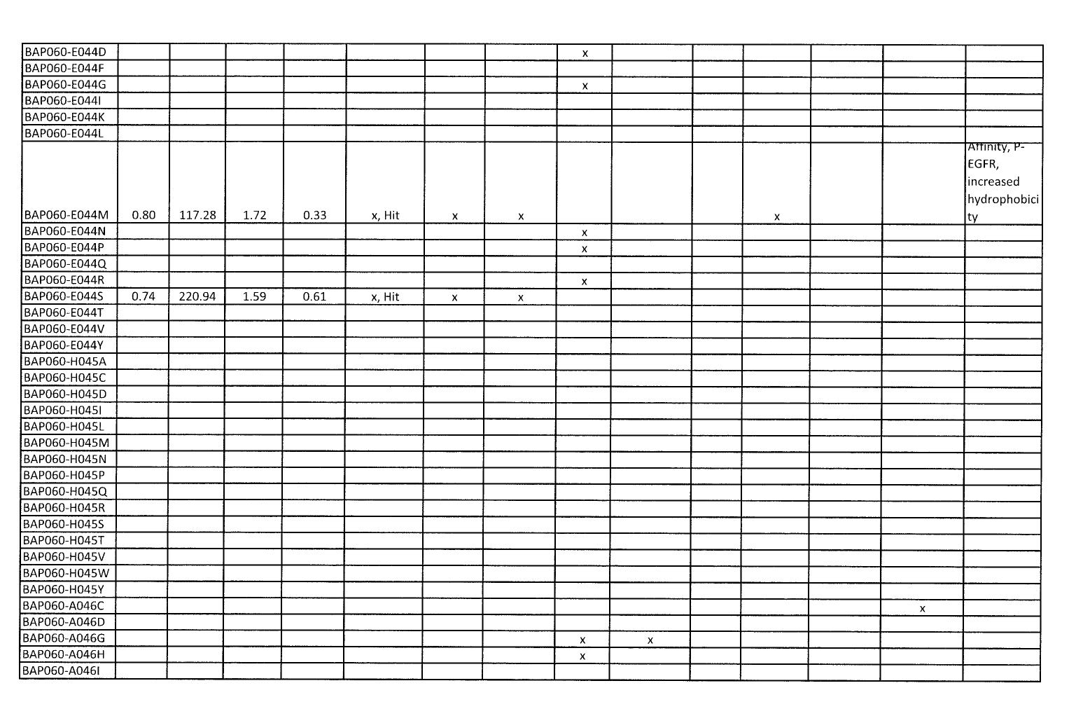

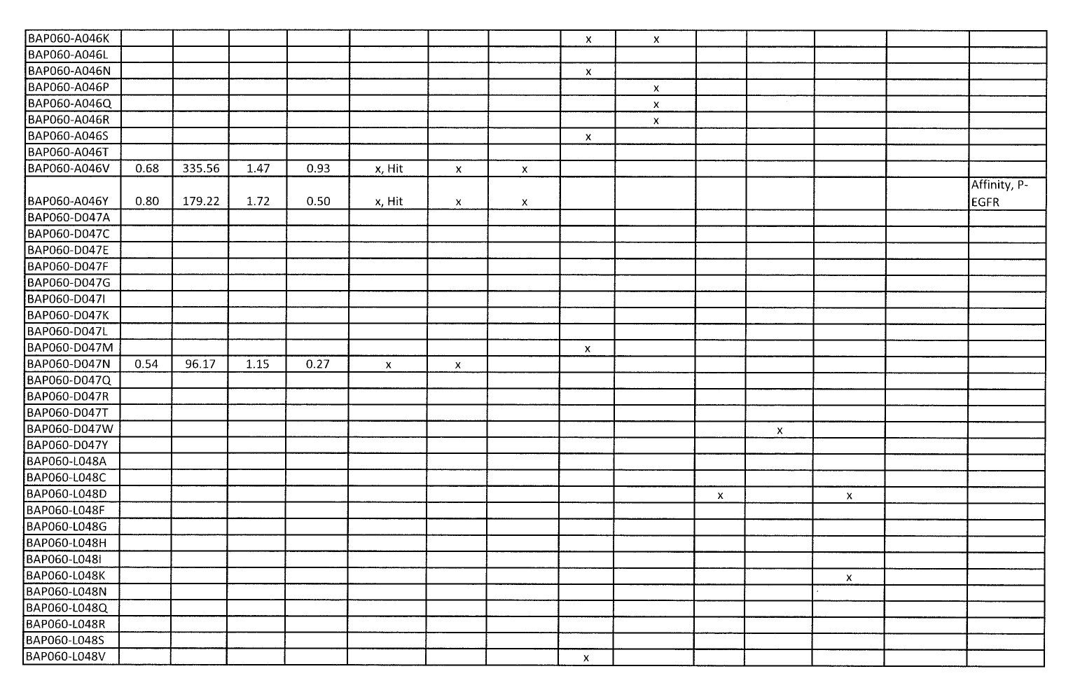

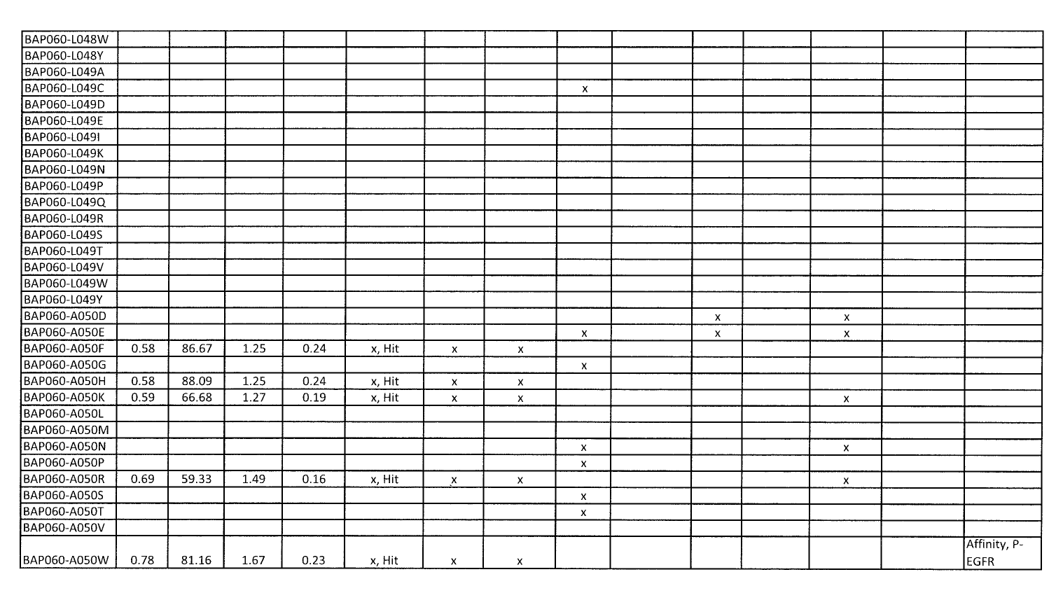

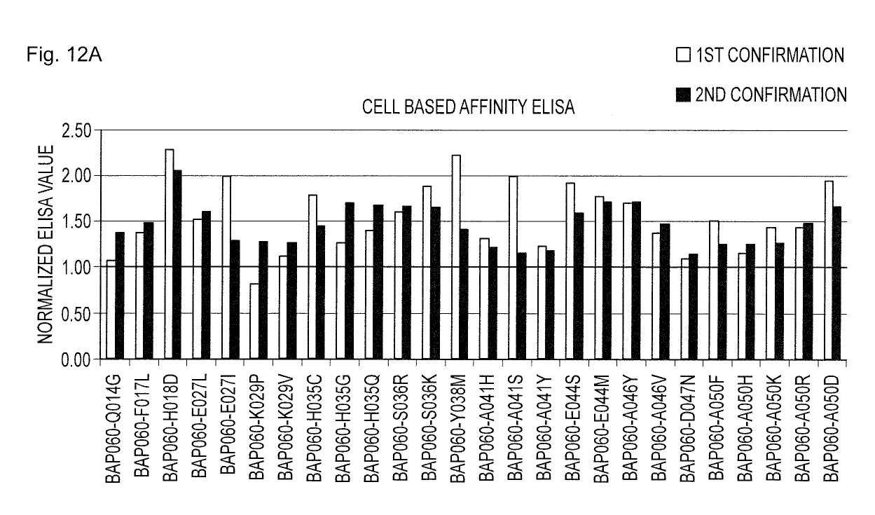

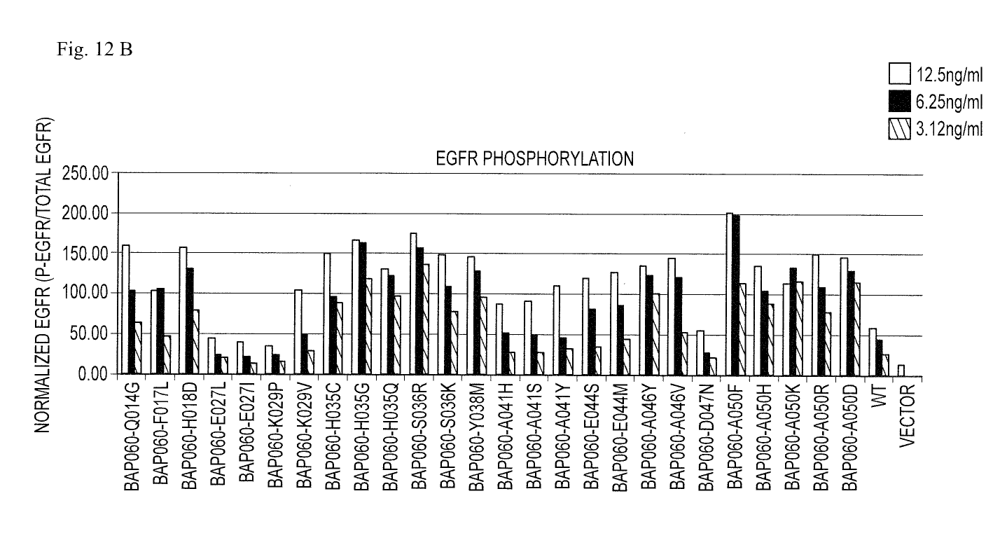

A first confirmation was performed by re-transforming E. coli BL21 cells with plasmids containing the 94 CPE™ mutants, collected the periplasmic fractions after IPTG induction and performed quantitation ELISA and cell based ELISA. The results of the first confirmation are shown in FIG. 11A. This first confirmation identified 37 CPE™ mutants having a higher binding activity compared to the wild type TGFα . These were tested for 2^{nd} confirmation, the results of which are shown in FIG. 11B and FIG. 12A. The second confirmation identified 27 CPE™ putative hits that consistently had higher binding activity compared to the wild type TGFα. These 27 CPE™ mutants were then tested in a cell based EGFR phosphorylation assay to confirm the functional activity of the CPE™ mutants. (FIGS. 11B and 12B)

EGFR phosphorylation. BAP060-hTGFα -B (mature hTGFα, aa 40-89, with PE1B signal sequence) was transformed into E. coli BL21 (DE3) competent cells. Protein was expressed upon the addition of 1 mM IPTG at 37° C. for 3 hours in 14 ml tubes. Vector only transformed BL21 cultures were also included. Cells were collected after induction. Periplasmic proteins were prepared by incubating the cells in OS buffer (200 mM Tris-HCl, pH 7.5, 20% sucrose, 1 mM EDTA) with DNAse, RNAse and lysozyme. After addition of lysis buffer (10 mM Tris, pH 7.5, 50 mM KCl, 1 mM EDTA and 0.1% of Na deoxycholate) to the cells in OS buffer, the cells were centrifuged. The supernatants, which contain the periplasmic fractions, were transferred to another microcentrifuge tube. A431 cells were seeded in 96 well plates and serum starved overnight. The cells were washed in serum free media and incubated with 50 ul of hTGFα starting 12.5 ng/ml , 6.25 ng/ml or 3.125 ng/ml at 37° C. for 10 min: The cells were then washed with PBS and lysed. Half of the cell lysate were used to determine the level of total EGFR using Cell Signaling kit #7250 and the other half of the cell lysate were used to determined the level of phosphorylated EGFR using Cell Signaling kit #7240 following vendor's protocols. The results are shown in FIG. 12B.

The above referenced methodology identified 19 CPE™ mutants having a higher binding activity in cell based ELISA, and an increased ability to induce the phosphorylation of EGFR in the cell based EGFR phosphorylation assay, compared to the wild type TGFα (FIG. 11B, FIGS. 12A & B).

### hTGFα Evolution with CPS™

1. Construction of the CPS™ Library of Human TGFα

7 CPE™ mutants were selected based on their superior or different characteristics compared to the wild type hTGFα: binding activity to hTGFα, ability to induce EGFR phosphorylation in A431 cells, hydrophobicity, soluble expression in E. coli and isoelectric points. The locations and nature of the mutations in these mutants are shown in Table 3.

TABLE 3
List of CPE ™ Mutants for CPS ™ Process

| CPE ™ Clone | Mutant | AA | Mutant | |
|---|---|---|---|---|
| Name | Codon | Position | AA | Mutant Characteristics |
| BAP060-Q014G | GGT | 014 | G | Increased affinity, |
| | | | | P-EGFR induction |
| BAP060-F017L | TTG | 017 | L | Increased affinity, |
| | | | | P-EGFR induction, |
| | | | | periplasmic expression |
| BAP060-E027L | CTG | 027 | L | Neutral affinity, |
| | | | | P-EGFR induction |
| BAP060-S036R | CGG | 036 | R | Increased affinity, |
| | | | | P-EGFR induction |
| BAP060-A041D | GAT | 041 | D | Neutral affinity, |
| | | | | lower PI, decreased |
| | | | | hydrophobicity |
| BAP060-A046Y | TAT | 046 | Y | Increased affinity, |
| | | | | P-EGFR induction |
| BAP060-A050W | TGG | 050 | W | Increased affinity, |
| | | | | P-EGFR induction |

A Combinatorial Protein Synthesis (CPS™) library of BAP060-hTGFα-B mutants containing all possible combinations of mutations in the selected 7 CPE™ mutants was made. The resulting 49 CPS™ mutant hTGFα proteins were sequenced and arrayed in 96-well format. The sequences of these new mutants are shown in Table 2 and FIG. 16.

2. Expression of BAP060-hTGFα -B CPS™ Mutants in E. coli BL21 (DE3) Cells and Determination of Binding Activity

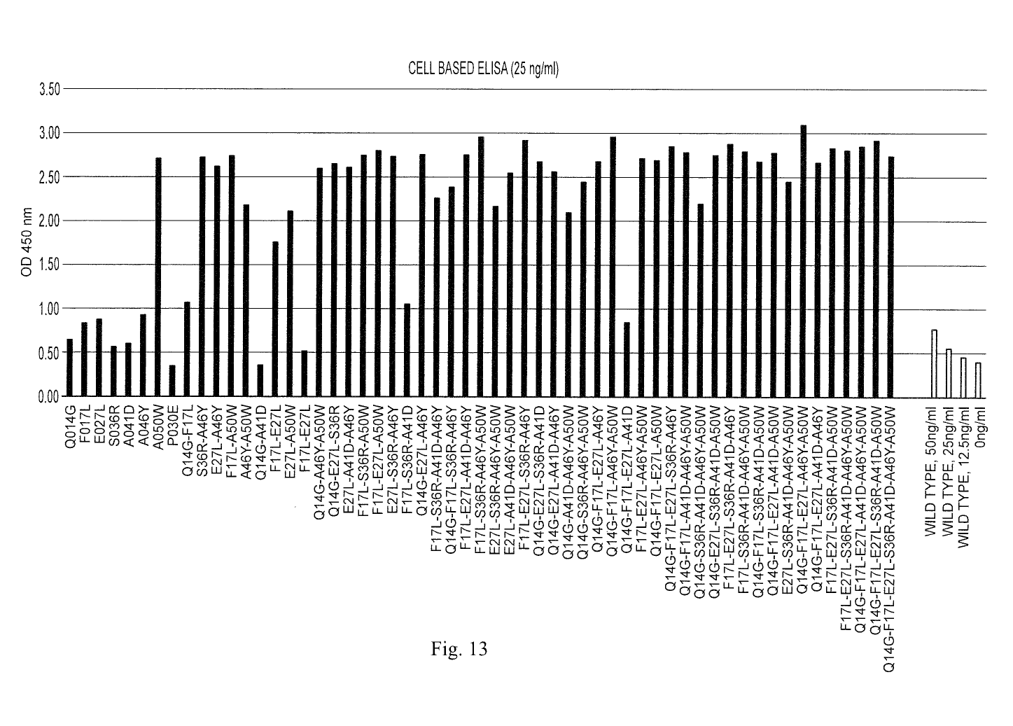

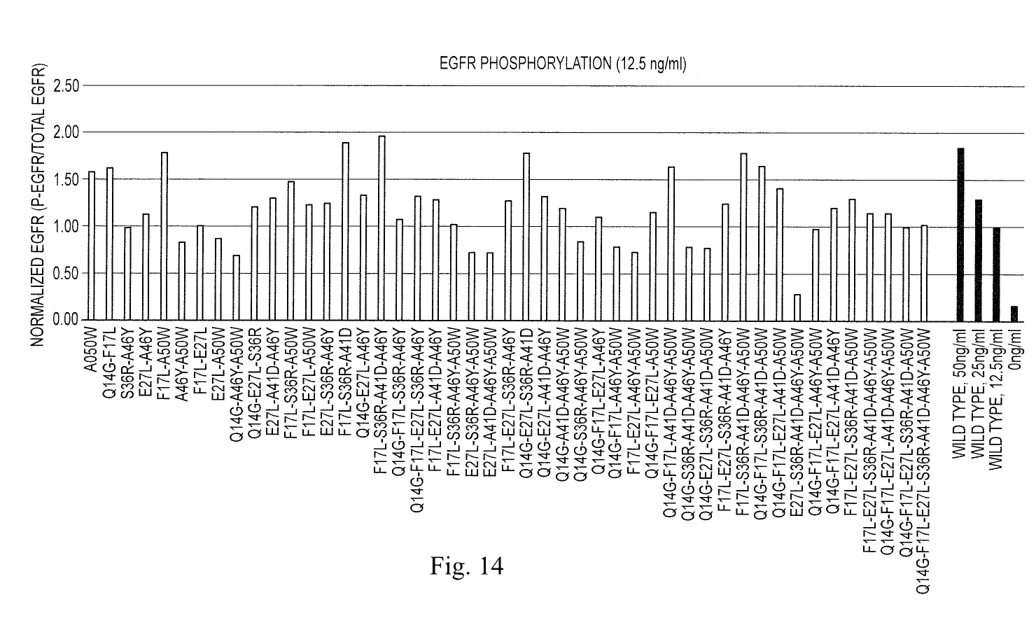

E. coli BL21 cells were transformed with BAP060-hTGFα -B CPS™ mutants and induced with IPTG at 37° C. The periplasmic fractions after IPTG induction were collected and the expression levels of hTGFα determined by quantitation ELISA, as described above. From this, 46 CPS™ mutants (containing up to 7 mutations) were identified as having at least 2 fold higher binding activity compared to the wild type hTGFα, and also higher binding activity than most of the CPE™ mutants (FIG. 13). These 46 CPS™ mutants were also tested by cell based EGFR phosphorylation assay to determine their functional activities. Briefly, BAP060-hTGFα-B (mature hTGFα, aa 40-89, with PE1B signal sequence) or CPS™ mutants was transformed into E. coli BL21 (DE3) competent cells in 96 well format. Protein was expressed upon the addition of 1 mM IPTG at 37° C. for 3 hours in 14 ml tubes. Vector only transformed BL21 cultures were also included. Cells were collected after induction, and periplasmic proteins prepared by incubating the cells in OS buffer with DNAse, RNAse and lysozyme. After addition of lysis buffer to the cells in OS buffer, the cells were centrifuged, and the supernatants containing the periplasmic fractions transferred to a clean set of microcentrifuge tubes. A431 cells were seeded in 96 well plates and serum starved overnight. The cells were washed in serum free media and incubated with 50 ul of wild type hTGFα starting at 50 ng/ml, 25 ng/ml and 12.5 ng/ml (black bars) or 12.5 ng/ml CPS™ mutants (white bars) at 37° C. for 10 min: The cells were then washed with PBS and lysed. Half of the cell lysate were used to determine the level of total EGFR using Cell Signaling kit #7250 and the other half of the cell lysate were used to determine the level of phosphorylated EGFR using Cell Signaling kit #7240 following vendor's protocols. The results RGFR phosphorylation results are shown in FIG. 14.

In addition, 15 CPS™ mutants (containing up to 5 mutations) having an increased ability to induce the phosphorylation of EGFR, relative to the wild type hTGFα (FIG. 14), were selected for cell based titration ELISA to confirm their binding activities (FIG. 15). Briefly, BAP060-hTGFα-B (mature hTGFα, aa 40-89, with PelB signal sequence) wild type and CPS™ mutants were transformed into E. coli BL21 (DE3) competent cells. Protein was expressed upon the addition of 1 mM IPTG at 37° C. for 3 hours in 96 well format. Cells were collected after induction and periplasmic proteins prepared by incubating the cells in OS buffer with DNAse, RNAse and lysozyme. Lysis buffer was added to the cells in OS buffer, the cells centrifuged, and the supernatants containing the periplasmic fractions transferred to a clean set of microcentrifuge tubes. Concentration of hTGFα in each sample was determined by quantitation ELISA. 3.125 ng/ml, 1.56 ng/ml, 0.78 ng/ml, or 0.34 ng/ml of wild type hTGFα or CPS™ mutant hTGFα were incubated with A431 cells (ATCC#CRL1555) immobilized on the plate. In addition, 100 ng/ml, 50 ng/ml, 25 ng/ml, 12.5 ng/ml, 6.25 ng/ml, 3.125 ng/ml, 1.56 ng/ml, or 0.78 ng/ml of wild type hTGFα were included as control. Bound hTGFα was detected with biotinylated anti-hTGFα (R&D Systems, #BAF239) and Streptavidin conjugated with HRP. The reactions were stopped by addition of HCl after TMB was added to the wells and read immediately. OD450 nm value of the reactions was measured with a Molecular Device SPECTRAmax Plus plate reader. The results of this assay are shown in FIG. 15.

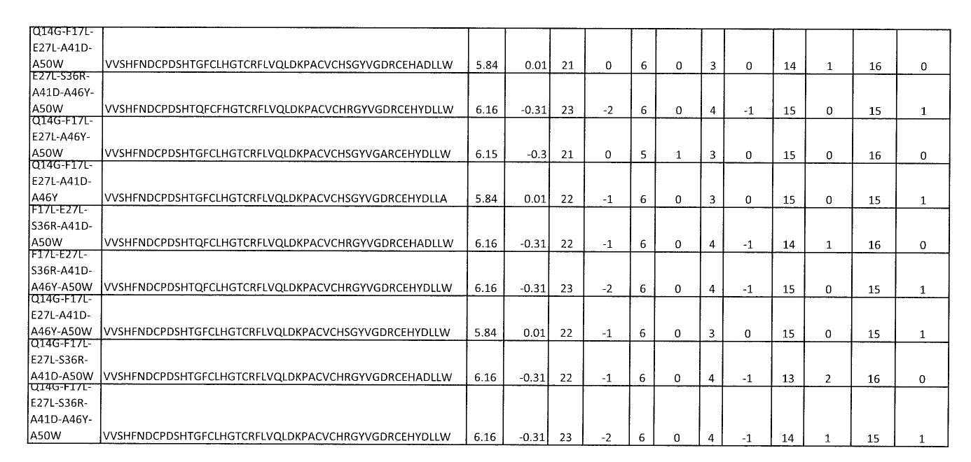

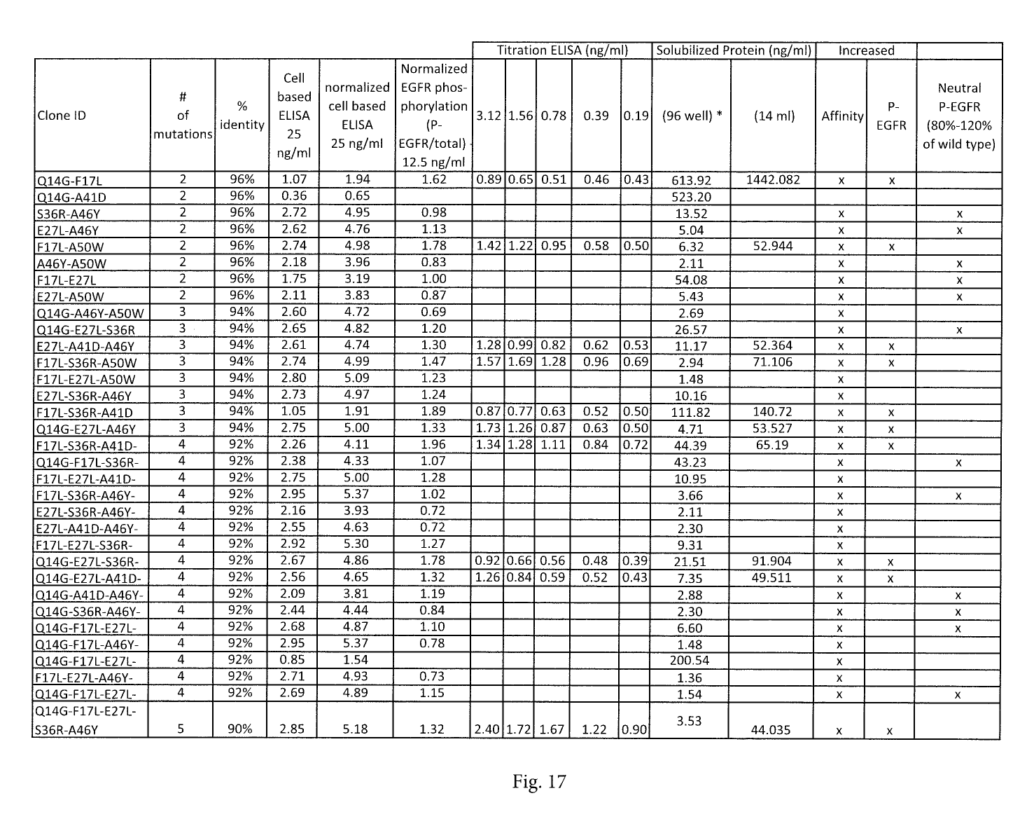

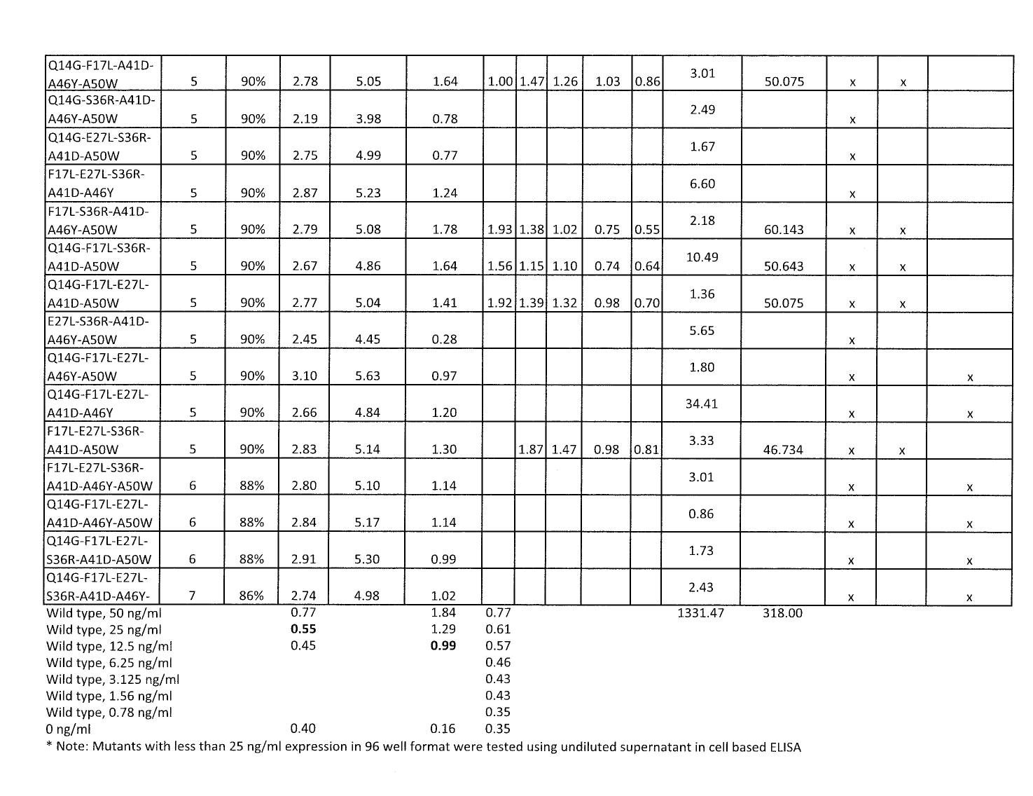

In summary, the methodology disclosed above yielded hTGFα mutant proteins having different physical characteristics, such as hydrophobicity and isoelectric points, compared to the wild type hTGFα (FIG. 16). The methodology also yielded hTGFα mutant proteins that performed as well as, or better than, the wild type hTGFα in binding to cell-surface EGFR, and in inducing the phosphorylation of EGFR in cells (FIG. 17).

1-10. (canceled)

11. A transforming growth factor-alpha (TGF-α ) protein variant comprising one or more amino acid substitutions, wherein the amino acid residues being substituted correspond to amino acids at one or more positions selected from the group consisting of amino acid position 14, 17, 18, 27, 29, 35, 36, 38, 41, 44, 46, 47, and 50 in a wild-type TGF-α protein.

12. The TGF-α , protein variant of claim 11, wherein the substitutions are conservative substitutions.

13. The TGF-α protein variant of claim 11, wherein the wild-type TGF-α protein comprises SEQ ID NO:1

14. The TGF-α protein variant of claim 11, wherein the protein variant comprises an amino acid sequence at least 85% identical to a wild-type TGF-α protein.

15. The TGF-α protein variant of claim 11, wherein the substitutions are selected from the group consisting of:
a) substitution of the amino acid residue corresponding to the amino acid at position 14 of a wild-type TGF-α protein, with an amino acid selected from the group consisting of aspartic acid, glycine, isoleucine, and phenylalanine;
b) substitution of the amino acid residue corresponding to the amino acid at position 17 of a wild-type TGF-α protein, with an amino acid selected from the group consisting of isoleucine and leucine;
c) substitution of the amino acid residue corresponding to the amino acid at position 18 of a wild-type TGF-α protein, with an amino acid selected from the group consisting of aspartic acid and phenylalanine;
d) substitution of the amino acid residue corresponding to the amino acid at position 27 of a wild-type TGF-α protein, with an amino acid selected from the group consisting of leucine, isoleucine, and phenylalanine;
e) substitution of the amino acid residue corresponding to the amino acid at position 29 of a wild-type TGF-α protein, with an amino acid selected from the group consisting of aspartic acid, glycine, leucine, asparagine, proline, valine, and tryptophan;
f) substitution of the amino acid residue corresponding to the amino acid at position 35 of a wild-type TGF-α protein, with an amino acid selected from the group consisting of cysteine, aspartic acid, glutamic acid, glycine, and glutamine;
g) substitution of the amino acid residue corresponding to the amino acid at position 36 of a wild-type TGF-α protein, with an amino acid selected from the group consisting of lysine, asparagine, and arginine;
h) substitution of the amino acid residue corresponding to the amino acid at position 38 of a wild-type TGF-α protein, with an amino acid selected from the group consisting of aspartic acid, glutamic acid, isoleucine, methionine, and threonine;
i) substitution of the amino acid residue corresponding to the amino acid at position 41 of a wild-type TGF-α protein, with an amino acid selected from the group consisting of aspartic acid, phenylalanine, histidine, serine, and tryptophan;
j) substitution of the amino acid residue corresponding to the amino acid at position 44 of a wild-type TGF-α protein, with an amino acid selected from the group consisting of methionine, glutamine, and serine;
k) substitution of the amino acid residue corresponding to the amino acid at position 46 of a wild-type TGF-α protein, with an amino acid selected from the group consisting of isoleucine, leucine, valine, and tyrosine;
I) substitution of the amino acid residue corresponding to the amino acid at position 47 of a wild-type TGF-α protein, with an amino acid selected from the group consisting of asparagine and tryptophan; and, m) substitution of the amino acid residue corresponding to the amino acid at position 50 of a wild-type TGF-α protein, with an amino acid selected from the group consisting of phenylalanine, histidine, lysine, arginine, and tryptophan.

16. The TGF-α protein variant of claim 15, wherein the sequence of the protein variant is at least 85% identical to a wild-type TGF-α protein.

17. The TGF-α protein variant of claim 16, wherein the wild-type TGF-α protein comprises SEQ ID NO:1.

18. The TGF-α protein variant of claim 11, wherein the protein variant comprises an amino acid sequence selected from the group consisting of SEQ ID NO:2 SEQ ID NO:143.

19. A pharmaceutical composition comprising the TGF-α protein variant of claim 11.

20. A kit comprising the TGF-α protein variant of claim 11.

21. A nucleic acid molecule encoding the TGF-α, protein variant of claim 11.

22. A cell comprising the nucleic acid molecule of claim 21.

23. A pharmaceutical composition comprising the nucleic acid molecule of claim 21.

24. A method for treating an individual having a neurological deficit, the method comprising administering to the individual a transforming growth factor-alpha (TGF-α) protein variant comprising one or more amino acid substitutions, wherein the amino acid residues being substituted correspond to amino acids at one or more positions selected from the group consisting of amino acid position 14, 17, 18, 27, 29, 35, 36, 38, 41, 44, 46, 47, and 50 in a wild-type TGF-α protein.

25. The method of claim 24, wherein the wild-type TGF-α protein comprises SEQ ID NO:1.

26. The method of claim 24, wherein the substitutions are conservative substitutions.

27. The method of claim 24, wherein the TGF-α protein variant comprises an amino acid sequence at least 85% identical to a wild-type TGF-α protein.

28. A method for treating an individual having a neurological deficit, the method comprising administering to the individual a nucleic acid molecule encoding a transforming growth factor-alpha (TGF-α) protein variant comprising one or more amino acid substitutions, wherein the amino acid residues being substituted correspond to amino acids at one or more positions selected from the group consisting of amino acid position 14, 17, 18, 27, 29, 35, 36, 38, 41, 44, 46, 47, and 50 in a wild-type TGF-α protein.

29. The method of claim 28, wherein the wild-type TGF-α protein comprises SEQ Ill NO:1.

30. The method of claim 28, wherein the substitutions are conservative substitutions.


SEQUENCE LISTING (149 sequences)

SEQ ID NO: 1 - protein, 50 aa, Homo sapiens
VVSHFNDCPDSHTQFCFHGTCRFLVQEDKPACVCHSGYVGARCEHADLLA

SEQ ID NO: 2 - protein, 50 aa, Artificial Sequence
  BAP060-V002W
VWSHFNDCPDSHTQFCFHGTCRFLVQEDKPACVCHSGYVGARCEHADLLA

SEQ ID NO: 3 - protein, 50 aa, Artificial Sequence
  BAP060-F005G
VVSHGNDCPDSHTQFCFHGTCRFLVQEDKPACVCHSGYVGARCEHADLLA

SEQ ID NO: 4 - protein, 50 aa, Artificial Sequence
  BAP060-F005K
VVSHKNDCPDSHTQFCFHGTCRFLVQEDKPACVCHSGYVGARCEHADLLA

SEQ ID NO: 5 - protein, 50 aa, Artificial Sequence
  BAP060-F005S
VVSHSNDCPDSHTQFCFHGTCRFLVQEDKPACVCHSGYVGARCEHADLLA

SEQ ID NO: 6 - protein, 50 aa, Artificial Sequence
  BAP060-N006S
VVSHFSDCPDSHTQFCFHGTCRFLVQEDKPACVCHSGYVGARCEHADLLA

SEQ ID NO: 7 - protein, 50 aa, Artificial Sequence
  BAP060-D007H
VVSHFNHCPDSHTQFCFHGTCRFLVQEDKPACVCHSGYVGARCEHADLLA

SEQ ID NO: 8 - protein, 50 aa, Artificial Sequence
  BAP060-Q014D
VVSHFNDCPDSHTDFCFHGTCRFLVQEDKPACVCHSGYVGARCEHADLLA

SEQ ID NO: 9 - protein, 50 aa, Artificial Sequence
  BAP060-Q014G
VVSHFNDCPDSHTGFCFHGTCRFLVQEDKPACVCHSGYVGARCEHADLLA

SEQ ID NO: 10 - protein, 50 aa, Artificial Sequence
  BAP060-Q014I
VVSHFNDCPDSHTIFCFHGTCRFLVQEDKPACVCHSGYVGARCEHADLLA

SEQ ID NO: 11 - protein, 50 aa, Artificial Sequence
  BAP060-Q014P
VVSHFNDCPDSHTPFCFHGTCRFLVQEDKPACVCHSGYVGARCEHADLLA

SEQ ID NO: 12 - protein, 50 aa, Artificial Sequence
  BAP060-F017I
VVSHFNDCPDSHTQFCIHGTCRFLVQEDKPACVCHSGYVGARCEHADLLA

SEQ ID NO: 13 - protein, 50 aa, Artificial Sequence
  BAP060-F017L
VVSHFNDCPDSHTQFCLHGTCRFLVQEDKPACVCHSGYVGARCEHADLLA

SEQ ID NO: 14 - protein, 50 aa, Artificial Sequence
  BAP060-H018D
VVSHFNDCPDSHTQFCFDGTCRFLVQEDKPACVCHSGYVGARCEHADLLA

SEQ ID NO: 15 - protein, 50 aa, Artificial Sequence
  BAP060-H018F
VVSHFNDCPDSHTQFCFFGTCRFLVQEDKPACVCHSGYVGARCEHADLLA

SEQ ID NO: 16 - protein, 50 aa, Artificial Sequence
  BAP060-G019E
VVSHFNDCPDSHTQFCFHETCRFLVQEDKPACVCHSGYVGARCEHADLLA

SEQ ID NO: 17 - protein, 50 aa, Artificial Sequence
  BAP060-T020P
VVSHFNDCPDSHTQFCFHGPCRFLVQEDKPACVCHSGYVGARCEHADLLA

SEQ ID NO: 18 - protein, 50 aa, Artificial Sequence
  BAP060-R022A
VVSHFNDCPDSHTQFCFHGTCAFLVQEDKPACVCHSGYVGARCEHADLLA

SEQ ID NO: 19 - protein, 50 aa, Artificial Sequence
  BAP060-R022C
VVSHFNDCPDSHTQFCFHGTCCFLVQEDKPACVCHSGYVGARCEHADLLA

SEQ ID NO: 20 - protein, 50 aa, Artificial Sequence
  BAP060- R022E
VVSHFNDCPDSHTQFCFHGTCEFLVQEDKPACVCHSGYVGARCEHADLLA

SEQ ID NO: 21 - protein, 50 aa, Artificial Sequence
  BAP060- R022F
VVSHFNDCPDSHTQFCFHGTCFFLVQEDKPACVCHSGYVGARCEHADLLA

SEQ ID NO: 22 - protein, 50 aa, Artificial Sequence
  BAP060- R022G
VVSHFNDCPDSHTQFCFHGTCGFLVQEDKPACVCHSGYVGARCEHADLLA

SEQ ID NO: 23 - protein, 50 aa, Artificial Sequence
  BAP060- R022M
VVSHFNDCPDSHTQFCFHGTCMFLVQEDKPACVCHSGYVGARCEHADLLA

SEQ ID NO: 24 - protein, 50 aa, Artificial Sequence
  BAP060- R022N
VVSHFNDCPDSHTQFCFHGTCNFLVQEDKPACVCHSGYVGARCEHADLLA

SEQ ID NO: 25 - protein, 50 aa, Artificial Sequence
  BAP060- R022P
VVSHFNDCPDSHTQFCFHGTCPFLVQEDKPACVCHSGYVGARCEHADLLA

SEQ ID NO: 26 - protein, 50 aa, Artificial Sequence
  BAP060- R022Q
VVSHFNDCPDSHTQFCFHGTCQFLVQEDKPACVCHSGYVGARCEHADLLA

SEQ ID NO: 27 - protein, 50 aa, Artificial Sequence
  BAP060- R022T
VVSHFNDCPDSHTQFCFHGTCTFLVQEDKPACVCHSGYVGARCEHADLLA

SEQ ID NO: 28 - protein, 50 aa, Artificial Sequence
  BAP060- R022V
VVSHFNDCPDSHTQFCFHGTCVFLVQEDKPACVCHSGYVGARCEHADLLA

SEQ ID NO: 29 - protein, 50 aa, Artificial Sequence
  BAP060-F023A
VVSHFNDCPDSHTQFCFHGTCRALVQEDKPACVCHSGYVGARCEHADLLA

SEQ ID NO: 30 - protein, 50 aa, Artificial Sequence
  BAP060-F023P
VVSHFNDCPDSHTQFCFHGTCRPLVQEDKPACVCHSGYVGARCEHADLLA

SEQ ID NO: 31 - protein, 50 aa, Artificial Sequence
  BAP060-F023T
VVSHFNDCPDSHTQFCFHGTCRTLVQEDKPACVCHSGYVGARCEHADLLA

SEQ ID NO: 32 - protein, 50 aa, Artificial Sequence
  BAP060-L024E
VVSHFNDCPDSHTQFCFHGTCRFEVQEDKPACVCHSGYVGARCEHADLLA

SEQ ID NO: 33 - protein, 50 aa, Artificial Sequence
  BAP060-L024G
VVSHFNDCPDSHTQFCFHGTCRFGVQEDKPACVCHSGYVGARCEHADLLA

SEQ ID NO: 34 - protein, 50 aa, Artificial Sequence
  BAP060-L024S
VVSHFNDCPDSHTQFCFHGTCRFSVQEDKPACVCHSGYVGARCEHADLLA

SEQ ID NO: 35 - protein, 50 aa, Artificial Sequence
  BAP060-Q026D
VVSHFNDCPDSHTQFCFHGTCRFLVDEDKPACVCHSGYVGARCEHADLLA

SEQ ID NO: 36 - protein, 50 aa, Artificial Sequence
  BAP060-Q026E
VVSHFNDCPDSHTQFCFHGTCRFLVEEDKPACVCHSGYVGARCEHADLLA

SEQ ID NO: 37 - protein, 50 aa, Artificial Sequence
  BAP060-E027F
VVSHFNDCPDSHTQFCFHGTCRFLVQFDKPACVCHSGYVGARCEHADLLA

SEQ ID NO: 38 - protein, 50 aa, Artificial Sequence
  BAP060-E027I
VVSHFNDCPDSHTQFCFHGTCRFLVQIDKPACVCHSGYVGARCEHADLLA

SEQ ID NO: 39 - protein, 50 aa, Artificial Sequence
  BAP060-E027L
VVSHFNDCPDSHTQFCFHGTCRFLVQLDKPACVCHSGYVGARCEHADLLA

SEQ ID NO: 40 - protein, 50 aa, Artificial Sequence
  BAP060-E027Y
VVSHFNDCPDSHTQFCFHGTCRFLVQYDKPACVCHSGYVGARCEHADLLA

SEQ ID NO: 41 - protein, 50 aa, Artificial Sequence
  BAP060-D028F
VVSHFNDCPDSHTQFCFHGTCRFLVQEFKPACVCHSGYVGARCEHADLLA

SEQ ID NO: 42 - protein, 50 aa, Artificial Sequence
  BAP060-D028I
VVSHFNDCPDSHTQFCFHGTCRFLVQEIKPACVCHSGYVGARCEHADLLA

SEQ ID NO: 43 - protein, 50 aa, Artificial Sequence
  BAP060-D028K
VVSHFNDCPDSHTQFCFHGTCRFLVQEKKPACVCHSGYVGARCEHADLLA

SEQ ID NO: 44 - protein, 50 aa, Artificial Sequence
  BAP060-D028R
VVSHFNDCPDSHTQFCFHGTCRFLVQERKPACVCHSGYVGARCEHADLLA

SEQ ID NO: 45 - protein, 50 aa, Artificial Sequence
  BAP060-D028W
VVSHFNDCPDSHTQFCFHGTCRFLVQEWKPACVCHSGYVGARCEHADLLA

SEQ ID NO: 46 - protein, 50 aa, Artificial Sequence
  BAP060-K029E
VVSHFNDCPDSHTQFCFHGTCRFLVQEDEPACVCHSGYVGARCEHADLLA

SEQ ID NO: 47 - protein, 50 aa, Artificial Sequence
  BAP060-K029G
VVSHFNDCPDSHTQFCFHGTCRFLVQEDGPACVCHSGYVGARCEHADLLA

SEQ ID NO: 48 - protein, 50 aa, Artificial Sequence
  BAP060-K029L
VVSHFNDCPDSHTQFCFHGTCRFLVQEDLPACVCHSGYVGARCEHADLLA

SEQ ID NO: 49 - protein, 50 aa, Artificial Sequence
  BAP060-K029N
VVSHFNDCPDSHTQFCFHGTCRFLVQEDNPACVCHSGYVGARCEHADLLA

SEQ ID NO: 50 - protein, 50 aa, Artificial Sequence
  BAP060-K029P
VVSHFNDCPDSHTQFCFHGTCRFLVQEDPPACVCHSGYVGARCEHADLLA

SEQ ID NO: 51 - protein, 50 aa, Artificial Sequence
  BAP060-K029V
VVSHFNDCPDSHTQFCFHGTCRFLVQEDVPACVCHSGYVGARCEHADLLA

SEQ ID NO: 52 - protein, 50 aa, Artificial Sequence
  BAP060-K029W
VVSHFNDCPDSHTQFCFHGTCRFLVQEDWPACVCHSGYVGARCEHADLLA

SEQ ID NO: 53 - protein, 50 aa, Artificial Sequence
  BAP060-V033C
VVSHFNDCPDSHTQFCFHGTCRFLVQEDKPACCCHSGYVGARCEHADLLA

SEQ ID NO: 54 - protein, 50 aa, Artificial Sequence
  BAP060-V033E
VVSHFNDCPDSHTQFCFHGTCRFLVQEDKPACECHSGYVGARCEHADLLA

SEQ ID NO: 55 - protein, 50 aa, Artificial Sequence
  BAP060-V033S
VVSHFNDCPDSHTQFCFHGTCRFLVQEDKPACSCHSGYVGARCEHADLLA

SEQ ID NO: 56 - protein, 50 aa, Artificial Sequence
  BAP060-H035C
VVSHFNDCPDSHTQFCFHGTCRFLVQEDKPACVCCSGYVGARCEHADLLA

SEQ ID NO: 57 - protein, 50 aa, Artificial Sequence
  BAP060- H035D
VVSHFNDCPDSHTQFCFHGTCRFLVQEDKPACVCDSGYVGARCEHADLLA

SEQ ID NO: 58 - protein, 50 aa, Artificial Sequence
  BAP060- H035E
VVSHFNDCPDSHTQFCFHGTCRFLVQEDKPACVCESGYVGARCEHADLLA

SEQ ID NO: 59 - protein, 50 aa, Artificial Sequence
  BAP060- H035G
VVSHFNDCPDSHTQFCFHGTCRFLVQEDKPACVCGSGYVGARCEHADLLA

SEQ ID NO: 60 - protein, 50 aa, Artificial Sequence
  BAP060- H035Q
VVSHFNDCPDSHTQFCFHGTCRFLVQEDKPACVCQSGYVGARCEHADLLA

SEQ ID NO: 61 - protein, 50 aa, Artificial Sequence
  BAP060-S036K
VVSHFNDCPDSHTQFCFHGTCRFLVQEDKPACVCHKGYVGARCEHADLLA

SEQ ID NO: 62 - protein, 50 aa, Artificial Sequence
  BAP060- S036N
VVSHFNDCPDSHTQFCFHGTCRFLVQEDKPACVCHNGYVGARCEHADLLA

SEQ ID NO: 63 - protein, 50 aa, Artificial Sequence
  BAP060- S036R
VVSHFNDCPDSHTQFCFHGTCRFLVQEDKPACVCHRGYVGARCEHADLLA

SEQ ID NO: 64 - protein, 50 aa, Artificial Sequence
  BAP060-Y038D
VVSHFNDCPDSHTQFCFHGTCRFLVQEDKPACVCHSGDVGARCEHADLLA

SEQ ID NO: 65 - protein, 50 aa, Artificial Sequence
  BAP060- Y038E
VVSHFNDCPDSHTQFCFHGTCRFLVQEDKPACVCHSGEVGARCEHADLLA

SEQ ID NO: 66 - protein, 50 aa, Artificial Sequence
  BAP060- Y038I
VVSHFNDCPDSHTQFCFHGTCRFLVQEDKPACVCHSGIVGARCEHADLLA

SEQ ID NO: 67 - protein, 50 aa, Artificial Sequence
  BAP060- Y038M
VVSHFNDCPDSHTQFCFHGTCRFLVQEDKPACVCHSGMVGARCEHADLLA

SEQ ID NO: 68 - protein, 50 aa, Artificial Sequence
  BAP060- Y038T
VVSHFNDCPDSHTQFCFHGTCRFLVQEDKPACVCHSGTVGARCEHADLLA

SEQ ID NO: 69 - protein, 50 aa, Artificial Sequence
  BAP060-G040D
VVSHFNDCPDSHTQFCFHGTCRFLVQEDKPACVCHSGYVDARCEHADLLA

SEQ ID NO: 70 - protein, 50 aa, Artificial Sequence
  BAP060-G040E
VVSHFNDCPDSHTQFCFHGTCRFLVQEDKPACVCHSGYVEARCEHADLLA

SEQ ID NO: 71 - protein, 50 aa, Artificial Sequence
  BAP060-G040K
VVSHFNDCPDSHTQFCFHGTCRFLVQEDKPACVCHSGYVKARCEHADLLA

SEQ ID NO: 72 - protein, 50 aa, Artificial Sequence
  BAP060-G040Q
VVSHFNDCPDSHTQFCFHGTCRFLVQEDKPACVCHSGYVQARCEHADLLA

SEQ ID NO: 73 - protein, 50 aa, Artificial Sequence
  BAP060-G040S
VVSHFNDCPDSHTQFCFHGTCRFLVQEDKPACVCHSGYVSARCEHADLLA

SEQ ID NO: 74 - protein, 50 aa, Artificial Sequence
  BAP060-A041D
VVSHFNDCPDSHTQFCFHGTCRFLVQEDKPACVCHSGYVGDRCEHADLLA

SEQ ID NO: 75 - protein, 50 aa, Artificial Sequence
  BAP060-A041F
VVSHFNDCPDSHTQFCFHGTCRFLVQEDKPACVCHSGYVGFRCEHADLLA

SEQ ID NO: 76 - protein, 50 aa, Artificial Sequence
  BAP060-A041H
VVSHFNDCPDSHTQFCFHGTCRFLVQEDKPACVCHSGYVGHRCEHADLLA

SEQ ID NO: 77 - protein, 50 aa, Artificial Sequence
  BAP060-A041S
VVSHFNDCPDSHTQFCFHGTCRFLVQEDKPACVCHSGYVGSRCEHADLLA

SEQ ID NO: 78 - protein, 50 aa, Artificial Sequence
  BAP060-A041W
VVSHFNDCPDSHTQFCFHGTCRFLVQEDKPACVCHSGYVGWRCEHADLLA

SEQ ID NO: 79 - protein, 50 aa, Artificial Sequence
  BAP060-A041Y
VVSHFNDCPDSHTQFCFHGTCRFLVQEDKPACVCHSGYVGYRCEHADLLA

SEQ ID NO: 80 - protein, 50 aa, Artificial Sequence
  BAP060-E044M
VVSHFNDCPDSHTQFCFHGTCRFLVQEDKPACVCHSGYVGARCMHADLLA

SEQ ID NO: 81 - protein, 50 aa, Artificial Sequence
  BAP060-E044Q
VVSHFNDCPDSHTQFCFHGTCRFLVQEDKPACVCHSGYVGARCQHADLLA

SEQ ID NO: 82 - protein, 50 aa, Artificial Sequence
  BAP060-E044S
VVSHFNDCPDSHTQFCFHGTCRFLVQEDKPACVCHSGYVGARCSHADLLA

SEQ ID NO: 83 - protein, 50 aa, Artificial Sequence
  BAP060-A046I
VVSHFNDCPDSHTQFCFHGTCRFLVQEDKPACVCHSGYVGARCEHIDLLA

SEQ ID NO: 84 - protein, 50 aa, Artificial Sequence
  BAP060-A046L
VVSHFNDCPDSHTQFCFHGTCRFLVQEDKPACVCHSGYVGARCEHLDLLA

SEQ ID NO: 85 - protein, 50 aa, Artificial Sequence
  BAP060-A046V
VVSHFNDCPDSHTQFCFHGTCRFLVQEDKPACVCHSGYVGARCEHVDLLA

SEQ ID NO: 86 - protein, 50 aa, Artificial Sequence
  BAP060-A046Y
VVSHFNDCPDSHTQFCFHGTCRFLVQEDKPACVCHSGYVGARCEHYDLLA

SEQ ID NO: 87 - protein, 50 aa, Artificial Sequence
  BAP060-D047N
VVSHFNDCPDSHTQFCFHGTCRFLVQEDKPACVCHSGYVGARCEHANLLA

SEQ ID NO: 88 - protein, 50 aa, Artificial Sequence
  BAP060-D047W
VVSHFNDCPDSHTQFCFHGTCRFLVQEDKPACVCHSGYVGARCEHAWLLA

SEQ ID NO: 89 - protein, 50 aa, Artificial Sequence
  BAP060-L048D
VVSHFNDCPDSHTQFCFHGTCRFLVQEDKPACVCHSGYVGARCEHADDLA

SEQ ID NO: 90 - protein, 50 aa, Artificial Sequence
  BAP060-L048K
VVSHFNDCPDSHTQFCFHGTCRFLVQEDKPACVCHSGYVGARCEHADKLA

SEQ ID NO: 91 - protein, 50 aa, Artificial Sequence
  BAP060-A050F
VVSHFNDCPDSHTQFCFHGTCRFLVQEDKPACVCHSGYVGARCEHADLLF

SEQ ID NO: 92 - protein, 50 aa, Artificial Sequence
  BAP060-A050H
VVSHFNDCPDSHTQFCFHGTCRFLVQEDKPACVCHSGYVGARCEHADLLH

SEQ ID NO: 93 - protein, 50 aa, Artificial Sequence
  BAP060-A050K
VVSHFNDCPDSHTQFCFHGTCRFLVQEDKPACVCHSGYVGARCEHADLLK

SEQ ID NO: 94 - protein, 50 aa, Artificial Sequence
  BAP060-A050R
VVSHFNDCPDSHTQFCFHGTCRFLVQEDKPACVCHSGYVGARCEHADLLR

SEQ ID NO: 95 - protein, 50 aa, Artificial Sequence
  BAP060-A050W
VVSHFNDCPDSHTQFCFHGTCRFLVQEDKPACVCHSGYVGARCEHADLLW

SEQ ID NO: 96 - protein, 50 aa, Artificial Sequence
  Q14G-F17L
VVSHFNDCPDSHTGFCLHGTCRFLVQEDKPACVCHSGYVGARCEHADLLA

SEQ ID NO: 97 - protein, 50 aa, Artificial Sequence
  Q14G-A41D
VVSHFNDCPDSHTGFCFHGTCRFLVQEDKPACVCHSGYVGDRCEHADLLA

SEQ ID NO: 98 - protein, 50 aa, Artificial Sequence
  S36R-A46Y
VVSHFNDCPDSHTQFCFHGTCRFLVQEDKPACVCHRGYVGARCEHYDLLA

SEQ ID NO: 99 - protein, 50 aa, Artificial Sequence
  E27L-A46Y
VVSHFNDCPDSHTQFCFHGTCRFLVQLDKPACVCHSGYVGARCEHYDLLA

SEQ ID NO: 100 - protein, 50 aa, Artificial Sequence
  F17L-E27L
VVSHFNDCPDSHTQFCLHGTCRFLVQLDKPACVCHSGYVGARCEHADLLA

SEQ ID NO: 101 - protein, 50 aa, Artificial Sequence
  A46Y-A50W
VVSHFNDCPDSHTQFCFHGTCRFLVQEDKPACVCHSGYVGARCEHYDLLW

SEQ ID NO: 102 - protein, 50 aa, Artificial Sequence
  F17L-A50W
VVSHFNDCPDSHTQFCLHGTCRFLVQEDKPACVCHSGYVGARCEHADLLW

SEQ ID NO: 103 - protein, 50 aa, Artificial Sequence
  E27L-A50W
VVSHFNDCPDSHTQFCFHGTCRFLVQLDKPACVCHSGYVGARCEHADLLW

SEQ ID NO: 104 - protein, 50 aa, Artificial Sequence
  Q14G-A46Y-A50W
VVSHFNDCPDSHTGFCFHGTCRFLVQEDKPACVCHSGYVGARCEHYDLLW

SEQ ID NO: 105 - protein, 50 aa, Artificial Sequence
  Q14G-E27L-S36R
VVSHFNDCPDSHTGFCFHGTCRFLVQLDKPACVCHRGYVGARCEHADLLA

SEQ ID NO: 106 - protein, 50 aa, Artificial Sequence
  E27L-A41D-A46Y
VVSHFNDCPDSHTQFCFHGTCRFLVQLDKPACVCHSGYVGDRCEHYDLLA

SEQ ID NO: 107 - protein, 50 aa, Artificial Sequence
  F17L-S36R-A50W
VVSHFNDCPDSHTQFCLHGTCRFLVQEDKPACVCHRGYVGARCEHADLLW

SEQ ID NO: 108 - protein, 50 aa, Artificial Sequence
  F17L-E27L-A50W
VVSHFNDCPDSHTQFCLHGTCRFLVQLDKPACVCHSGYVGARCEHADLLW

SEQ ID NO: 109 - protein, 50 aa, Artificial Sequence
  E27L-S36R-A46Y
VVSHFNDCPDSHTQFCFHGTCRFLVQLDKPACVCHRGYVGARCEHYDLLA

SEQ ID NO: 110 - protein, 50 aa, Artificial Sequence
  F17L-S36R-A41D
VVSHFNDCPDSHTQFCLHGTCRFLVQEDKPACVCHRGYVGDRCEHADLLA

SEQ ID NO: 111 - protein, 50 aa, Artificial Sequence
  Q14G-E27L-A46Y
VVSHFNDCPDSHTGFCFHGTCRFLVQLDKPACVCHSGYVGARCEHYDLLA

SEQ ID NO: 112 - protein, 50 aa, Artificial Sequence
  F17L-S36R-A41D-A46Y
VVSHFNDCPDSHTQFCLHGTCRFLVQEDKPACVCHRGYVGDRCEHYDLLA

SEQ ID NO: 113 - protein, 50 aa, Artificial Sequence
  Q14G-F17L-S36R-A46Y
VVSHFNDCPDSHTGFCLHGTCRFLVQEDKPACVCHRGYVGARCEHYDLLA

SEQ ID NO: 114 - protein, 50 aa, Artificial Sequence
  F17L-E27L-A41D-A46Y
VVSHFNDCPDSHTQFCLHGTCRFLVQLDKPACVCHSGYVGDRCEHYDLLA

SEQ ID NO: 115 - protein, 50 aa, Artificial Sequence
  F17L-S36R-A46Y-A50W
VVSHFNDCPDSHTQFCLHGTCRFLVQEDKPACVCHRGYVGARCEHYDLLW

SEQ ID NO: 116 - protein, 50 aa, Artificial Sequence
  E27L-S36R-A46Y-A50W
VVSHFNDCPDSHTQFCFHGTCRFLVQLDKPACVCHRGYVGARCEHYDLLW

SEQ ID NO: 117 - protein, 50 aa, Artificial Sequence
  E27L-S36R-A41D-A50W
VVSHFNDCPDSHTQFCFHGTCRFLVQLDKPACVCHRGYVGDRCEHADLLW

SEQ ID NO: 118 - protein, 50 aa, Artificial Sequence
  F17L-E27L-S36R-A46Y
VVSHFNDCPDSHTQFCLHGTCRFLVQLDKPACVCHRGYVGARCEHYDLLA

SEQ ID NO: 119 - protein, 50 aa, Artificial Sequence
  Q14G-E27L-S36R-A41D
VVSHFNDCPDSHTGFCFHGTCRFLVQLDKPACVCHRGYVGDRCEHADLLA

SEQ ID NO: 120 - protein, 50 aa, Artificial Sequence
  Q14G-A41D-A46Y-A50W
VVSHFNDCPDSHTGFCFHGTCRFLVQEDKPACVCHSGYVGDRCEHYDLLW

SEQ ID NO: 121 - protein, 50 aa, Artificial Sequence
  Q14G-E27L-A41D-A46Y
VVSHFNDCPDSHTGFCFHGTCRFLVQLDKPACVCHSGYVGDRCEHYDLLA

SEQ ID NO: 122 - protein, 50 aa, Artificial Sequence
  Q14G-S36R-A46Y-A50W
VVSHFNDCPDSHTGFCFHGTCRFLVQEDKPACVCHRGYVGARCEHYDLLW

SEQ ID NO: 123 - protein, 50 aa, Artificial Sequence
  Q14G-F17L-E27L-A46Y
VVSHFNDCPDSHTGFCLHGTCRFLVQLDKPACVCHSGYVGARCEHYDLLA

SEQ ID NO: 124 - protein, 50 aa, Artificial Sequence
  Q14G-F17L-A46Y-A50W
VVSHFNDCPDSHTGFCLHGTCRFLVQEDKPACVCHSGYVGARCEHYDLLW

SEQ ID NO: 125 - protein, 50 aa, Artificial Sequence
  Q14G-F17L-E27L-A41D
VVSHFNDCPDSHTGFCLHGTCRFLVQLDKPACVCHSGYVGDRCEHADLLA

SEQ ID NO: 126 - protein, 50 aa, Artificial Sequence
  F17L-E27L-A46Y-A50W
VVSHFNDCPDSHTQFCLHGTCRFLVQLDKPACVCHSGYVGARCEHYDLLW

SEQ ID NO: 127 - protein, 50 aa, Artificial Sequence
  Q14G-F17L-E27L-A50W
VVSHFNDCPDSHTGFCLHGTCRFLVQLDKPACVCHSGYVGARCEHADLLW

SEQ ID NO: 128 - protein, 50 aa, Artificial Sequence
  Q14G-F17L-E27L-S36R-A46Y
VVSHFNDCPDSHTGFCLHGTCRFLVQLDKPACVCHRGYVGARCEHYDLLA

SEQ ID NO: 129 - protein, 50 aa, Artificial Sequence
  Q14G-F17L-A41D-A46Y-A50W
VVSHFNDCPDSHTGFCLHGTCRFLVQEDKPACVCHSGYVGDRCEHYDLLW

SEQ ID NO: 130 - protein, 50 aa, Artificial Sequence
  Q14G-S36R-A41D-A46Y-A50W
VVSHFNDCPDSHTGFCFHGTCRFLVQEDKPACVCHRGYVGDRCEHYDLLW

SEQ ID NO: 131 - protein, 50 aa, Artificial Sequence
  Q14G-E27L-S36R-A41D-A50W
VVSHFNDCPDSHTGFCFHGTCRFLVQLDKPACVCHRGYVGDRCEHADLLW

SEQ ID NO: 132 - protein, 50 aa, Artificial Sequence
  F17L-E27L-S36R-A41D-A46Y
VVSHFNDCPDSHTQFCLHGTCRFLVQLDKPACVCHRGYVGDRCEHYDLLA

SEQ ID NO: 133 - protein, 50 aa, Artificial Sequence
  F17L-S36R-A41D-A46Y-A50W
VVSHFNDCPDSHTQFCLHGTCRFLVQEDKPACVCHRGYVGDRCEHYDLLW

SEQ ID NO: 134 - protein, 50 aa, Artificial Sequence
  Q14G-F17L-S36R-A41D-A50W
VVSHFNDCPDSHTGFCLHGTCRFLVQEDKPACVCHRGYVGDRCEHADLLW

SEQ ID NO: 135 - protein, 50 aa, Artificial Sequence
  Q14G-F17L-E27L-A41D-A50W
VVSHFNDCPDSHTGFCLHGTCRFLVQLDKPACVCHSGYVGDRCEHADLLW

SEQ ID NO: 136 - protein, 50 aa, Artificial Sequence
  E27L-S36R-A41D-A46Y-A50W
VVSHFNDCPDSHTQFCFHGTCRFLVQLDKPACVCHRGYVGDRCEHYDLLW

SEQ ID NO: 137 - protein, 50 aa, Artificial Sequence
  Q14G-F17L-E27L-A46Y-A50W
VVSHFNDCPDSHTGFCLHGTCRFLVQLDKPACVCHSGYVGARCEHYDLLW

SEQ ID NO: 138 - protein, 50 aa, Artificial Sequence
  Q14G-F17L-E27L-A41D-A46Y
VVSHFNDCPDSHTGFCLHGTCRFLVQLDKPACVCHSGYVGDRCEHYDLLA

SEQ ID NO: 139 - protein, 50 aa, Artificial Sequence
  F17L-E27L-S36R-A41D-A50W
VVSHFNDCPDSHTQFCLHGTCRFLVQLDKPACVCHRGYVGDRCEHADLLW

SEQ ID NO: 140 - protein, 50 aa, Artificial Sequence
  F17L-E27L-S36R-A41D-A46Y-A50W
VVSHFNDCPDSHTQFCLHGTCRFLVQLDKPACVCHRGYVGDRCEHYDLLW

SEQ ID NO: 141 - protein, 50 aa, Artificial Sequence
  Q14G-F17L-E27L-A41D-A46Y-A50W
VVSHFNDCPDSHTGFCLHGTCRFLVQLDKPACVCHSGYVGDRCEHYDLLW

SEQ ID NO: 142 - protein, 50 aa, Artificial Sequence
  Q14G-F17L-E27L-S36R-A41D-A50W
VVSHFNDCPDSHTGFCLHGTCRFLVQLDKPACVCHRGYVGDRCEHADLLW

SEQ ID NO: 143 - protein, 50 aa, Artificial Sequence
  Q14G-F17L-E27L-S36R-A41D-A46Y-A50W
VVSHFNDCPDSHTGFCLHGTCRFLVQLDKPACVCHRGYVGDRCEHYDLLW

SEQ ID NO: 144 - DNA, 216 bp, Homo sapiens
atgaaatacctgctgccgaccgctgctgctggtctgctgctcctcgctgcccagccggcgatggccgtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggcc

SEQ ID NO: 145 - protein, 72 aa, Homo sapiens
MKYLLPTAAAGLLLLAAQPAMAVVSHFNDCPDSHTQFCFHGTCRFLVQEDKPACVCHSGYVGARCEHADLLA

SEQ ID NO: 146 - DNA, 218 bp, Homo sapiens
atggacatgagggtccccgctcagctcctggggctcctgctgctctggctcccaggtgccaagtgtgtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgc

SEQ ID NO: 147 - protein, 72 aa, Homo sapiens
MDMRVPAQLLGLLLLWLPGAKCVVSHFNDCPDSHTQFCFHGTCRFLVQEDKPACVCHSGYVGARCEHADLLA

SEQ ID NO: 148 - DNA, 482 bp, Homo sapiens
atggacatgagggtccccgctcagctcctggggctcctgctgctctggctcccaggtgccaagtgtttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtcgc

SEQ ID NO: 149 - protein, 160 aa, Homo sapiens
MDMRVPAQLLGLLLLWLPGAKCLENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV